(19) United States
(12) Patent Application Publication
(10) Pub. No.: US 2026/0028342 A1
(43) Pub. Date: Jan. 29, 2026

(54) ENHANCING ANTIMETASTASIS ACTIVITY USING TARGETED PROTEIN DEGRADATION

(71) Applicants: THE UNIVERSITY OF NORTH CAROLINA AT CHAPEL HILL, Chapel Hill, NC (US); Northwestern University, Evanston, IL (US)

(72) Inventors: Kevin Frankowski, Pittsboro, NC (US); Feijun Wang, Cary, NC (US); Sui Huang, Chicago, IL (US); Emma Freeman, Chicago, IL (US)

(21) Appl. No.: 18/997,325

(22) PCT Filed: Jul. 28, 2023
(86) PCT No.: PCT/US23/28929

§ 371 Date: Jan. 21, 2025

Related U.S. Application Data
(60) Provisional application No. 63/393,449, filed on Jul. 29, 2022.

(51) Int. Cl. C07D 487/04; A61K 31/519; A61K 47/55; A61P 35/00

(52) U.S. Cl. CPC C07D 487/04; A61K 31/519; A61K 47/55; A61P 35/00

(57) ABSTRACT

The current invention relates to transcription elongation factor eEF1A2-targeted protein degradation ligands and pharmaceutical compositions thereof and their utility as anti-cancer agents.

### FIELD OF THE INVENTION

The present invention is directed to translation elongation factor eEF1A2-targeted protein degradation ligands and pharmaceutical compositions thereof and their utility as anti-cancer agents.

### BACKGROUND

Although advances in cancer detection and treatment have reduced overall mortality, cancer remains the second most deadly disease in the U.S., due mainly to metastasis [1-3]. A subset of cancers, including pancreatic and metastatic breast cancer, are disproportionately lethal, primarily because current cancer management is often ineffective against metastasis. In 2016 alone, 246,000 women in the U.S. developed invasive breast cancer [12]. The five-year survival rate for metastatic breast cancer patients is only 27%, in contrast to 99% for stage I breast cancer patients [13]. Among pancreatic cancer patients, 90% die within one year of diagnosis [14-16].

Although cancer cells are highly heterogeneous throughout their evolution, metastatic cells do share a common lethal feature: their metastatic capability. This unique property distinguishes metastatic cells from non-metastatic tumor or normal cells at any stage of development. Given the limited understanding of metastatic mechanisms and limited results from target-driven drug development, alternative approaches should be considered. Surrogate markers that represent the complex but unique characteristics of metastatic cancer cells could reflect the metastatic phenotype better than a single gene or gene product. Such surrogate markers could direct the search for effective compounds, which, in turn, could provide tools to identify key mechanisms of metastasis.

For example, one such marker identifying cancer cells competent to metastasize is the perinucleolar compartment (PNC). The PNC is a multicomponent, irregularly shaped subnuclear structure (FIG. 1). It is associated with, but structurally distinct from, the nucleolus. The PNC is present predominantly in cancer cells and absent from normal cells, including embryonic mouse and human stem cells. Although the complete molecular composition of the PNC and its function remains unknown, it contains small RNAs transcribed by RNA polymerase III(Pol III), and RNA-binding proteins primarily implicated in Pol II RNA processing [20-23]. The PNC is heavily involved in RNA synthesis and metabolism as BrU pulse labeling experiments have shown that it enriches with newly synthesized RNA [24] and associates with chromatin [25]. While formation of PNCs is not related to the rate of proliferation, or the state of differentiation, PNC prevalence (percentage of cells containing at least 1 PNC) correlates with the metastatic capability of cancer cells both in vitro and in vivo [17, 19, 26]. PNC prevalence positively correlates with disease progression and negatively correlates with patient prognosis in several cancers tested, including breast [17], ovarian, and colon cancers [26]. Thus, PNC prevalence is a pan-tumor biomarker that reflects the malignant behaviors of cancer cells arising from solid tissues.

Since very few cancer treatments are effective for metastatic diseases, there is an urgent need to not only develop new therapeutic approaches to treat metastasis, but also to employ new biomarkers, such as PNC and other cellular targets, for the identification and treatment of metastatic diseases.

### SUMMARY

Provided herein are ligands which can bind to the translation elongation factor eEF1A2 protein in the non-active site and degrade the protein, or which can bind to the protein in the active site and inhibit the function of the protein. Thus, one aspect of the current disclosure is directed to a compound of Formula (I):

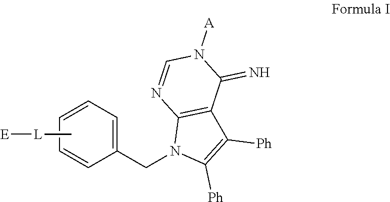

or an enantiomer, diastereomer, an enantiomeric mixture, a diastereomeric mixture or a pharmaceutically acceptable salt thereof; wherein:
A is selected from

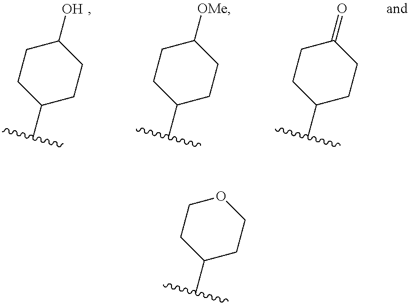 [SMILES: *C1CCC(O)CC1 ; *C1CCC(OC)CC1 ; *C1CCC(=O)CC1 ; *C1CCOCC1]

L is selected from

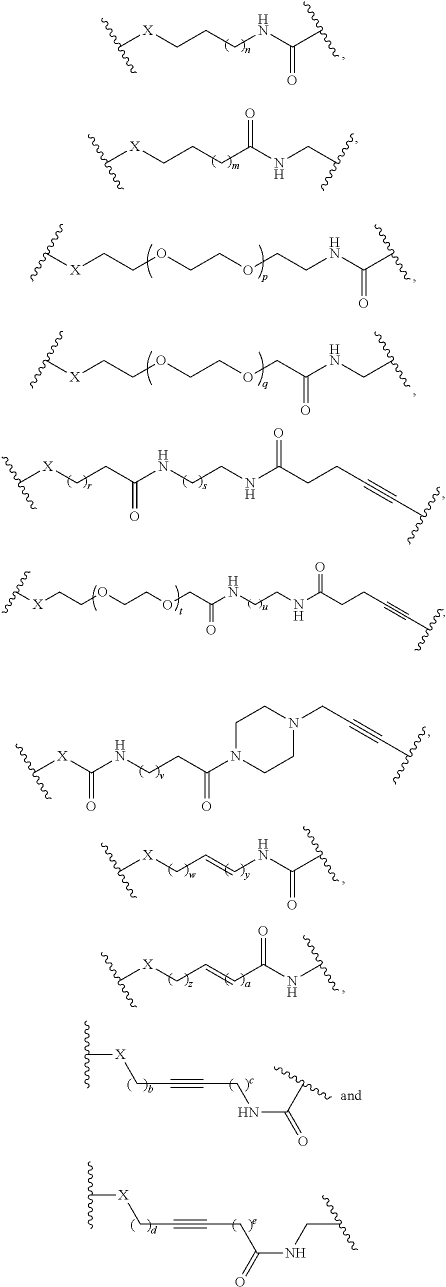

wherein a, b, c, d, e, n, m, p, q, r, s, t, u, v, w, y and z are an integer independently selected from 0, 1, 2, 3, 4, 5, 6, 7, 8, 9, and 10, and X is selected from —CH₂—, —NH—, —O— and a bond; and
E is selected from

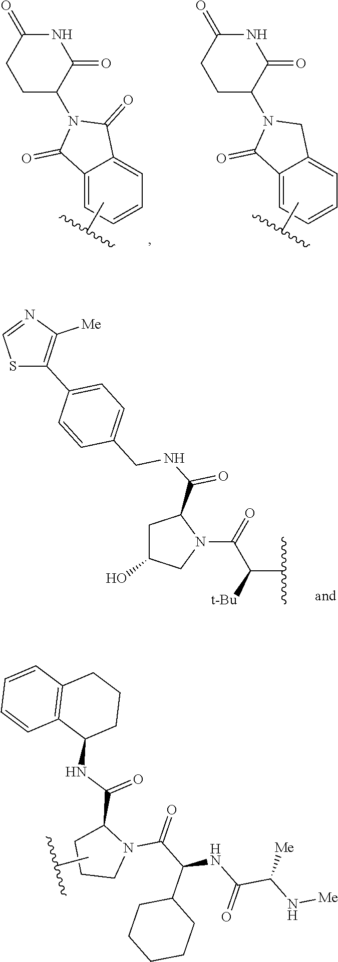

Another aspect of the disclosure is directed to a compound of formula (IV): A compound of formula (IV):

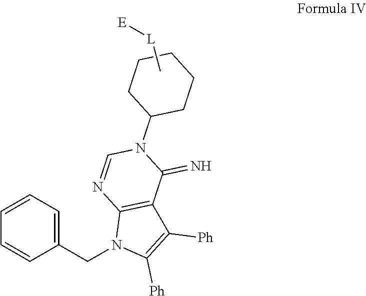

or an enantiomer, diastereomer, an enantiomeric mixture, a diastereomeric mixture or a pharmaceutically acceptable salt thereof; wherein:
L is selected from

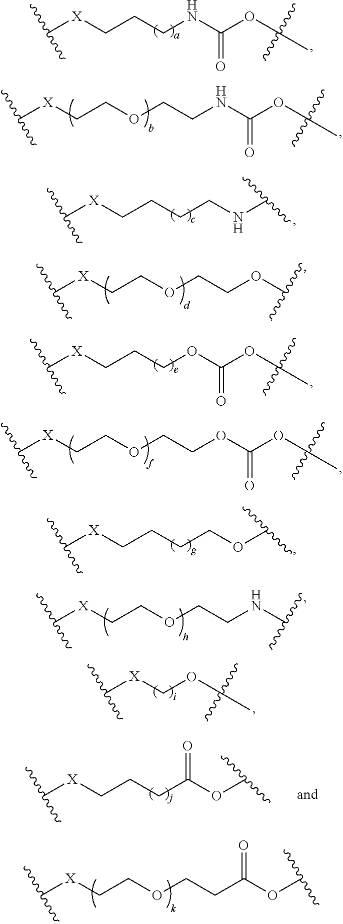

wherein g, d, i, j, and k are an integer independently selected from 0, 1, 2, 3, 4, 5, 6, 7, and 8, and X is selected from a bond —CH₂—, —NH—, and —O—, and
E is selected from

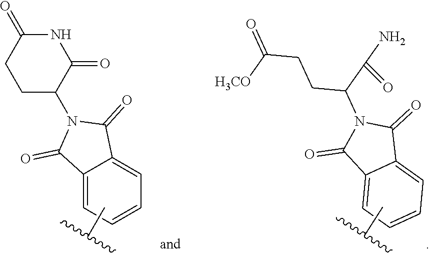

In some embodiments, X is selected from —NH— and —O—.

Another aspect of the disclosure is directed to a pharmaceutical composition comprising a compound as disclosed herein or a pharmaceutically acceptable salt thereof, and one or more pharmaceutically acceptable carrier(s).

Another aspect of the disclosure is directed to a method for treating a disease or condition that is treatable by inhibition of the translation elongation factor eEF1A2 protein, the method comprising administering to a subject in need thereof of a therapeutically effective amount of a compound or a pharmaceutical composition as disclosed herein. In some embodiments, the disease is cancer, such as metastatic cancer.

### DESCRIPTION OF THE DRAWINGS

FIG. 1 shows PNC images. The left panel shows microscope images of GFP-PTB marking the PNC in light grey, nucleoli in grey, and nuclear envelope in dark grey. The right panel is an electron microscopy image of a PNC-associated nucleolus with distinct morphology in optimally fixed cells. Arrows indicate the PNC.

FIGS. 2A-2F show metarrestin and various studies. FIG. 2A shows the chemical structure of metarrestin; FIG. 2B shows that metarrestin at doses of 5 mg/kg and 25 mg/kg inhibits metastasis in a PANC1 metastatic model as a function of organ/tumor ratio in liver and lung; FIG. 2C shows that metarrestin at doses of 25 mg/kg inhibits metastasis in a PANC1 metastatic model measured as a function of metastatic deposits in liver and lung; FIG. 2D compares liver tissue exposed to vehicle compared to 25 mg/kg of metarrestin; FIG. 2E shows results of a study where metarrestin treatment began prior to macrometastasis, which afforded drastic animal survival advantages; and FIG. 2F shows results of a study where metarrestin treatment began after macrometastasis establishment remained effective at extending survival of tumor-bearing animals with reduced metastatic organ replacement. **p<0.01, *p<0.05 (20); and FIG. 2G shows images of liver, lung and pancreas of animals treated with metarrestin from study in FIG. 2F compared to vehicle illustrating that less tumors were present in such organs in treated animals.

FIGS. 3A-3E show metarrestin binding and cellular data. FIG. 3A presents data showing that metarrestin specifically binds eEF1A2, and increased eEF1A2 enhances PNCs and metastasis formation. Here, metarrestin effectively outcompeted recombinant eEF1A from binding to an anchored biotinylated metarrestin-eEF1A complex; FIG. 3B presents data showing metarrestin treatment that stabilized eEF1A in a thermal stability assay using PC3M cell lysate; FIG. 3C presents data showing overexpression of HA-eEF1A2 enhanced PNC structures in cells (image panels, each containing a single nucleus). While it did not significantly increase overall PNC prevalence (top), overexpression of HA-eEF1A2 increased the number of PNCs per nucleus (scattered PNC prevalence: the number of cells containing two or more PNCs); n=300 cells (bar=2 μm); FIG. 3D presents data showing eEF1A2 overexpression in PC3M cells increased the IC₅₀ of metarrestin for PNC disassembly; and FIG. 3E presents data showing a study where 6×10⁴ PANC1 3D spheres transduced with empty vector (control) or eEF1A2 (eEF1A2 O.E.) were injected into the tail of the pancreas of NSG mice. Mice from both groups were harvested six weeks after implantation and subjected to necropsy. Macroscopic images of anterior (top) and posterior (bottom) liver surfaces showed higher metastatic burden in PANC eEF1A2 animals than in empty vector control (left, harvested livers). Histopathological images (H&E) of livers (black scale bar=250 μm, white scale bar=100 μm) are shown on the right. Insets depict representative metastatic lesions. Quantification of liver metastasis showed a higher metastatic burden in PANC1 eEF1A2 O.E. animals (n=4 animals analyzed).

FIG. 4 shows an exemplary general chemical structure of an eEF1A2 ligand disclosed herein containing a metarrestin binding moiety, a linker, and an E3 ligase binding moiety.

FIG. 5 shows the process of PROTAC-mediated ubiquitination and proteasomal degradation of the protein of interest (POI). PROTAC is composed of a ligand that binds to the E3 ubiquitin ligase and a ligand that binds to the target protein through a linker, which can induce the polyubiquitination and proteasome degradation of the target proteins in cells.

FIGS. 6A and 6B show results from metarrestin-PROTAC studies. FIG. 6A shows results of a study wherein metarrestin-PROTAC derivatives demonstrate improved potency for PNC inhibition, i.e., efficacy against PNCs; and FIG. 6B shows results of a study wherein metarrestin-PROTAC derivatives demonstrate improved potency for PNC inhibition, i.e., Compound 49 treatment for 20 hours reduced eEF1A2 by Western blot analysis. The density quantification uses actin as the loading control and vehicle treatment as standard of one, which shows a significant reduction of the protein eEF1A2.

FIG. 7 shows the results of as assay where HeLA cells were treated with compounds 56 or 57 (at 0.1 or 0.5 M concentrations) and the percentage of cells with PNC's were measured. PNC prevalence is correlated with metastatic potential and can be disrupted through modulation of the elongation factor eEF1A. Thus it is notable that at 0.1 μM both 56 and 57 show measureable reduction in PNC prevalence and at 0.5 M both 56 and 57 effect almost complete disruption of PNC prevalence.

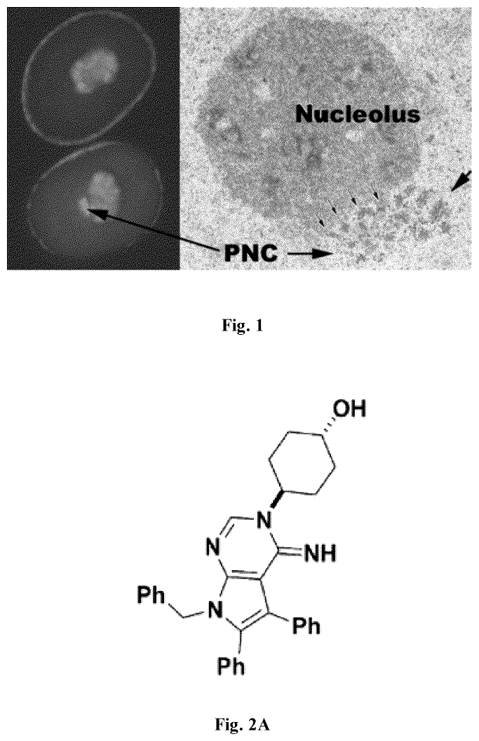

### DETAILED DESCRIPTION

The presently disclosed subject matter will now be described more completely hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

Metastasis is the cellular mechanism used by disease to spread from an organ to another non-adjacent part of the organism. This process is particularly important in the development of solid tumors and is responsible for the majority of deaths associated with this disease. It is well recognized in the field that treatment of a tumoral lesion has a better prognosis if started in a pre-metastatic stage.

Metarrestin, a small molecule PNC inhibitor, has shown potent in vitro anti-oncogenic properties, including migration and invasion inhibition, and anti-metastatic activity. However, although metarrestin exhibits these desirable properties, at overloading dosing reversible seizures in large mammal dose-escalation studies were observed. Thus, the potential for dose-limiting neurological side effects could constrain the patient population to drug-resistant or late-stage individuals. However, cancer patient outcomes would generally greatly benefit from adjuvant or co-administration use of an anti-metastasis agent such as metarrestin at any stage of cancer. Therefore, in order to improve the cancer cell selectivity and reduce potential toxicity to normal cells, development of next generation metarrestin derivatives that selectively enhance drug efficacy against cancer cells over normal cells were needed.

As reported herein is a strategy to exploit protein expression alterations of cancer cells using proteolysis targeting chimeras (PROTACs). Compared with traditional pharmaceutical drug discovery focused on directly controlling protein activity, PROTAC utilizes proteasome-mediated proteolysis via E3 ligase and engages the body's housekeeping protein degradation system to selectively degrade the pathogenic proteins, thereby treating cancer or other difficult-to-treat diseases. PROTAC is a heterobifunctional small molecule that consists of a linker and two warheads—one binds to the target protein, and the other recruits the E3 ligase (see FIG. 5). The PROTAC molecules combine the target protein with E3 ligase together to form a ternary complex, which induces E3 ligase ubiquitination of the target protein to initiate the degradation process. The ubiquitinated target protein is recognized and degraded by 26S proteasome, which is a part of the eukaryotic cells of the ubiquitin/proteasome system. The ability of PROTACs to induce the degradation of the target protein is not limited to the binding site within the kinase domain, and it may be achieved when the kinase activity is not the singular action of the target protein. Empowered by this bifunctional characteristic, the disease-causing proteins can be ubiquitinated and degraded by the proteasome, which will result in a return to normal physiological tissue.

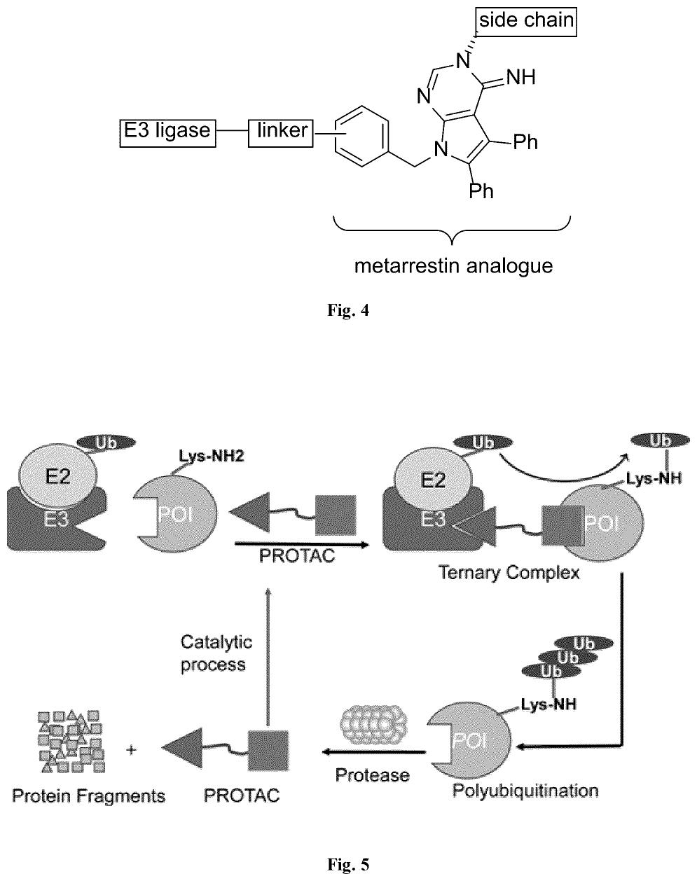

The compounds disclosed herein were constructed using this PROTAC methodology to afford PROTAC derivatives [30-33], which degrade metarrestin targets using eEF1A2, a validated target for the anti-metastasis mechanism of metarrestin. Specifically, the compounds disclosed herein contain a metarrestin or an analogue thereof conjugated via a linker to an E3 ligase ligand (see FIG. 4). Advantages for employing such a PROTAC conjugation with metarrestin analogues include, but are not limited to: 1) it will provide more potent metarrestin derivatives that would lower the requisite dosing levels; and 2) the tethering of an E3 ligase ligand to metarrestin is likely to reduce the CNS penetration of the PROTAC derivative and, thus, limit potential neurological side effects.

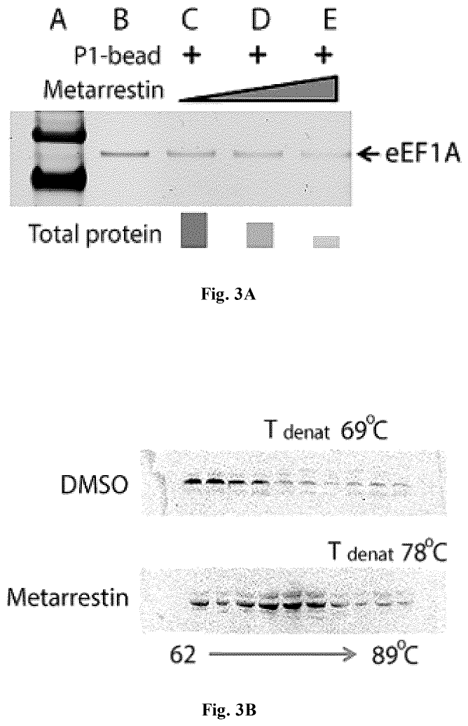

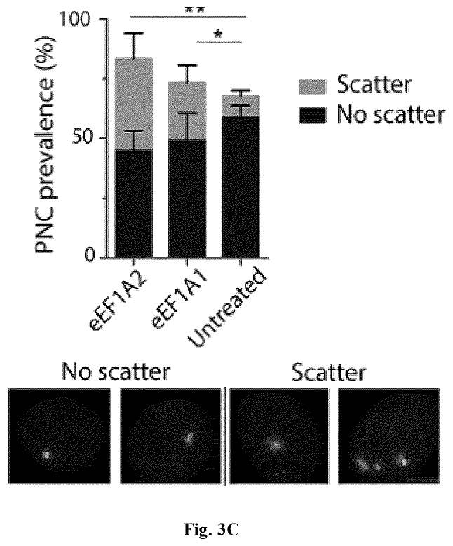

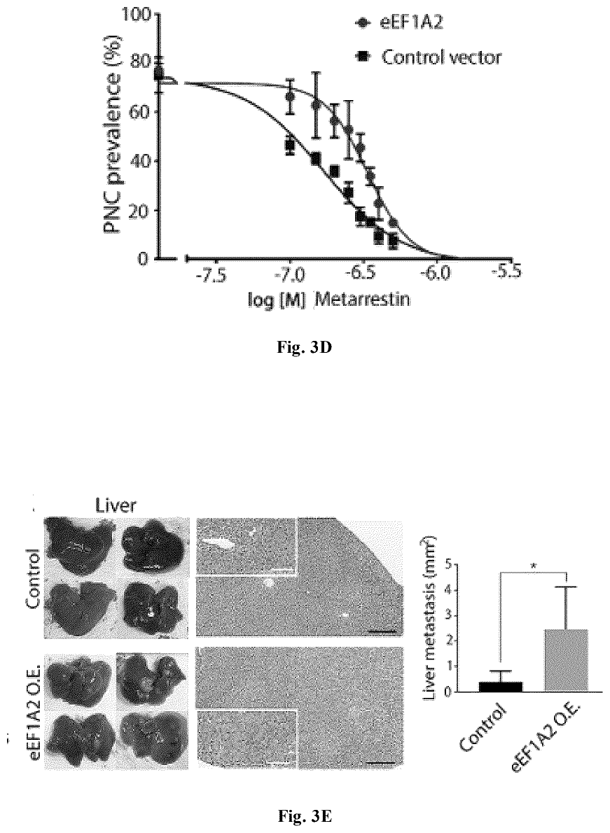

With respect to the metarrestin target eEF1A2, the biased expression of eEF1A2 in cancer cells over normal adult cells (except neuronal cells) was used to selectively target cancer cells [10, 11]. The interaction of metarrestin with eEF1A2 was investigated and revealed that metarrestin effectively outcompeted recombinant eEF1A2 from binding to an anchored biotinylated metarrestin-eEF1A2 complex (FIG. 3A) and metarrestin treatment stabilized eEF1A2 in a thermal stability assay using PC3M cell lysate (FIG. 3B). Increased eEF1A2 enhances PNC number per cell (FIG. 3C) and metastasis formation (FIG. 3E). Furthermore, eEF1A2 overexpression in PC3M cells increased the IC₅₀ of metarrestin for PNC disassembly in PC3M cells (FIG. 3D). Together, this data supports eEF1A2 as a critical target of metarrestin. Expression of eEF1A2 is associated with cancer metastasis and correlates with poor patient outcomes [35-44].

However, eEF1A2 is a multi-functional protein [45] that is not only involved in translational elongation of protein sets, but also involved in phosphatidylinositol signaling [46, 47], apoptosis [48], cytoskeletal modifications [49], participation in the heat shock response [50], and nuclear export [51, 52]. eEF1A2 is normally expressed in embryonic tissue and not in the majority of adult tissues. A notable exception is the eEF1A2 expression in neuronal brain cells and, to a lesser extent, in several isolated tissues (i.e. heart/skeletal muscle, pancreatic, adrenal, esophagus and seminal vesicle tissues) [53]. Not to be bound by theory, but it is believed that the eEF1A2 expression in an adult brain, combined with the known binding to metarrestin and observed CNS penetration of metarrestin, produces a scenario that explains the observed reversible seizure activity in large animals.

Thus, as already mentioned above, in order to increase the potency of metarrestin on tumor metastasis and to reduce CNS exposure, the compounds disclosed herein were prepared. It was surprising and unexpected to discover that the compounds disclosed herein exhibit potent eEF1A2 binding affinity while simultaneously demonstrating efficient degradation of the target protein (i.e., eEF1A2). Not to be bound by theory, but is it is believed that the eEF1A2 ligands bind to the eEF1A2 protein while at the same being able to modulate its degradation. These eEF1A2 ligands, pharmaceutical compositions containing these ligands, and methods of use thereof are described in more detail below.

### I. Definitions

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

As used herein, the term “mammal” includes, but is not limited to, the order Rodentia, such as mice, and the order Logomorpha, such as rabbits. It is preferred that the mammals are from the order Carnivora, including Felines (cats) and Canines (dogs). Itis more preferred that the mammals are from the order Artiodactyla, including Bovines (cows) and Swines (pigs) or of the order Perissodactyla, including Equines (horses). It is most preferred that the mammals are of the order Primates, Ceboids, or Simioids (monkeys) or of the order Anthropoids (humans and apes). An especially preferred mammal is the human. Furthermore, the subject can be the unborn offspring of any of the forgoing hosts, especially mammals (such as humans), in which case any screening of the subject or cells of the subject, or administration of compounds to the subject or cells of the subject, can be performed in utero.

As used herein, the term “effective amount” refers to the amount of a composition sufficient to effect beneficial or desired results in vivo and in vitro. An effective amount can be administered in one or more administrations, applications or dosages and is not intended to be limited to a particular formulation or administration route.

As used herein, the terms “administration” and “administering” refer to the act of giving a drug, prodrug, or other agent, or therapeutic treatment to a subject or in vivo, in vitro, or ex vivo cells, tissues, and organs (which can be normal or cancerous). Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) (e.g., a eEF1A2 ligand and one or more additional therapeutics) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “pharmaceutical composition” refers to the combination of an active agent with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption-delaying agents, disintegrants (e.g., potato starch or sodium starch glycolate), and the like. The compositions can also include stabilizers and preservatives. For examples of carriers, stabilizers and adjuvants, see, e.g., Martin, Remington's Pharmaceutical Sciences, 15th Ed., Mack Publ. Co., Easton, Pa. (1975), incorporated herein by reference in its entirety.

As used herein, the term “pharmaceutically acceptable salt” refers to any pharmaceutically acceptable salt (e.g., acid or base) of a compound of the present invention, which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent,” “preventing,” or “prevention” of any disease or disorder refers to the prophylactic treatment of the disease or disorder; or delaying the onset or progression of the disease or disorder.

As used herein, a subject is “in need of” a treatment if such subject would benefit biologically, medically or in quality of life from such treatment.

As used herein, the term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc.

As used herein, the term “anticancer agent” or“antineoplastic agent” refers to a therapeutic agent that is useful for treating or controlling the growth of cancerous cells.

### II. Compounds

Metarrestin shows selective inhibition of metastatic cancer cells in vivo, at least partially through disrupting the nucleolar structure. Metarrestin inhibits RNA polymerase (Pol) I transcription mediated in part by interacting with the translation elongation factor eEF1A2 [5]. In addition, metarrestin has been shown to interact with the translation elongation factor eEF1A2 protein directly.

Thus, the eEF1A2 ligands disclosed herein comprise a metarrestin moiety and an E3 ligase moiety connected by a linker (see FIG. 4). In some embodiments, the linker is aliphatic in nature. In some embodiments, the metarrestin moiety is metarrestin. In some embodiments, the metarrestin moiety is a metarrestin analogue where the side chain can be modified. Structure Activity Relationship (SAR) studies have shown that the side chain of the metarrestin moiety is important for altering the perinucleolar compartment (PNC) activity, i.e., PNC reducing activity. However, it would be understood that the effects of metarrestin and its analogues on the PNC are not limited to only RNA polymerases and/or translation elongation factor eEF1A2 but also include other cellular targets able to alter PNC activity. As such, in some embodiments, the metarrestin target(s) alter PNC activity, including PNC prevalence.

In some embodiments, the eEF1A2 ligands disclosed herein are a compound of Formula (A):

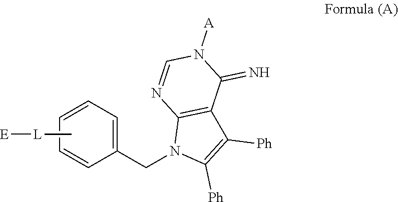

or an enantiomer, diastereomer, an enantiomeric mixture, a diastereomeric mixture or a pharmaceutically acceptable salt thereof; wherein:
A is selected from

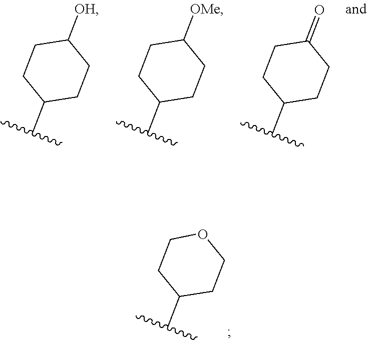 [SMILES: *C1CCC(O)CC1 ; *C1CCC(OC)CC1 ; *C1CCC(=O)CC1 ; *C1CCOCC1]

L is selected from

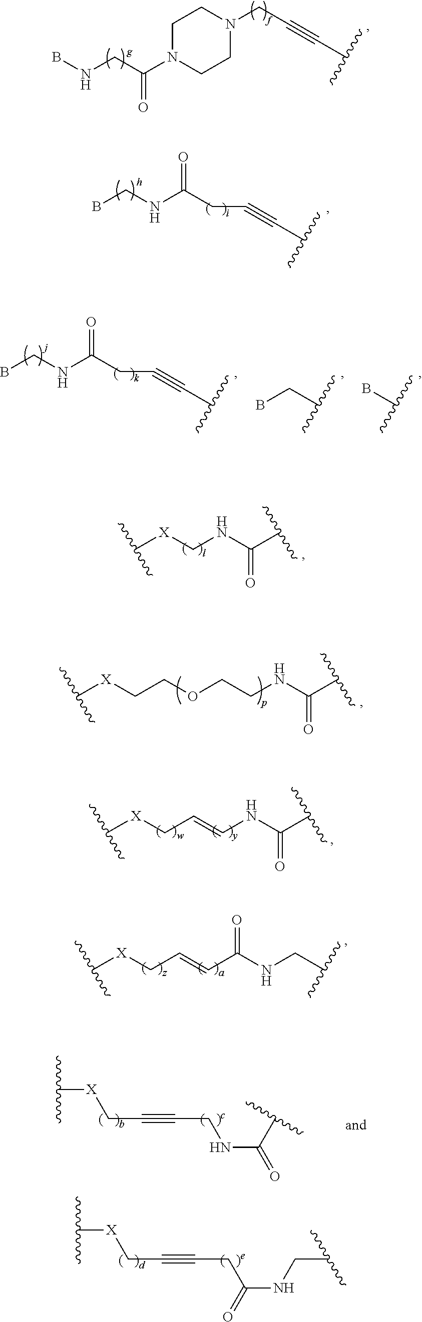

wherein a, b, c, d, e, f, g, h, i, j, k, l, p, w, y, and z are an integer independently selected from 0, 1, 2, 3, 4, 5, 6, 7, 8, 9, and 10;
wherein B is selected from

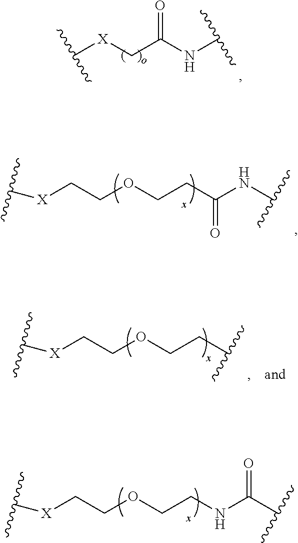

and a bond, wherein o and x are an integer independently selected from 0, 1, 2, 3, 4, 5, 6, 7, 8, 9, and X is selected from a bond, —CH₂—, —NH—, —NHC(═O)—, —C(═O)NH— and —O—; and
E is selected from

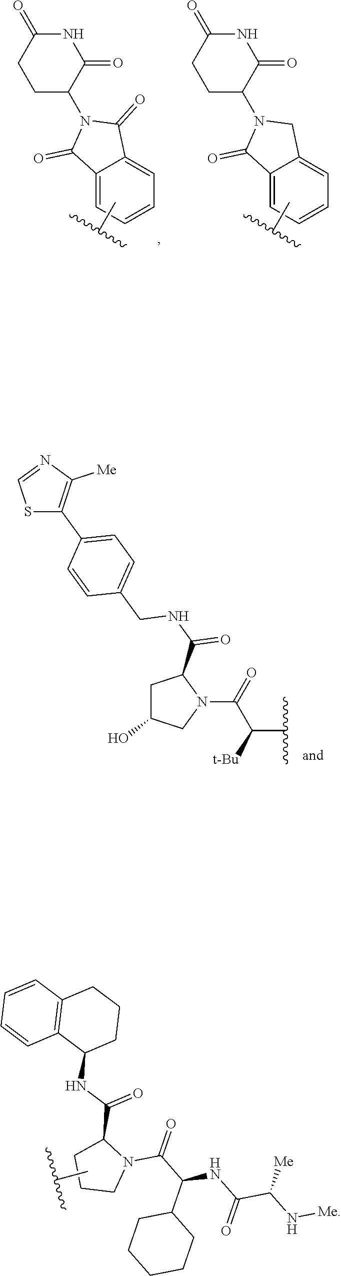

In some embodiments, a, b, c, d, e, f, g, h, i, j, k, l, p, w, y, and z are an integer independently selected from 0, 1, 2, 3, 4 and 5. In some embodiments, 1 is 0. In some embodiments, h is 0. In some embodiments, f is 1. In some embodiments, h, j, k, 1, and p are an integer independently selected from 1, 2, 3 and 4.

In some embodiments, X is —NH— or —NHC(═O)—.

In some embodiments, the eEF1A2 ligands disclosed herein are a compound of Formula (I):

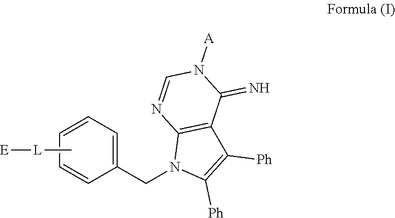

or an enantiomer, diastereomer, an enantiomeric mixture, a diastereomeric mixture or a pharmaceutically acceptable salt thereof; wherein:
A is selected from

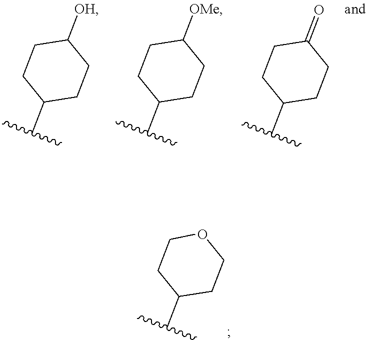 [SMILES: *C1CCC(O)CC1 ; *C1CCC(OC)CC1 ; *C1CCC(=O)CC1 ; *C1CCOCC1]

L is selected from

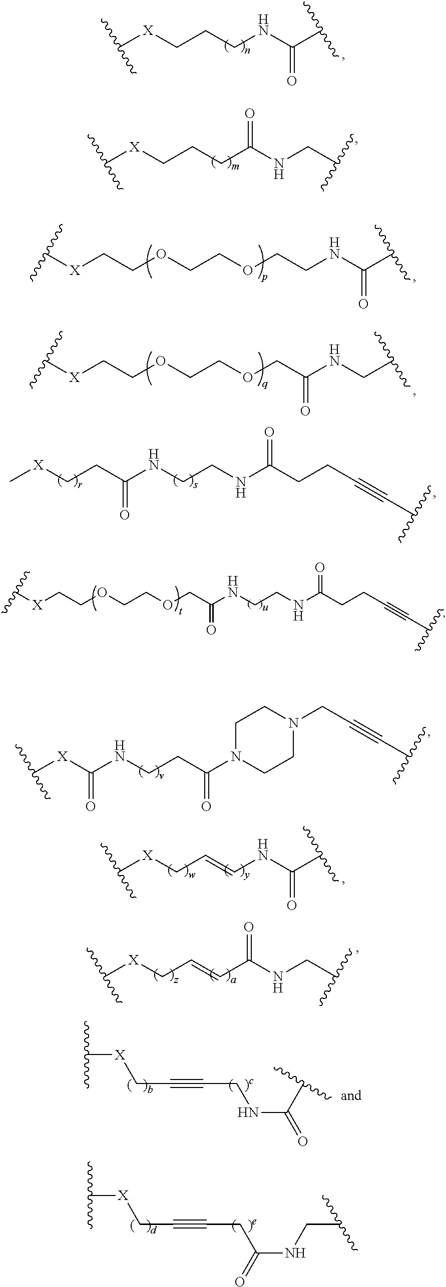

wherein a, b, c, d, e, n, m, p, q, r, s, t, u, v, w, y and z are an integer independently selected from 0, 1, 2, 3, 4, 5, 6, 7, 8, 9, and 10, and X is selected from —CH₂—, —NH—, and —O—; and
E is selected from

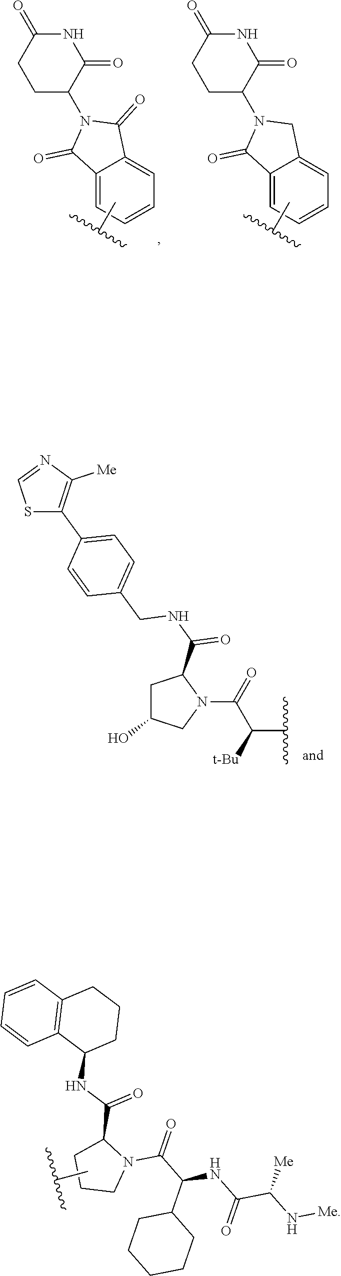

In some embodiments, X is selected from —NH—, and —O—.

In some embodiments, a, b, c, d, e, n, m, p, q, r, s, t, u, v, w, y and z are an integer independently selected from 1, 2, 3, 4, 5, 6, 7, 8, 9 and 10. In some embodiments, a, b, c, d, e, n, m, p, q, r, s, t, u, v, w, y and z are an integer independently selected from 1, 2, 3, 4, 5, 6, 7 and 8.

In some embodiments, X is selected from —NH—, and —O—, and a, b, c, d, e, n, m, p, q, r, s, t, u, v, w, y and z are an integer independently selected from 1, 2, 3, 4, 5, 6, 7 and 8.

In some embodiments, E is

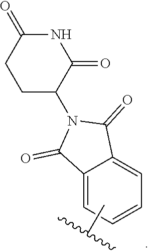

In some embodiments, the compound disclosed herein comprises a compound of Formula (II):

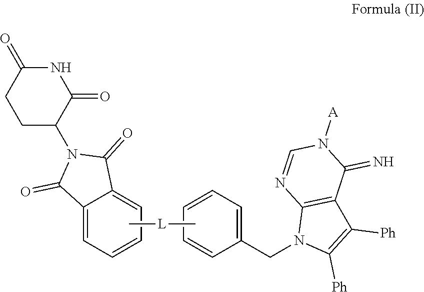

or an enantiomer, diastereomer, an enantiomeric mixture, a diastereomeric mixture or a pharmaceutically acceptable salt thereof; wherein:
A is selected from

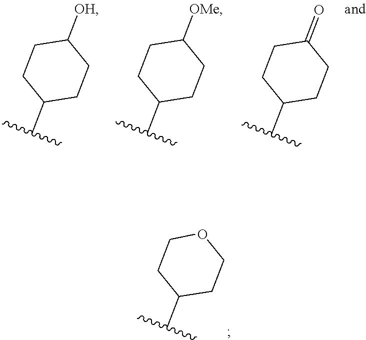 [SMILES: *C1CCC(O)CC1 ; *C1CCC(OC)CC1 ; *C1CCC(=O)CC1 ; *C1CCOCC1]

and
L is selected from

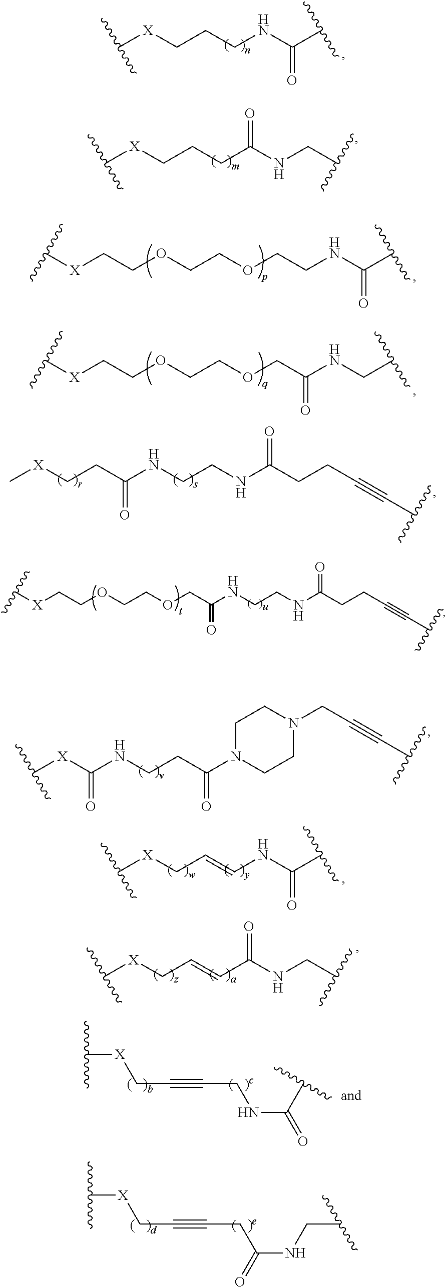

wherein a, b, c, d, e, n, m, p, q, r, s, t, u, v, w, y and z are an integer independently selected from 1, 2, 3, 4, 5, 6, 7, and 8, and X is selected from —CH₂—, —NH—, and —O—.

In some embodiments, X is —NH—, or —O—.

In some embodiments, A is

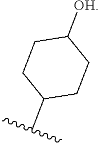 [SMILES: *C1CCC(O)CC1]

In some embodiments, X is —NH—, or —O—; and A is

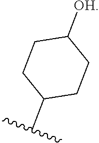 [SMILES: *C1CCC(O)CC1]

In some embodiments, p, q, and t are an integer independently selected from 1, 2, 3, and 4. In some embodiments, X is —NH—, or —O—; and A is

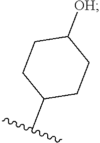 [SMILES: *C1CCC(O)CC1]

and
p, q, and t are an integer independently selected from 1, 2, 3, and 4.

In some embodiments, n, m, r, s, u, v, w, y, z, a, b, d, and e are an integer independently selected from 1, 2, 3, 4, 5, 6, 7 and 8.

In some embodiments, X is —NH—, or —O—; and A is

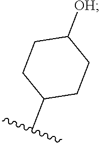 [SMILES: *C1CCC(O)CC1]

and
n, m, r, s, u, v, w, y, z, a, b, d, and e are an integer independently selected from 1, 2, 3, 4, 5, 6, 7 and 8.

In some embodiments, L is selected from

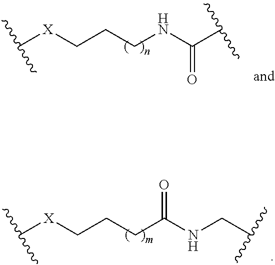

In some embodiments, the compound disclosed herein comprises a compound of Formula (III):

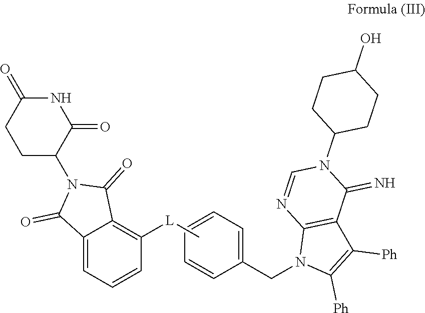

or an enantiomer, diastereomer, an enantiomeric mixture, a diastereomeric mixture or a pharmaceutically acceptable salt thereof; wherein L is selected from

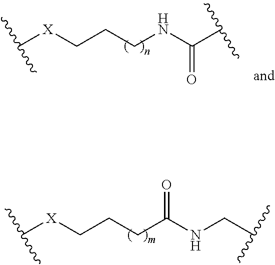

wherein n and m are an integer independently selected from 1, 2, 3, 4, 5, 6, 7, and 8; and X is selected from —NH—, and —O—.

In some embodiments, n and m are an integer independently selected from 5, 6, 7, and 8.

In some embodiments, n and m are 5.

In some embodiments, n and m are an integer independently selected from 5, 6, 7, and 8; and X is —NH—.

In some embodiments, L is

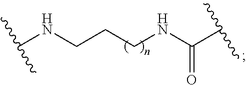

and n is an integer independently selected from 5, 6, 7, and 8.

In some embodiments, L is

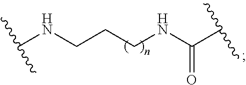

and n is 5.

In some embodiments, the compound of Formula (I), (II) or (III) is

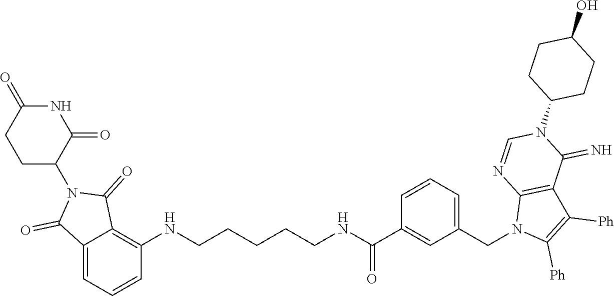 [SMILES: N=c1c2c(-c3ccccc3)c(-c3ccccc3)n(Cc3cccc(C(=O)NCCCCCNc4cccc5c4C(=O)N(C4CCC(=O)NC4=O)C5=O)c3)c2ncn1[C@H]1CC[C@H](O)CC1]

or a pharmaceutically acceptable salt thereof.

In some embodiments, L is

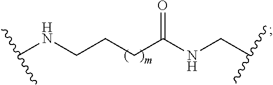

and m is an integer independently selected from 5, 6, 7, and 8.

In some embodiments, L is

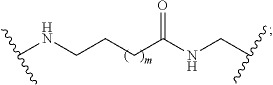

and m is 5.

In some embodiments, the compound of Formula (I), (II) or (III) is

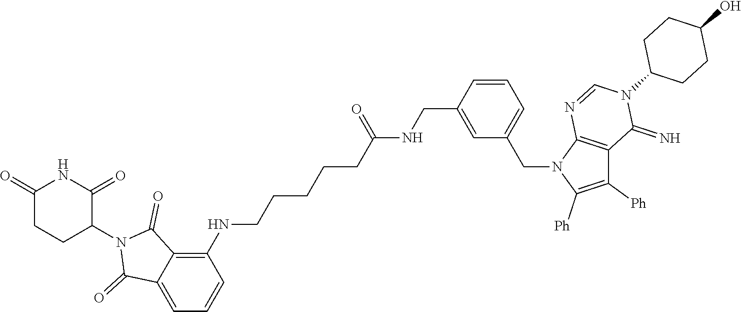 [SMILES: N=c1c2c(-c3ccccc3)c(-c3ccccc3)n(Cc3cccc(CNC(=O)CCCCCNc4cccc5c4C(=O)N(C4CCC(=O)NC4=O)C5=O)c3)c2ncn1[C@H]1CC[C@H](O)CC1]

or a pharmaceutically acceptable salt thereof.

Another aspect of the current disclosure is directed to eEF1A2 ligands, which are a compound of Formula (B):

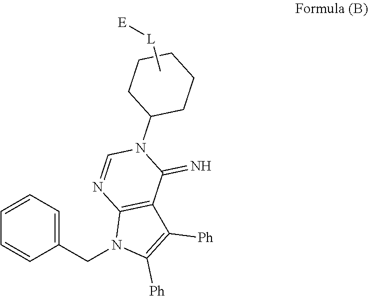

or an enantiomer, diastereomer, an enantiomeric mixture, a diastereomeric mixture or a pharmaceutically acceptable salt thereof; wherein:
L is selected from

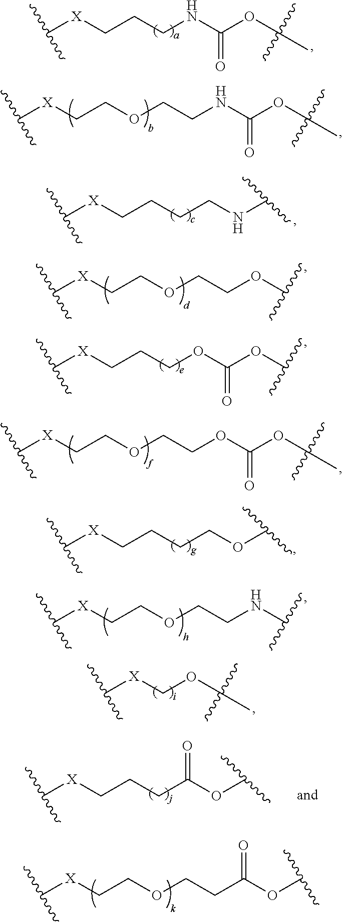

wherein a, b, c, d, e, f, g, h, i, j, and k are an integer independently selected from 0, 1, 2, 3, 4, 5, 6, 7, and 8, and X is selected from a bond —CH₂—, —NH—, and —O—, and
E is selected from

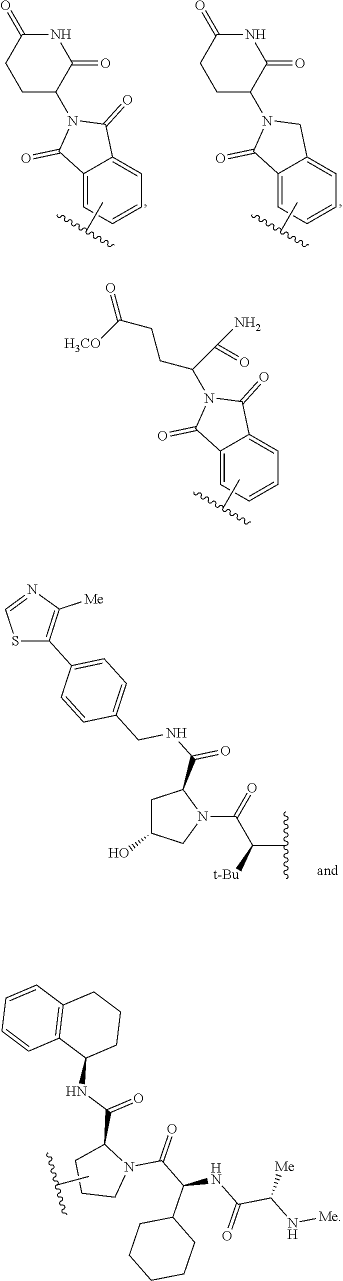

In some embodiments, X is selected from —NH— and —O—.

In some embodiments, a, b, c, d, e, f, g, h, i, j, and k are an integer independently selected from 0, 1, 2, 3, 4, and 5.

In some embodiments, X is selected from —NH— and —O— and a, b, c, d, e, f, g, h, i, j, and k are an integer independently selected from 0, 1, 2, 3, 4, and 5.

In some embodiments, E is selected from

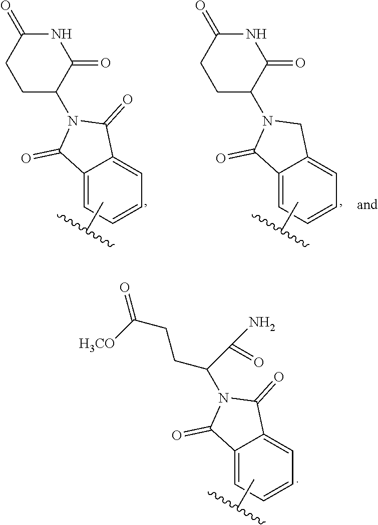

In some embodiments, the compound disclosed herein comprises a compound of formula IV:

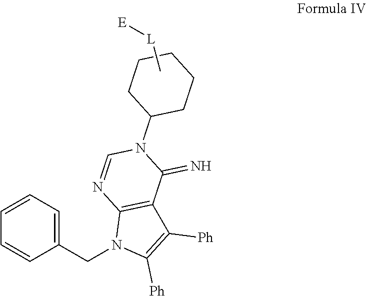

or an enantiomer, diastereomer, an enantiomeric mixture, a diastereomeric mixture or a pharmaceutically acceptable salt thereof; wherein:
L is selected from

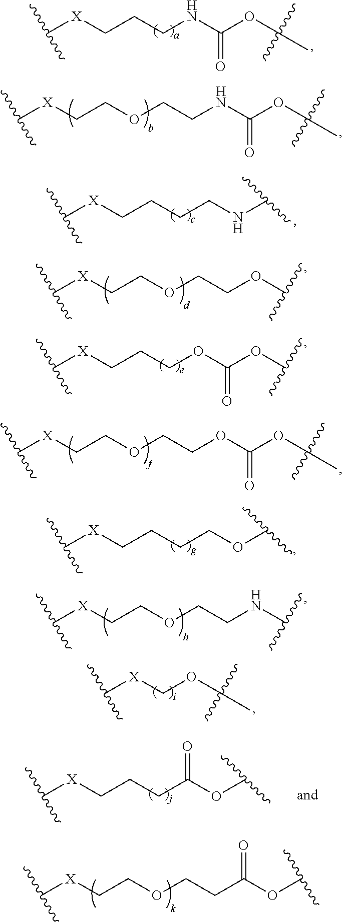

wherein g, d, i, j, and k are an integer independently selected from 0, 1, 2, 3, 4, 5, 6, 7, and 8, and X is selected from a bond —CH₂—, —NH—, and —O—, and
E is selected from

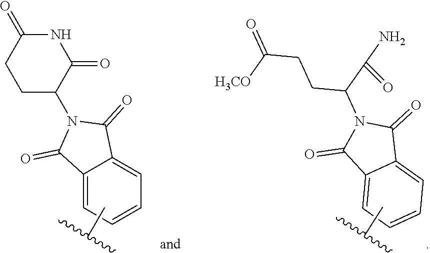

In some embodiments, X is selected from —NH— and —O—.

In some embodiments, L is selected from

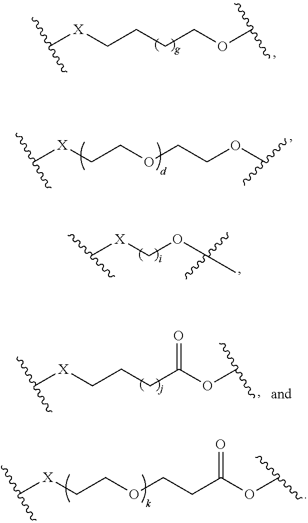

In some embodiments, g, d, i, j, and k are an integer independently selected from 1, 2, 3, 4, and 5.

In some embodiments, L is selected from

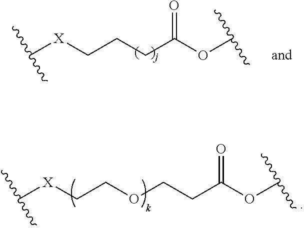

In some embodiments, X is selected from —NH— and —O— and L is selected from

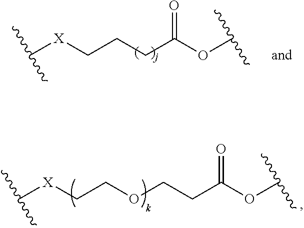

wherein j and k, are an integer independently selected from 0, 1, 2, 3, 4, and 5.

In some embodiments, X is —NH—.

In some embodiments, X is —NH— and L is

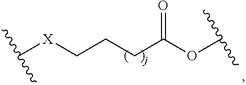

wherein j is an integer selected from 1, 2, or 3.

In some embodiments, E is

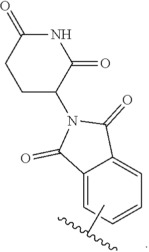

In some embodiments, E is

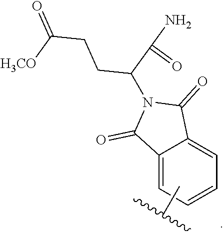

In some embodiments, X is —NH— and L is

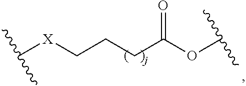

wherein j is an integer selected from 1 or 3, and E is

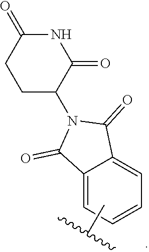

In some embodiments, X is —NH— and L is

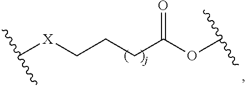

wherein j is 1, and E is

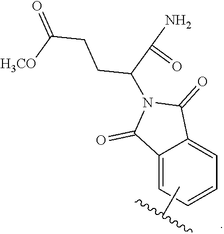

In some embodiments, X is —NH— and L is

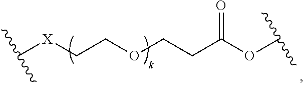

wherein k is an integer selected from 1, 2, or 3, and E is

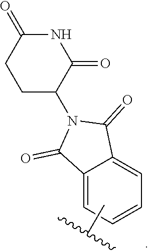

In some embodiments, X is —NH— and L is

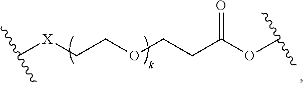

wherein k is 3, and E is

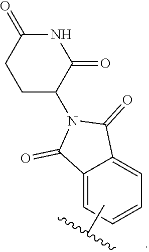

In some embodiments, the compound of Formula (B) or (IV) is selected from

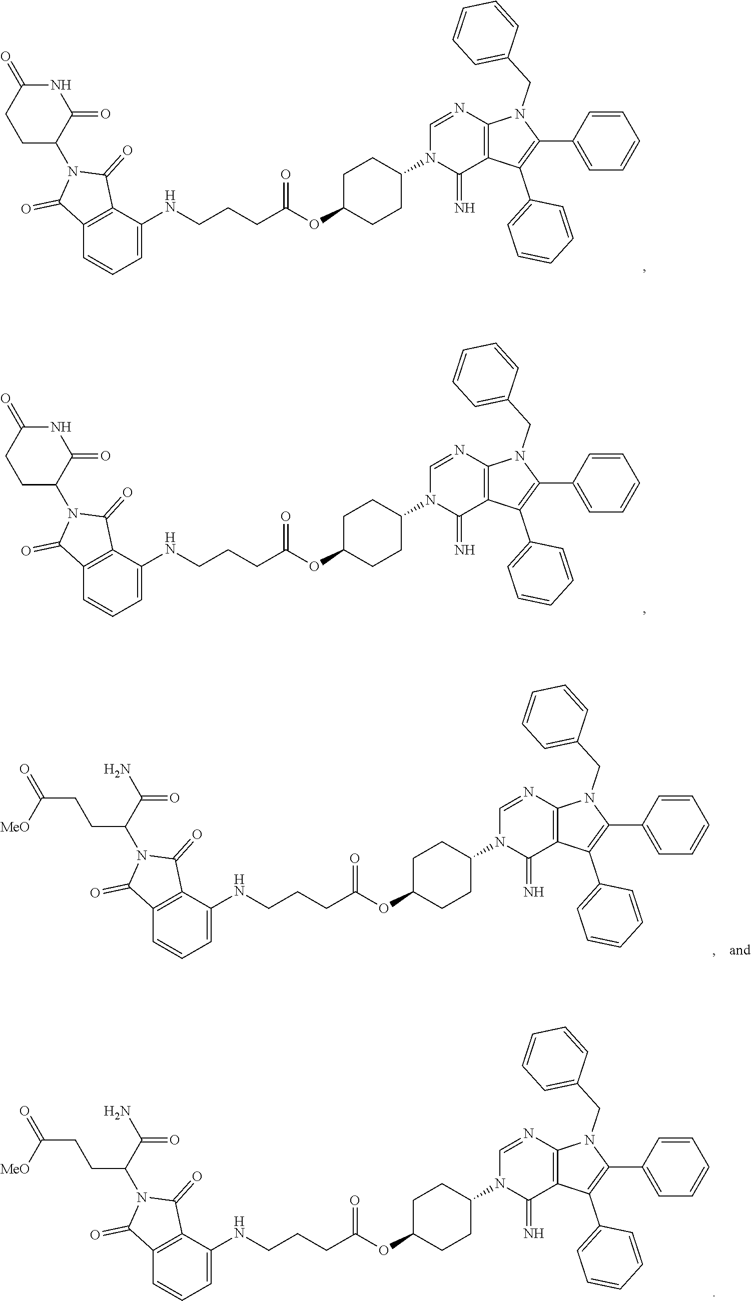 [SMILES: N=c1c2c(-c3ccccc3)c(-c3ccccc3)n(Cc3ccccc3)c2ncn1[C@H]1CC[C@H](OC(=O)CCCNc2cccc3c2C(=O)N(C2CCC(=O)NC2=O)C3=O)CC1 ; N=c1c2c(-c3ccccc3)c(-c3ccccc3)n(Cc3ccccc3)c2ncn1[C@H]1CC[C@H](OC(=O)CCCNc2cccc3c2C(=O)N(C2CCC(=O)NC2=O)C3=O)CC1 ; COC(=O)CCC(C(N)=O)N1C(=O)c2cccc(NCCCC(=O)O[C@H]3CC[C@H](n4cnc5c(c(-c6ccccc6)c(-c6ccccc6)n5Cc5ccccc5)c4=N)CC3)c2C1=O ; COC(=O)CCC(C(N)=O)N1C(=O)c2cccc(NCCCC(=O)O[C@H]3CC[C@H](n4cnc5c(c(-c6ccccc6)c(-c6ccccc6)n5Cc5ccccc5)c4=N)CC3)c2C1=O]

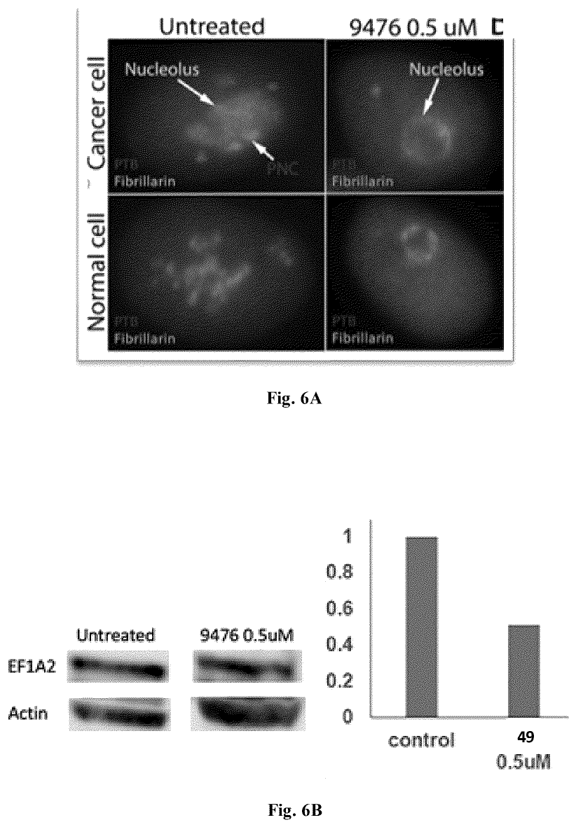

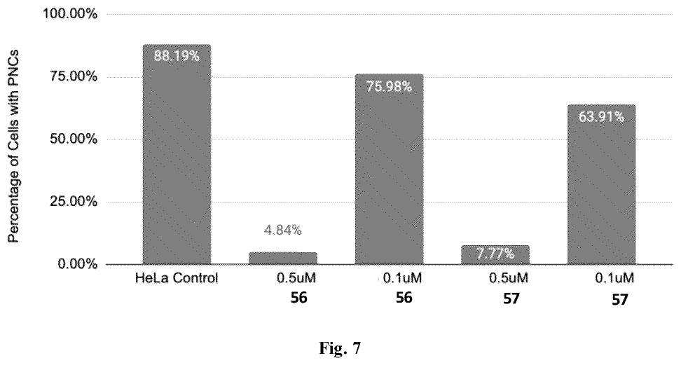

Such exemplary compounds as disclosed in the above embodiments exhibit significantly improved potency compared to metarrestin (FIG. 6A). For example, Compound 46 is threefold more potent than metarrestin and the structurally related Compound 49 is 14-fold more potent, as is shown in the table below. The data in the below table further shows that both compounds demonstrate improved potency for PNC inhibition (i.e., IC₅₀ inhibiting PNC in cancer cells, IC₅₀ disrupting nucleoli in normal cells, and the ratio of the two).

| | IC50 | | IC50 |
|---|---|---|---|
| | nucleoli.normal | IC50 PNC | nucleoli.normal/IC50 |
| Compounds | μM RPE1 | μM HeLa | PNC.cancer |
| Compound 49 | 0.1 | 0.025 | 4 |
| Compound 46 | 0.47 | 0.11 | 4.27 |
| Metarrestin | 2.5 | 0.36 | 6.25 |

Preliminary Western blot analyses demonstrated that treatment with Compound 49 at 0.5 M for 20 hours significantly reduced eEF1A2 protein levels (FIG. 6C). While metarrestin binds eEF1A2, it does not reduce the protein levels under similar treatment conditions [5]. Thus, this result indicates that the derivatives act as bifunctional degraders and further supports the direct binding of metarrestin with eEF1A2.

Thus, in some embodiments, the compound of any one of Formula (A), (B), (I), (II), (III) and (IV) may be selected from the compound listed in Table 1. Compounds of Formula (A), (B) (I), (II), (III) and (IV) that are not listed in Table 1 are also within the scope herein.

TABLE 1

| Com- | |
|---|---|
| pound | |
| # | Structure |
| 1 | 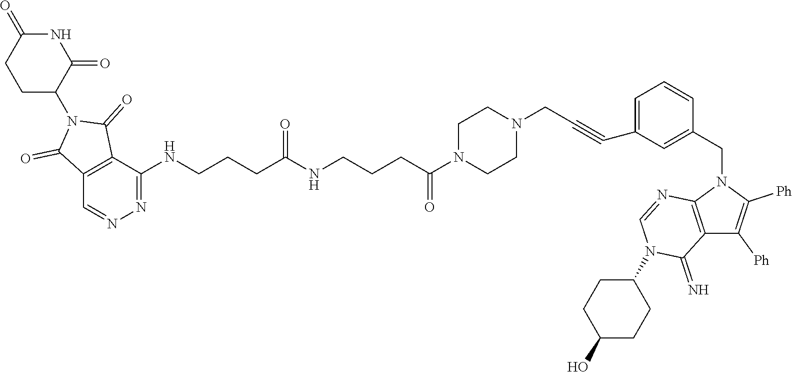 [SMILES: N=c1c2c(-c3ccccc3)c(-c3ccccc3)n(Cc3cccc(C#CCN4CCN(C(=O)CCCNC(=O)CCCNc5nncc6c5C(=O)N(C5CCC(=O)NC5=O)C6=O)CC4)c3)c2ncn1[C@H]1CC[C@H](O)CC1] |
| 2 | 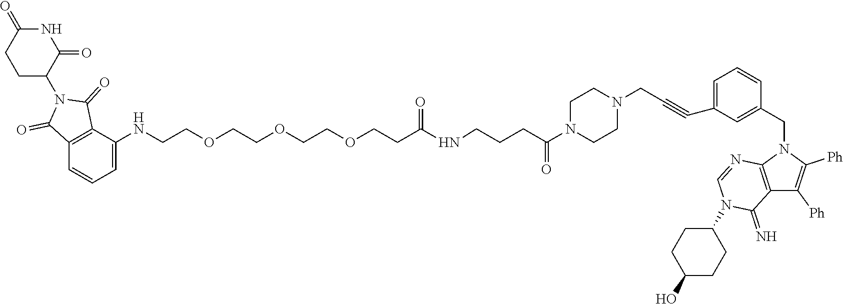 [SMILES: N=c1c2c(-c3ccccc3)c(-c3ccccc3)n(Cc3cccc(C#CCN4CCN(C(=O)CCCNC(=O)CCOCCOCCOCCNc5cccc6c5C(=O)N(C5CCC(=O)NC5=O)C6=O)CC4)c3)c2ncn1[C@H]1CC[C@H](O)CC1] |
| 3 | 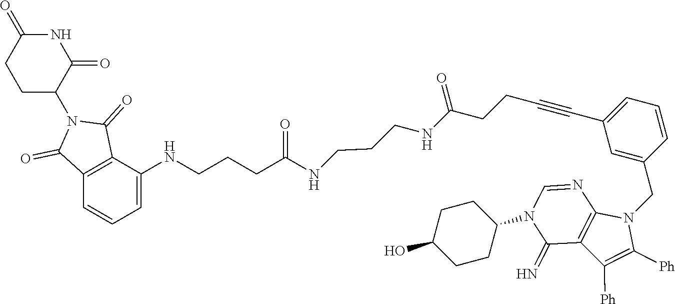 [SMILES: N=c1c2c(-c3ccccc3)c(-c3ccccc3)n(Cc3cccc(C#CCCC(=O)NCCCNC(=O)CCCNc4cccc5c4C(=O)N(C4CCC(=O)NC4=O)C5=O)c3)c2ncn1[C@H]1CC[C@H](O)CC1] |
| 4 | 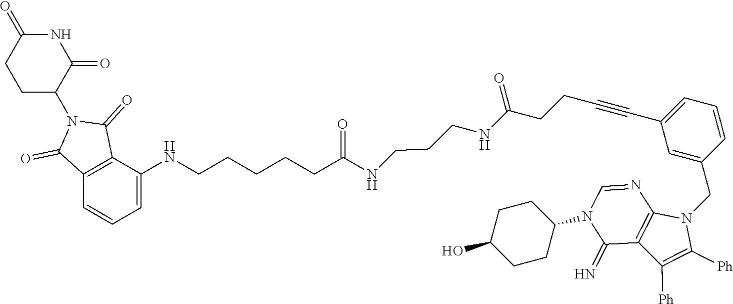 [SMILES: N=c1c2c(-c3ccccc3)c(-c3ccccc3)n(Cc3cccc(C#CCCC(=O)NCCCNC(=O)CCCCCNc4cccc5c4C(=O)N(C4CCC(=O)NC4=O)C5=O)c3)c2ncn1[C@H]1CC[C@H](O)CC1] |
| 5 | 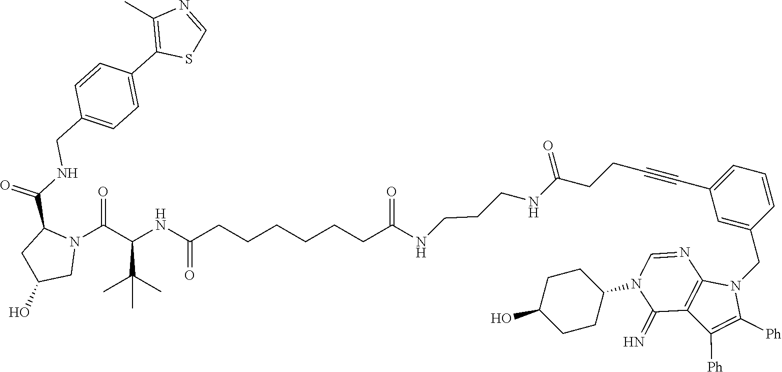 [SMILES: Cc1ncsc1-c1ccc(CNC(=O)[C@@H]2C[C@@H](O)CN2C(=O)[C@@H](NC(=O)CCCCCCC(=O)NCCCNC(=O)CCC#Cc2cccc(Cn3c(-c4ccccc4)c(-c4ccccc4)c4c(=N)n([C@H]5CC[C@H](O)CC5)cnc43)c2)C(C)(C)C)cc1] |
| 6 | 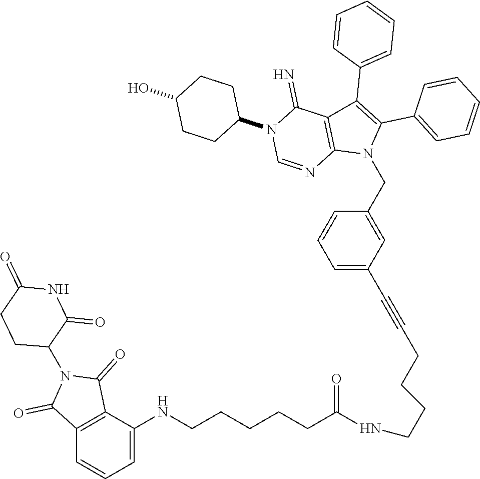 |
| 7 | 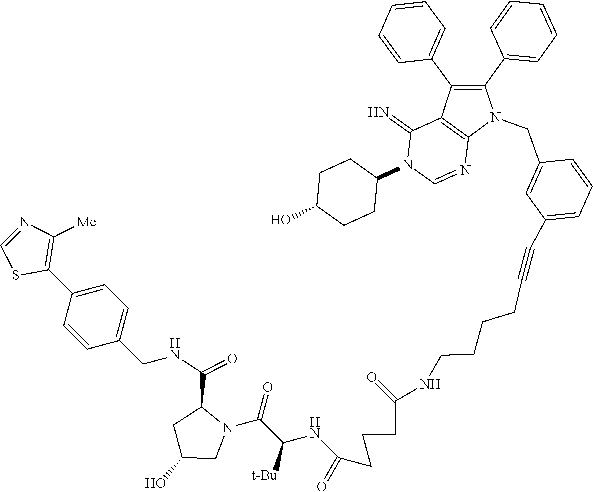 |
| 8 | 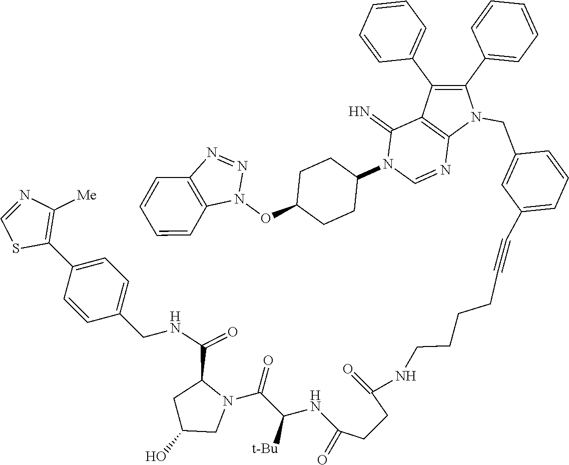 |
| 9 | 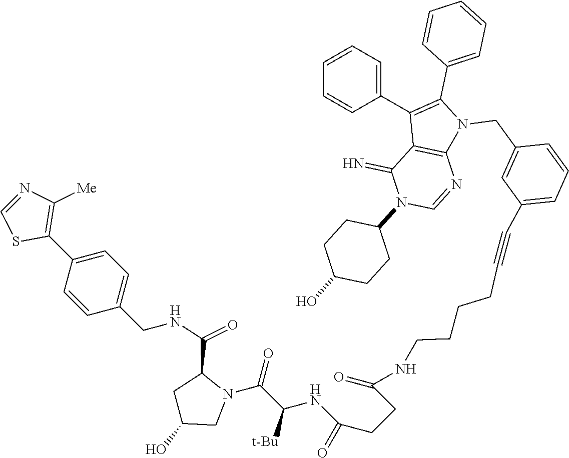 |
| 10 | 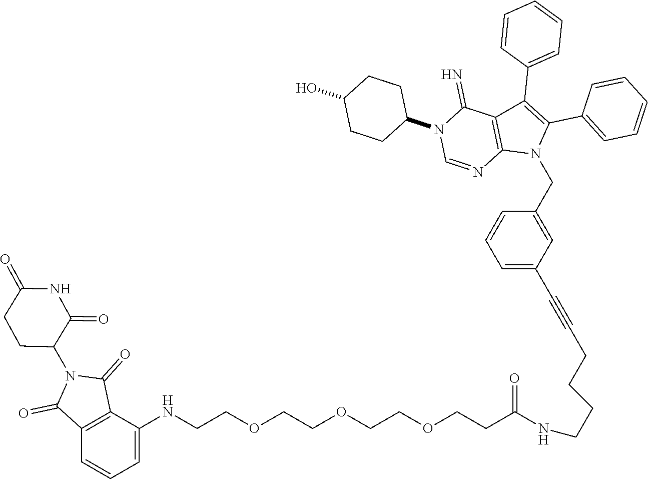 |
| 11 | 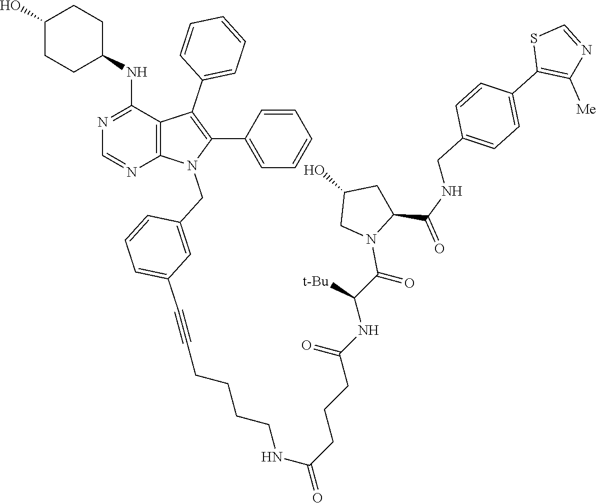 [SMILES: Cc1ncsc1-c1ccc(CNC(=O)[C@@H]2C[C@@H](O)CN2C(=O)[C@@H](NC(=O)CCCC(=O)NCCCCC#Cc2cccc(Cn3c(-c4ccccc4)c(-c4ccccc4)c4c(N[C@H]5CC[C@H](O)CC5)ncnc43)c2)C(C)(C)C)cc1] |
| 12 | 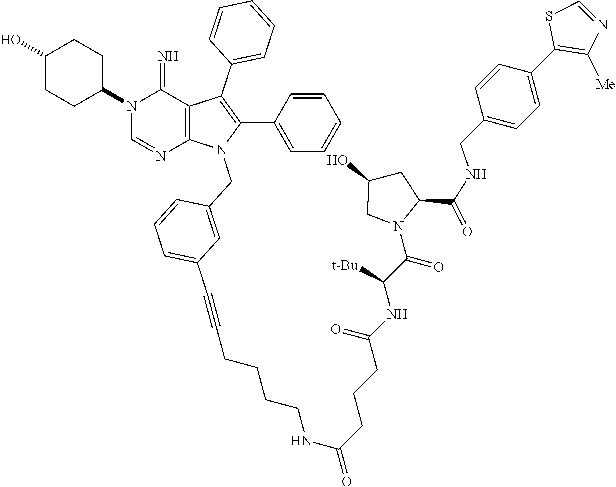 |
| 13 | 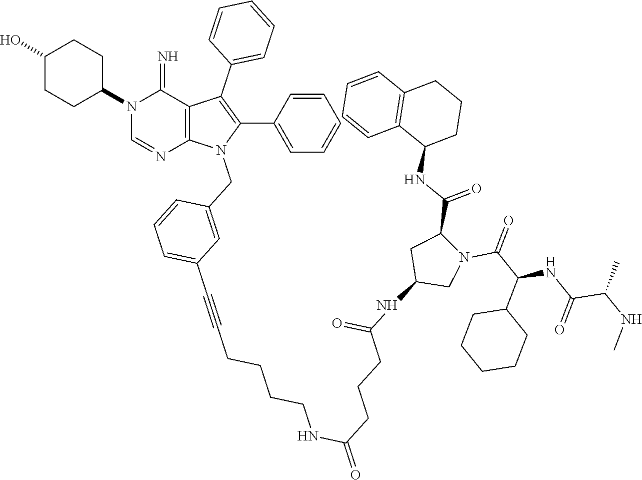 [SMILES: CN[C@@H](C)C(=O)N[C@H](C(=O)N1C[C@@H](NC(=O)CCCC(=O)NCCCCC#Cc2cccc(Cn3c(-c4ccccc4)c(-c4ccccc4)c4c(=N)n([C@H]5CC[C@H](O)CC5)cnc43)c2)C[C@H]1C(=O)N[C@@H]1CCCc2ccccc21)C1CCCCC1] |
| 14 | 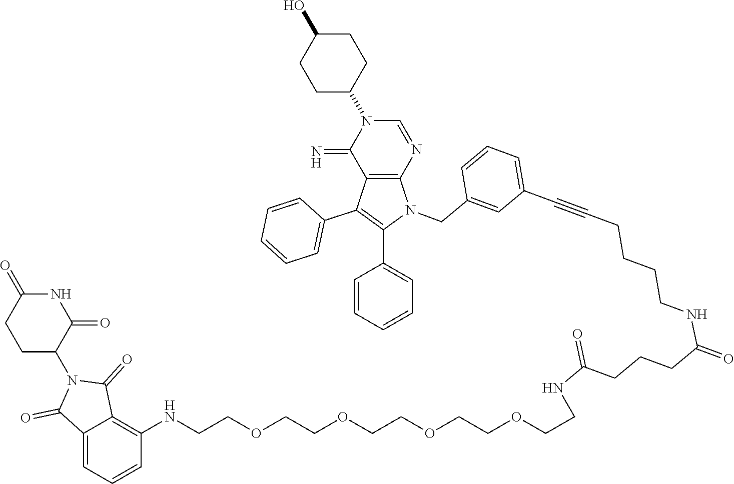 |
| 15 | 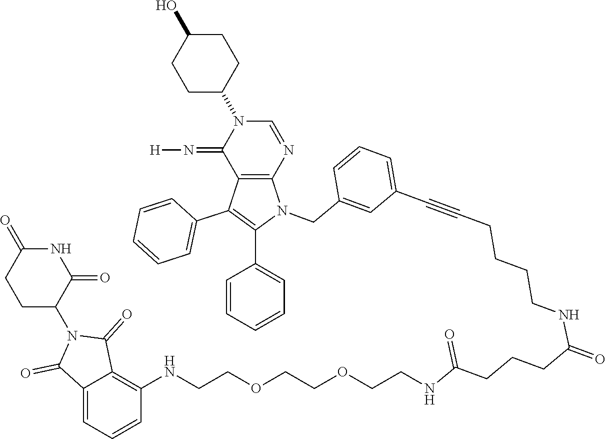 |
| 16 | 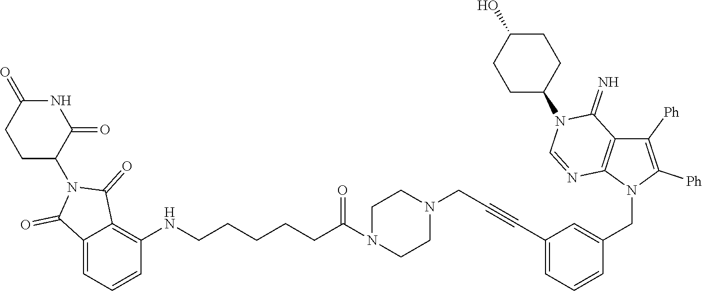 [SMILES: N=c1c2c(-c3ccccc3)c(-c3ccccc3)n(Cc3cccc(C#CCN4CCN(C(=O)CCCCCNc5cccc6c5C(=O)N(C5CCC(=O)NC5=O)C6=O)CC4)c3)c2ncn1[C@H]1CC[C@H](O)CC1] |
| 17 | 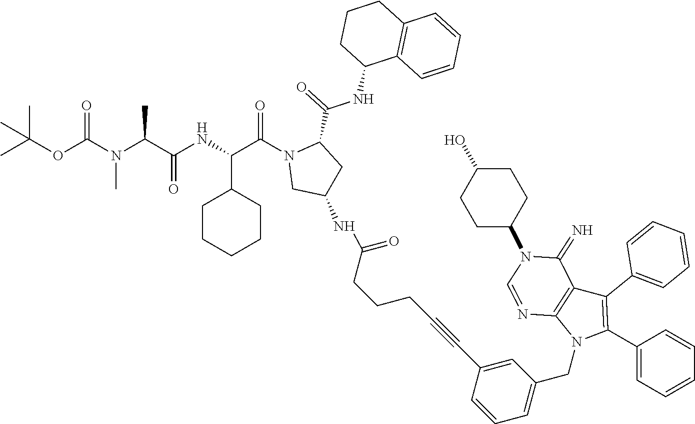 |
| 18 | 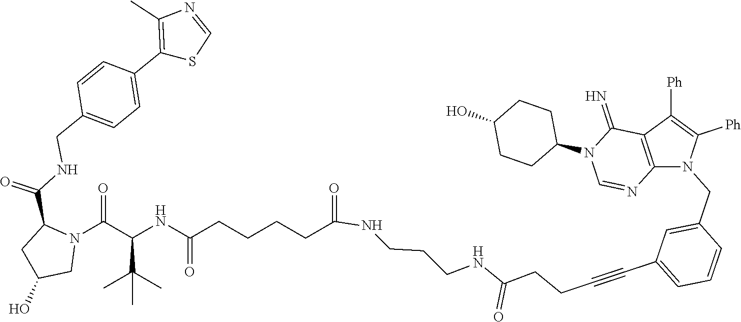 [SMILES: Cc1ncsc1-c1ccc(CNC(=O)[C@@H]2C[C@@H](O)CN2C(=O)[C@@H](NC(=O)CCCCC(=O)NCCCNC(=O)CCC#Cc2cccc(Cn3c(-c4ccccc4)c(-c4ccccc4)c4c(=N)n([C@H]5CC[C@H](O)CC5)cnc43)c2)C(C)(C)C)cc1] |
| 19 | 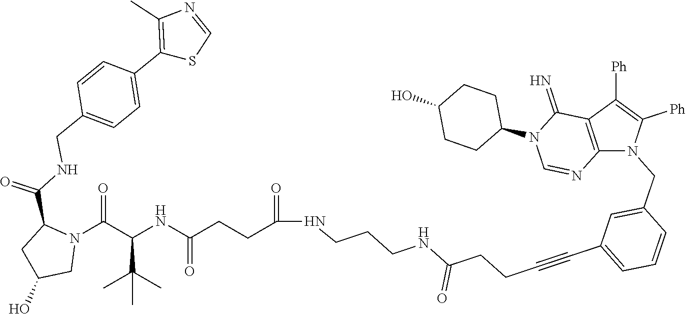 [SMILES: Cc1ncsc1-c1ccc(CNC(=O)[C@@H]2C[C@@H](O)CN2C(=O)[C@@H](NC(=O)CCC(=O)NCCCNC(=O)CCC#Cc2cccc(Cn3c(-c4ccccc4)c(-c4ccccc4)c4c(=N)n([C@H]5CC[C@H](O)CC5)cnc43)c2)C(C)(C)C)cc1] |
| 20 | 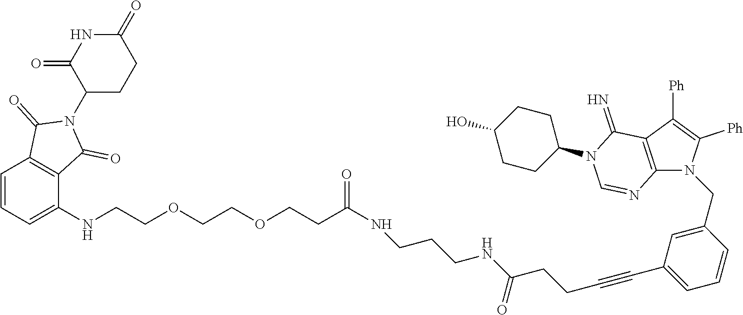 [SMILES: N=c1c2c(-c3ccccc3)c(-c3ccccc3)n(Cc3cccc(C#CCCC(=O)NCCCNC(=O)CCOCCOCCNc4cccc5c4C(=O)N(C4CCC(=O)NC4=O)C5=O)c3)c2ncn1[C@H]1CC[C@H](O)CC1] |
| 21 | 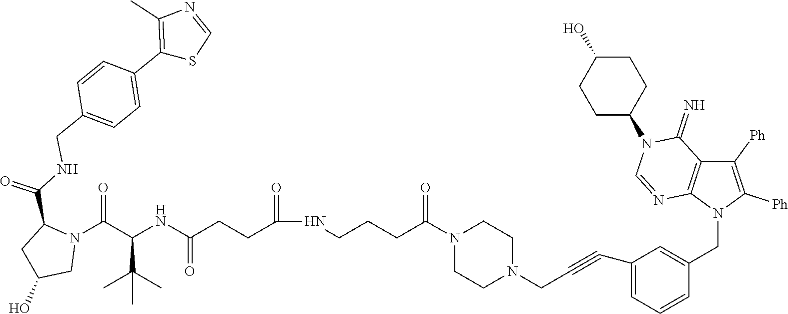 [SMILES: Cc1ncsc1-c1ccc(CNC(=O)[C@@H]2C[C@@H](O)CN2C(=O)[C@@H](NC(=O)CCC(=O)NCCCC(=O)N2CCN(CC#Cc3cccc(Cn4c(-c5ccccc5)c(-c5ccccc5)c5c(=N)n([C@H]6CC[C@H](O)CC6)cnc54)c3)CC2)C(C)(C)C)cc1] |
| 22 | 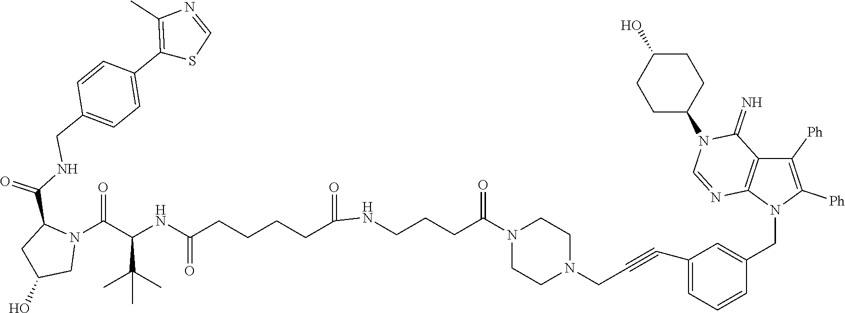 [SMILES: Cc1ncsc1-c1ccc(CNC(=O)[C@@H]2C[C@@H](O)CN2C(=O)[C@@H](NC(=O)CCCCC(=O)NCCCC(=O)N2CCN(CC#Cc3cccc(Cn4c(-c5ccccc5)c(-c5ccccc5)c5c(=N)n([C@H]6CC[C@H](O)CC6)cnc54)c3)CC2)C(C)(C)C)cc1] |
| 23 | 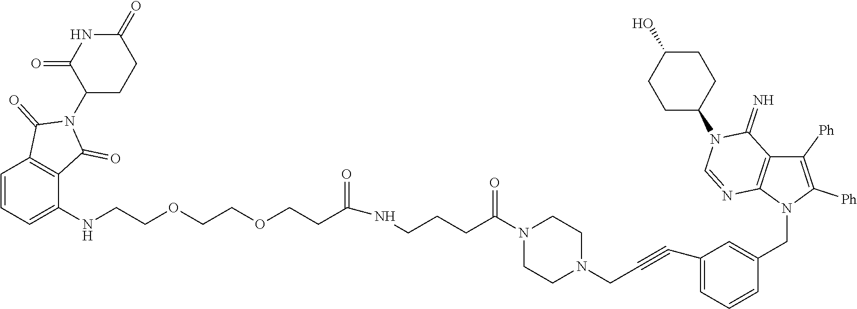 [SMILES: N=c1c2c(-c3ccccc3)c(-c3ccccc3)n(Cc3cccc(C#CCN4CCN(C(=O)CCCNC(=O)CCOCCOCCNc5cccc6c5C(=O)N(C5CCC(=O)NC5=O)C6=O)CC4)c3)c2ncn1[C@H]1CC[C@H](O)CC1] |
| 24 | 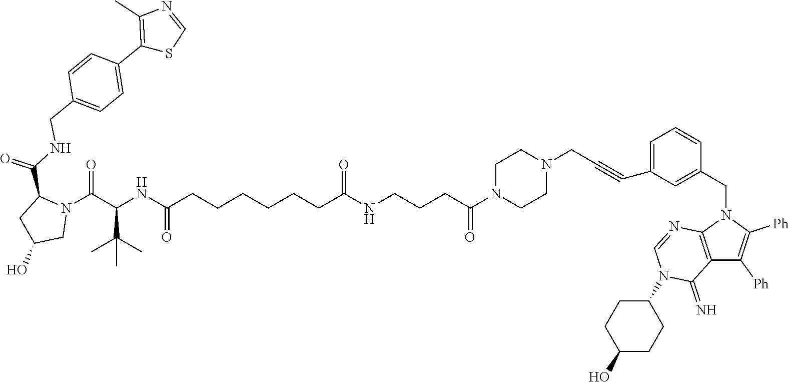 [SMILES: Cc1ncsc1-c1ccc(CNC(=O)[C@@H]2C[C@@H](O)CN2C(=O)[C@@H](NC(=O)CCCCCCC(=O)NCCCC(=O)N2CCN(CC#Cc3cccc(Cn4c(-c5ccccc5)c(-c5ccccc5)c5c(=N)n([C@H]6CC[C@H](O)CC6)cnc54)c3)CC2)C(C)(C)C)cc1] |
| 25 | 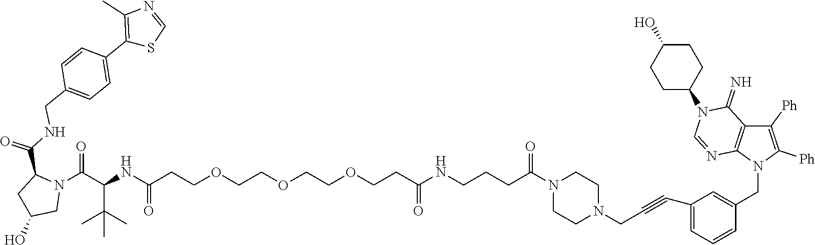 [SMILES: Cc1ncsc1-c1ccc(CNC(=O)[C@@H]2C[C@@H](O)CN2C(=O)[C@@H](NC(=O)CCOCCOCCOCCC(=O)NCCCC(=O)N2CCN(CC#Cc3cccc(Cn4c(-c5ccccc5)c(-c5ccccc5)c5c(=N)n([C@H]6CC[C@H](O)CC6)cnc54)c3)CC2)C(C)(C)C)cc1] |
| 26 | 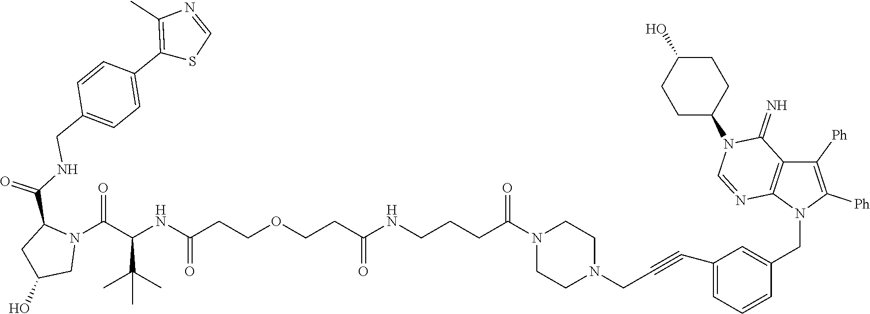 [SMILES: Cc1ncsc1-c1ccc(CNC(=O)[C@@H]2C[C@@H](O)CN2C(=O)[C@@H](NC(=O)CCOCCC(=O)NCCCC(=O)N2CCN(CC#Cc3cccc(Cn4c(-c5ccccc5)c(-c5ccccc5)c5c(=N)n([C@H]6CC[C@H](O)CC6)cnc54)c3)CC2)C(C)(C)C)cc1] |
| 27 | 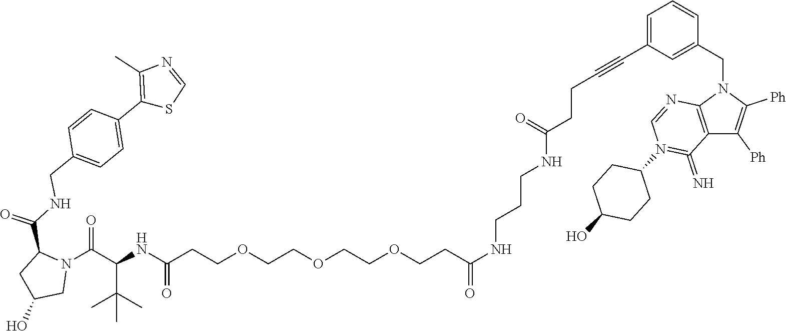 |
| 28 | 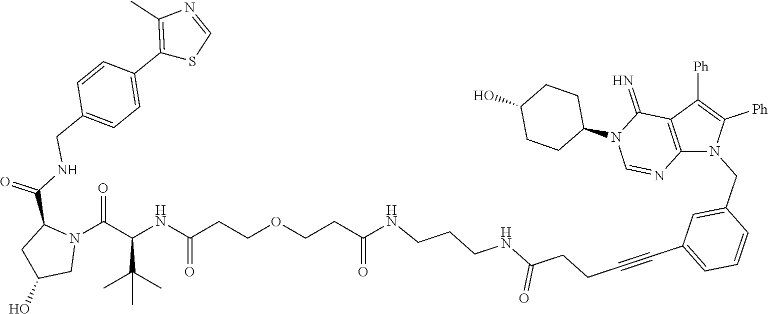 [SMILES: Cc1ncsc1-c1ccc(CNC(=O)[C@@H]2C[C@@H](O)CN2C(=O)[C@@H](NC(=O)CCOCCC(=O)NCCCNC(=O)CCC#Cc2cccc(Cn3c(-c4ccccc4)c(-c4ccccc4)c4c(=N)n([C@H]5CC[C@H](O)CC5)cnc43)c2)C(C)(C)C)cc1] |
| 29 | 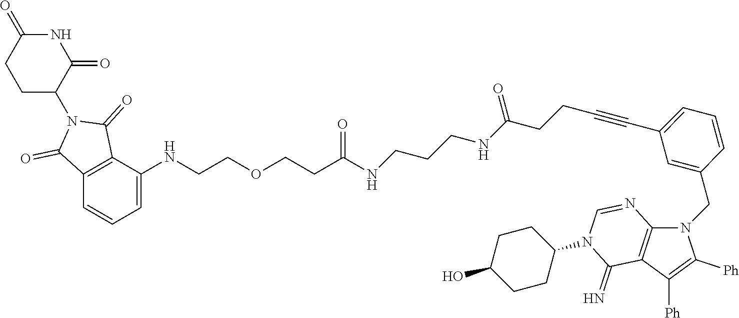 [SMILES: N=c1c2c(-c3ccccc3)c(-c3ccccc3)n(Cc3cccc(C#CCCC(=O)NCCCNC(=O)CCOCCNc4cccc5c4C(=O)N(C4CCC(=O)NC4=O)C5=O)c3)c2ncn1[C@H]1CC[C@H](O)CC1] |
| 30 | 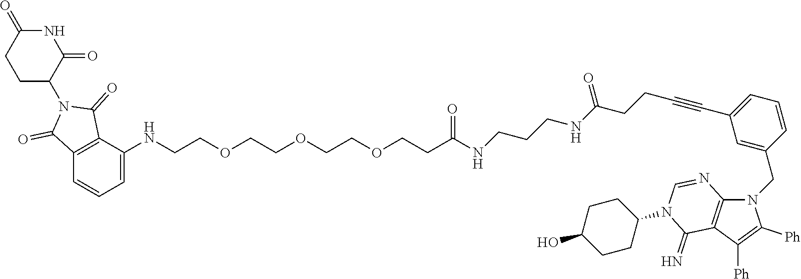 [SMILES: N=c1c2c(-c3ccccc3)c(-c3ccccc3)n(Cc3cccc(C#CCCC(=O)NCCCNC(=O)CCOCCOCCOCCNc4cccc5c4C(=O)N(C4CCC(=O)NC4=O)C5=O)c3)c2ncn1[C@H]1CC[C@H](O)CC1] |
| 31 | 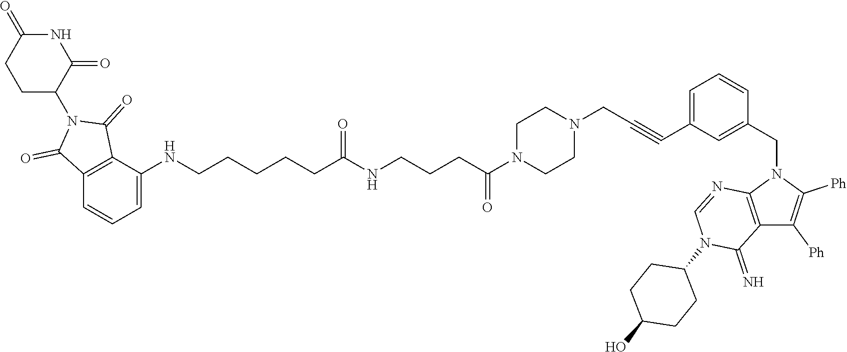 [SMILES: N=c1c2c(-c3ccccc3)c(-c3ccccc3)n(Cc3cccc(C#CCN4CCN(C(=O)CCCNC(=O)CCCCCNc5cccc6c5C(=O)N(C5CCC(=O)NC5=O)C6=O)CC4)c3)c2ncn1[C@H]1CC[C@H](O)CC1] |
| 32 | 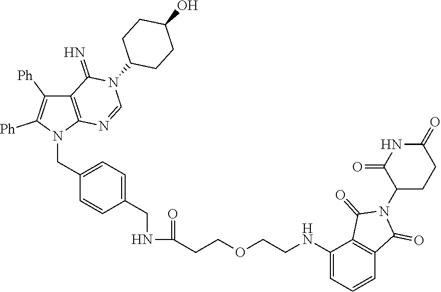 |
| 33 | 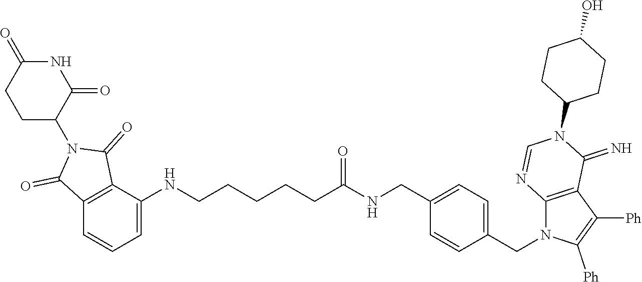 |
| 34 | 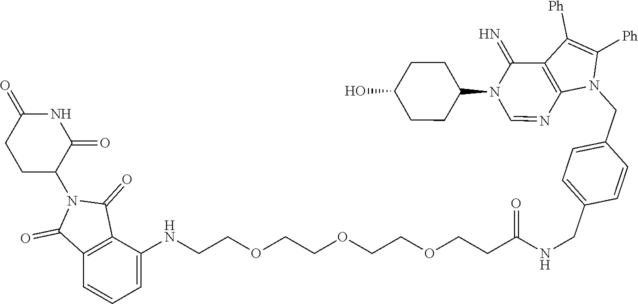 |
| 35 | 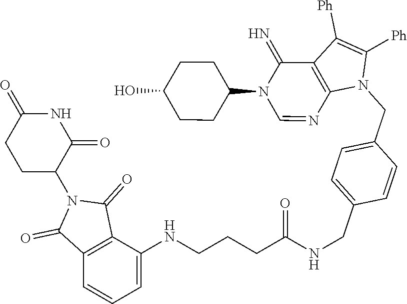 |
| 36 | 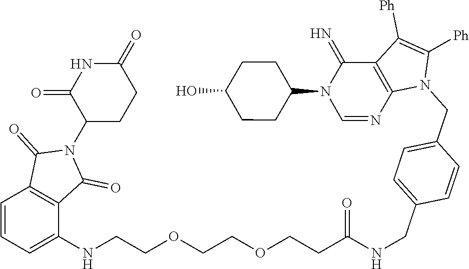 |
| 37 | 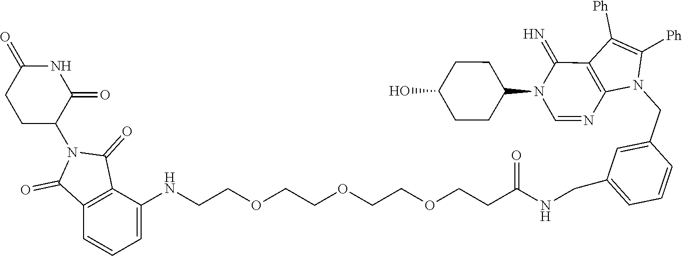 |
| 38 | 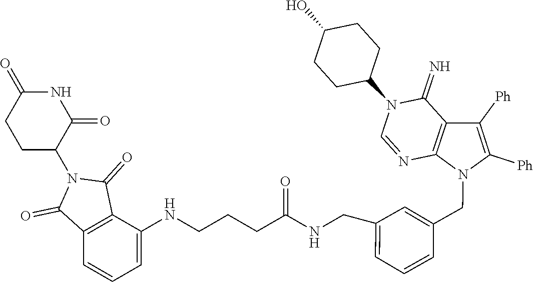 |
| 39 | 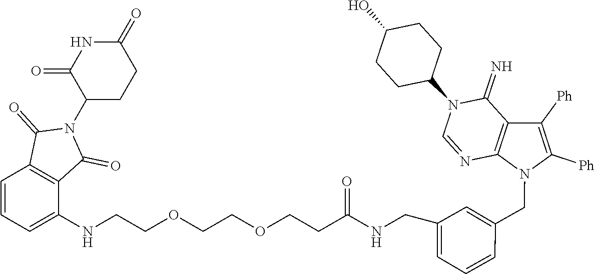 |
| 40 | 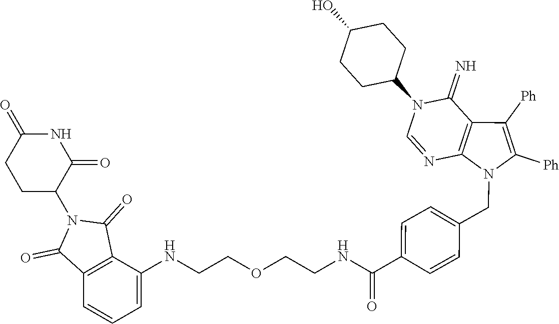 |
| 41 | 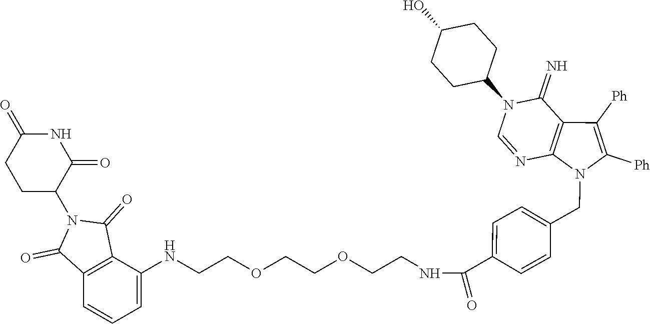 |
| 42 | 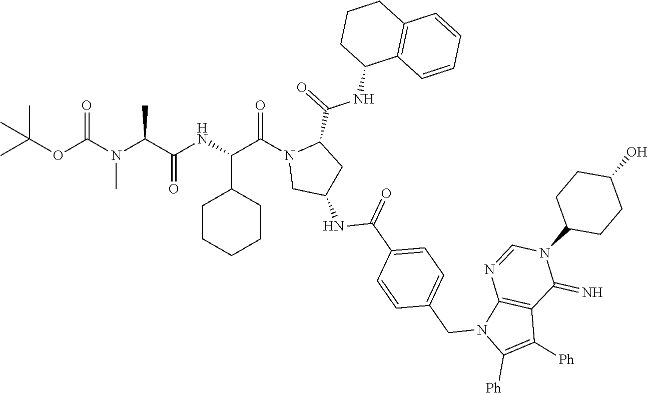 |
| 43 | 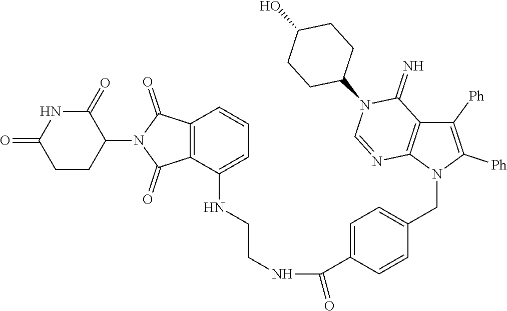 |
| 44 | 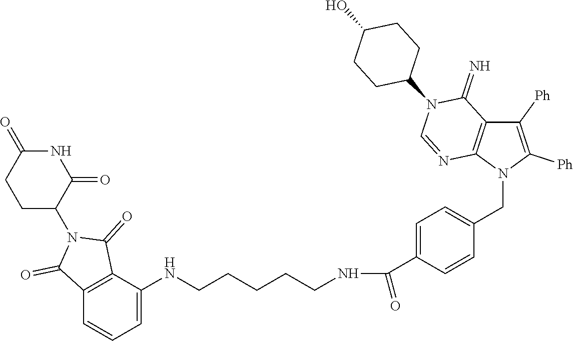 |
| 45 | 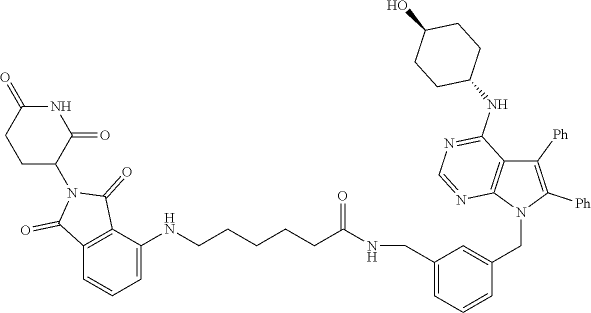 [SMILES: O=C(CCCCCNc1cccc2c1C(=O)N(C1CCC(=O)NC1=O)C2=O)NCc1cccc(Cn2c(-c3ccccc3)c(-c3ccccc3)c3c(N[C@H]4CC[C@H](O)CC4)ncnc32)c1] |
| 46 | 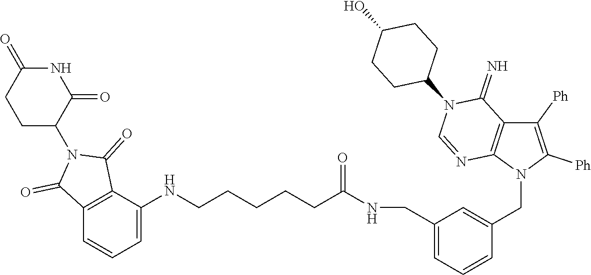 |
| 47 | 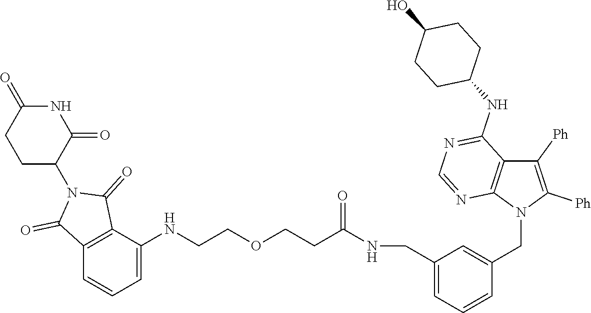 [SMILES: O=C(CCOCCNc1cccc2c1C(=O)N(C1CCC(=O)NC1=O)C2=O)NCc1cccc(Cn2c(-c3ccccc3)c(-c3ccccc3)c3c(N[C@H]4CC[C@H](O)CC4)ncnc32)c1] |
| 48 | 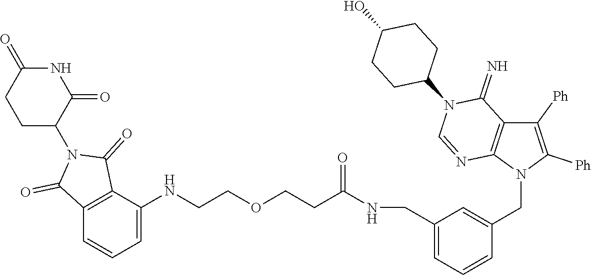 |
| 49 | 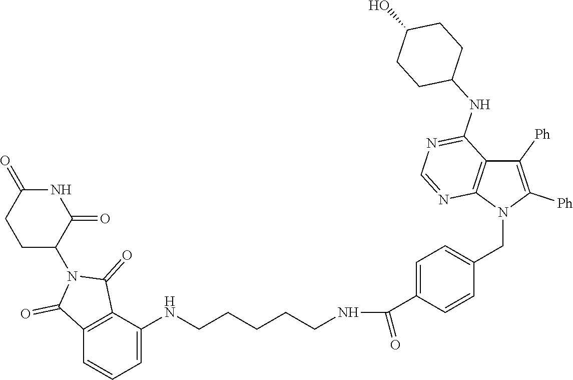 |
| 50 | 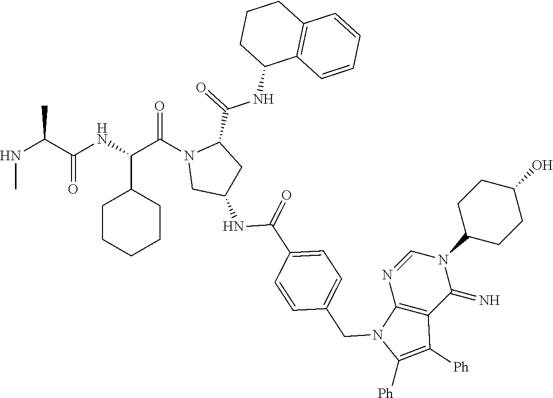 |
| 51 | 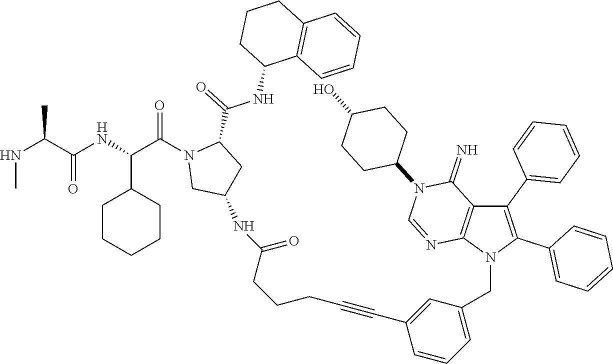 |
| 52 | 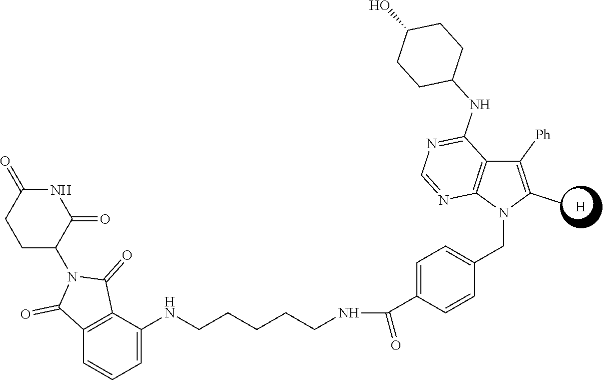 |
| 53 | 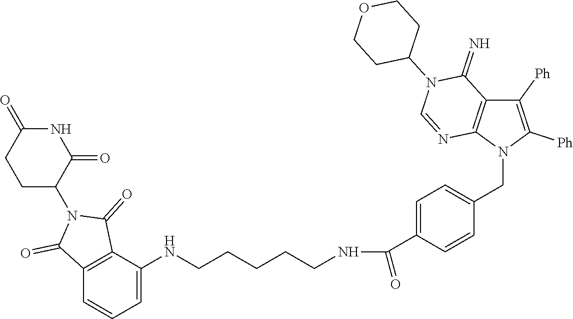 [SMILES: N=c1c2c(-c3ccccc3)c(-c3ccccc3)n(Cc3ccc(C(=O)NCCCCCNc4cccc5c4C(=O)N(C4CCC(=O)NC4=O)C5=O)cc3)c2ncn1C1CCOCC1] |
| 54 | 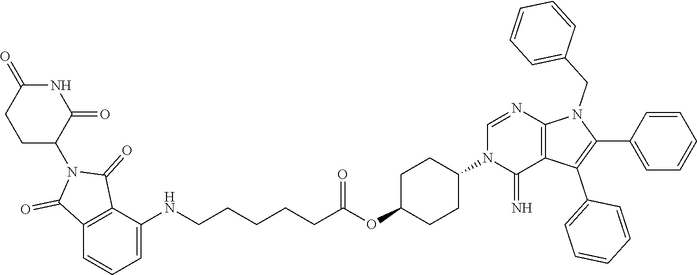 [SMILES: N=c1c2c(-c3ccccc3)c(-c3ccccc3)n(Cc3ccccc3)c2ncn1[C@H]1CC[C@H](OC(=O)CCCCCNc2cccc3c2C(=O)N(C2CCC(=O)NC2=O)C3=O)CC1] |
| 55 | 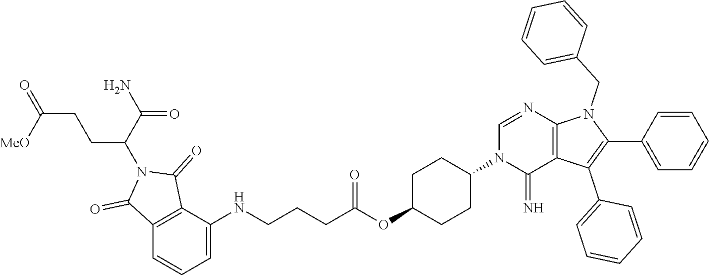 [SMILES: COC(=O)CCC(C(N)=O)N1C(=O)c2cccc(NCCCC(=O)O[C@H]3CC[C@H](n4cnc5c(c(-c6ccccc6)c(-c6ccccc6)n5Cc5ccccc5)c4=N)CC3)c2C1=O] |
| 56 | 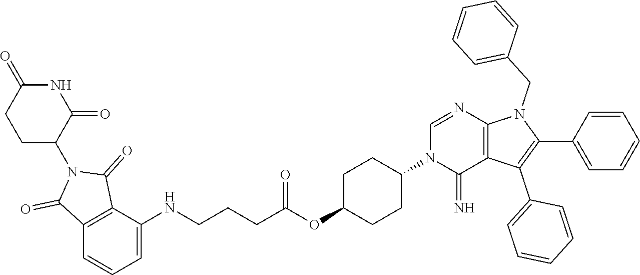 [SMILES: N=c1c2c(-c3ccccc3)c(-c3ccccc3)n(Cc3ccccc3)c2ncn1[C@H]1CC[C@H](OC(=O)CCCNc2cccc3c2C(=O)N(C2CCC(=O)NC2=O)C3=O)CC1] |
| 57 | 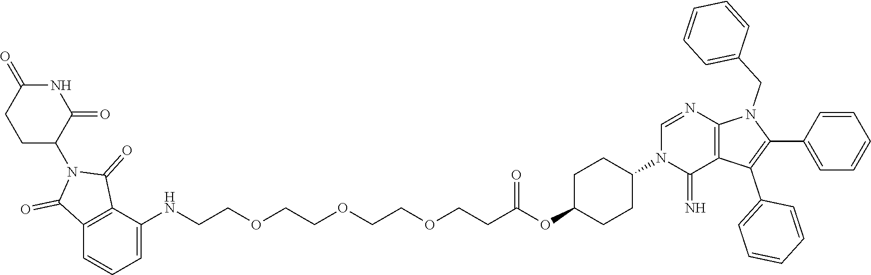 [SMILES: N=c1c2c(-c3ccccc3)c(-c3ccccc3)n(Cc3ccccc3)c2ncn1[C@H]1CC[C@H](OC(=O)CCOCCOCCOCCNc2cccc3c2C(=O)N(C2CCC(=O)NC2=O)C3=O)CC1] |

The compounds described herein may exist, in some cases, as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography and/or recrystallization or by the forming diastereomers and separation thereof (Jean Jacques, Andre Collet, Samuel H. Wilen, “Enantiomers, Racemates and Resolutions”, John Wiley And Sons, Inc., 1981). Stereoisomers may also be obtained by stereoselective synthesis using synthetic methods known in the art. In some embodiments, the compounds disclosed herein are enantiomers having an enantiomeric excess (% ee) of at least about 50%, about 60%, about 70%, about 80%, about 90%, about 95%, about 98%, or about 99.5%. In some embodiments, the compounds disclosed herein are diastereomers having a diastereomeric excess((% de) of atleast about 50%, about 60%, about 70%, about 80%, about 90%, about 95%, about 98%, or about 99.5%. In some embodiments, the compounds disclosed herein are present as enantiomeric or diastereomeric mixtures.

The methods and compositions described herein include the use of amorphous forms as well as crystalline forms (also known as polymorphs). The compounds described herein may be in the form of pharmaceutically acceptable salts. Active metabolites of these compounds having the same type of activity are included in the scope of the present disclosure.

In some embodiments, the compounds described herein may be formed as, and/or used as, pharmaceutically acceptable salts. The type of pharmaceutically acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

In some embodiments, the compounds and salts described herein include isotopically labeled compounds. In general, isotopically labeled compounds are identical to those recited in the various formulae and structures presented herein, but for the fact that one or more atoms are replaced by an atom having an atomic mass or mass number different from the atomic mass or mass number most common in nature. Examples of isotopes that can be incorporated into the present compounds include isotopes of hydrogen, carbon, nitrogen, oxygen, fluorine and chlorine, for example, 2H, 3H, 13C, 14C, 15N, 180, 170, 35S, 18F, 36Cl, respectively. Certain isotopically labeled compounds described herein, for example those into which radioactive isotopes such as 3H and 14C are incorporated, are useful in drug and/or substrate tissue distribution assays. Further, substitution with isotopes such as deuterium, i.e., 2H, can afford certain therapeutic advantages resulting from greater metabolic stability, such as, for example, increased in vivo half-life or reduced dosage requirements.

In some embodiments, the compounds disclosed herein bind to or inhibit eEF1A1 protein and exhibit an ranging from about 0.001 μM to about 1.0 μM, 0.001 μM to about 0.75 μM, from about 0.001 μM to about 0.5 μM, from about 0.01 μM to about 0.5 μM, or from about 0.01 μM to about 0.25 μM. In some embodiments, the values is less than about 1 μM, less than about 0.75 μM, less than about 0.50 μM, less than about 0.25 μM, or less than about 0.1 μM. A skilled artisan would be aware that ligand concentrations of the compounds disclosed herein and treatment time for maximal degradation as are widely known in the art to possess a “hook effect” where higher concentrations can lead to lower degradation of targeted proteins [33] and would optimize the ligand concentration accordingly.

In some embodiments, the compounds disclosed herein degrade eEF1A1 protein at concentrations ranging from about 0.001 μM to about 1.0 μM, 0.001 μM to about 0.75 μM, from about 0.001 μM to about 0.5 μM, from about 0.01 μM to about 0.5 μM, or from about 0.01 μM to about 0.25 μM. In some embodiments, the IC₅₀ values is less than about 1 μM, less than about 0.75 μM, less than about 0.50 μM, less than about 0.25 μM, or less than about 0.1 μM.

In some embodiments, the compound disclosed herein decreases the number of PNCs per cell (e.g., a cancerous cell) and exhibit and ranging from 0.001 μM to about 1.0 μM, 0.001 μM to about 0.75 μM, from about 0.001 μM to about 0.5 μM, from about 0.01 μM to about 0.5 μM, or from about 0.01 μM to about 0.25 μM. In some embodiments, the IC₅₀ values is less than about 1 μM, less than about 0.75 μM, less than about 0.50 μM, less than about 0.25 μM, or less than about 0.1 μM

In some embodiments, the compounds disclosed herein can be employed for at least for one or more uses of:
(1) Selective degradation of the target protein(s) of metarrestin that lead to PNC disruption. This includes eEF1A1 and eEF1A2 as well as previously undisclosed proteins that contribute to the effect of metarrestin on PNC prevalence; and/or
(2) Peripherally selective protein degradation (i.e. outside of the CNS); and/or
(3) Protein degradation leading to anti-metastasis effects; and/or
(4) Protein degradation leading to reduction in tumor volume.

### III. Pharmaceutical Compositions

In certain embodiments, compounds or salts of Formulae (A), (B), (I), (II), (III) and/or (IV) disclosed herein, are combined with one or more additional agents (e.g., pharmaceutically acceptable carriers) to form pharmaceutical compositions. Pharmaceutical compositions may be formulated in a conventional manner using one or more physiologically acceptable carriers including excipients and auxiliaries, which facilitate processing of the active compounds into preparations which can be used pharmaceutically. Proper formulation is dependent upon the route of administration chosen. Additional details about suitable excipients for pharmaceutical compositions described herein may be found, for example, in Remington: The Science and Practice of Pharmacy, Nineteenth Ed. (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of a compound or salt of Formulae (I), (II) and/or (III) with any suitable substituents and functional groups disclosed herein, with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. The compounds or salts of Formulae (I), (II) and/or (III) with any suitable substituents and functional groups disclosed herein can be used singly or in combination with one or more therapeutic agents as components of mixtures (as in combination therapy).

The pharmaceutical formulations described herein can be administered to a subject by multiple administration routes, including but not limited to, oral, parenteral (e.g., intravenous, subcutaneous, intramuscular), intranasal, buccal, nasal, pulmonary, topical, rectal, vaginal, intratumoral, or transdermal administration routes. Moreover, the pharmaceutical compositions described herein, which include a compound of Formulae (I), (II) and/or (III) with any suitable substituents and functional groups disclosed herein, can be formulated into any suitable dosage form, including but not limited to, aqueous oral dispersions, liquids, gels, syrups, elixirs, slurries, suspensions, aerosols, fast melt formulations, effervescent formulations, lyophilized formulations, tablets, powders, pills, dragées, and capsules.

One may administer the compounds and/or compositions in a local rather than systemic manner, for example, via injection of the compound directly into an organ or tissue, often in a depot preparation or sustained release formulation. Such long-acting formulations may be administered by implantation (for example subcutaneously or intramuscularly) orby intramuscular injection. Furthermore, one may administer the drug in a targeted drug delivery system, for example, in a liposome coated with organ-specific antibody. The liposomes will be targeted to and taken up selectively by the organ. In addition, the drug may be provided in the form of a rapid release formulation, in the form of an extended release formulation, or in the form of an intermediate release formulation.

Pharmaceutical compositions including a compound described herein may be manufactured in a conventional manner, such as, by way of example only, by means of conventional mixing, dissolving, granulating, dragée-making, levigating, emulsifying encapsulating, entrapping or compression processes.

The pharmaceutical compositions will include at least one compound of Formulae (A), (B), (I), (II), (III) and/or (IV) as disclosed herein, as an active ingredient in free-acid or free-base form, or in a pharmaceutically acceptable salt form.

In some embodiments, compositions provided herein may also include one or more preservatives to inhibit microbial activity. Suitable preservatives include quaternary ammonium compounds such as benzalkonium chloride, cetyltrimethylammonium bromide and cetylpyridinium chloride.

In some embodiments, the pharmaceutical solid dosage forms described herein can include a compound of Formulae (A), (B), (I), (II), (III) and/or (IV) and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the formulation of the compound described herein. In one embodiment, some or all of the particles of the compound described herein are coated. In another embodiment, some or all of the particles of the compound described herein are microencapsulated. In still another embodiment, the particles of the compound described herein are not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose (HPMC), hydroxypropylmethylcellulose acetate stearate (HPMCAS), sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, HPMC, hydroxypropylmethycellulose phthalate, HPMCAS, sucrose, xylitol, lactitol, mannitol, sorbitol, sodium chloride, polyethylene glycol, and the like.

Suitable disintegrants for use in the solid dosage forms described herein include, but are not limited to, natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH 101, Avicel® PH 102, Avicel® PH 105, Elcema® P100, Emcocel®, Vivacell®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Suitable binders in the solid dosage forms described herein include, but are not limited to, carboxymethylcellulose, methylcellulose (e.g., Methocel®), hydroxypropylmethylcellulose (e.g, Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g, Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Di-Pac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabinogalactan, Veegum®, polyethylene glycol, waxes, sodium alginate, and the like.

Suitable lubricants or glidants for use in the solid dosage forms described herein include, but are not limited to, stearic acid, calcium hydroxide, talc, corn starch, sodium stearyl fumarate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10@), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 5400 to about 7000, vinyl pyrrolidone/vinyl acetate copolymer (S630), sodium carboxymethylcellulose, methylcellulose, hydroxypropylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

There is considerable overlap between additives used in the solid dosage forms described herein. Thus, the above-listed additives should be taken as merely exemplary, and not limiting of the types of additives that can be included in solid dosage forms of the pharmaceutical compositions described herein.

Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002).

The pharmaceutical compositions described herein may include sweetening agents such as, but not limited to, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glycyrrhizinate (MagnaSweet®), maltol, mannitol, maple, marshmallow, menthol, mint cream, mixed berry, neohesperidin DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

Potential excipients for intranasal formulations include formulations solutions in saline, employing benzyl alcohol or other suitable preservatives, fluorocarbons, and/or other solubilizing or dispersing agents. See, e.g., Ansel, H. C. et al., Pharmaceutical Dosage Forms and Drug Delivery Systems, Sixth Ed. (1995). Preferably, these compositions and formulations are prepared with suitable nontoxic pharmaceutically acceptable ingredients. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. Preferably, the nasal dosage form should be isotonic with nasal secretions.

For administration by inhalation, the compounds described herein may be in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

Buccal formulations that include compounds described herein may be administered using a variety of formulations, which include, but are not limited to, U.S. Pat. Nos. 4,229,447, 4,596,795, 4,755,386, and 5,739,136. In addition, the buccal dosage forms described herein can further include a bioerodible (hydrolysable) polymeric carrier that also serves to adhere the dosage form to the buccal mucosa. The buccal dosage form is fabricated to erode gradually over a predetermined time period, wherein the delivery of the compound is provided essentially throughout. Buccal drug delivery avoids the disadvantages encountered with oral drug administration, e.g., slow absorption, degradation of the active agent by fluids present in the gastrointestinal tract and/or first-pass inactivation in the liver. With regard to the bioerodible (hydrolysable)polymeric carrier, virtually any such carrier can be used, so long as the desired drug release profile is not compromised, and the carrier is compatible with the compounds described herein, and any other components that may be present in the buccal dosage unit. Generally, the polymeric carrier comprises hydrophilic (water-soluble and water-swellable) polymers that adhere to the wet surface of the buccal mucosa. Examples of polymeric carriers useful herein include acrylic acid polymers and copolymers, e.g., those known as “carbomers” (Carbopol®, which may be obtained from B.F. Goodrich, is one such polymer). Other components may also be incorporated into the buccal dosage forms described herein include, but are not limited to, disintegrants, diluents, binders, lubricants, flavoring, colorants, preservatives, and the like. For buccal or sublingual administration, the compositions may take the form of tablets, lozenges, or gels formulated in a conventional manner.

Transdermal formulations described herein may incorporate certain pharmaceutically acceptable excipients, which are conventional in the art. In some embodiments, formulations suitable for transdermal administration of compounds described herein may employ transdermal delivery devices and transdermal delivery patches and can be lipophilic emulsions or buffered, aqueous solutions, dissolved and/or dispersed in a polymer or an adhesive.

Formulations suitable for intramuscular, subcutaneous, or intravenous injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Formulations suitable for subcutaneous injection may also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections, compounds described herein may be formulated in aqueous solutions, preferably in physiologically compatible buffers such as Hanks solution, Ringer's solution, or physiological saline buffer. For transmucosal administration, penetrants appropriate to the barrier to be permeated are used in the formulation. Such penetrants are generally recognized in the field. For other parenteral injections, appropriate formulations may include aqueous or nonaqueous solutions, preferably with physiologically compatible buffers or excipients. Such excipients are generally recognized in the field.

Parenteral injections may involve bolus injection or continuous infusion. Formulations for injection may be presented in unit dosage form, e.g., in ampoules or in multi-dose containers, with an added preservative. The pharmaceutical composition described herein maybe in a form suitable for parenteral injection as a sterile suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethylcellulose, sorbitol, or dextran. Optionally, the suspension may also contain suitable stabilizers or agents, which increase the solubility of the compounds to allow for the preparation of highly concentrated solutions. Alternatively, the active ingredient may be in powder form for constitution with a suitable vehicle, e.g., sterile pyrogen-free water, before use.

In certain embodiments, delivery systems for pharmaceutical compounds may be employed, such as, for example, liposomes and emulsions. In certain embodiments, compositions provided herein also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein may be administered topically and are formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

The compounds described herein may also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In some embodiments, the compounds of Formulae (A), (B), (I), (II), (III) and/or (IV) disclosed herein are combined with other therapeutic agents, such as other anti-cancer agents, anti-allergic agents, anti-nausea agents (or anti-emetics), pain relievers, cytoprotective agents, and combinations thereof.

In another embodiment, the compounds of Formulae (A), (B), (I), (II), (III) and/or (IV) disclosed herein are combined with reversible DNA binders, DNA alkylators, DNA strand breakers, or any combination thereof.

Examples of suitable reversible DNA binders include topetecan hydrochloride, irinotecan (CPT11-Camptosar), rubitecan, exatecan, nalidixic acid, TAS-103, etoposide, acridines (such as amsacrine, aminacrine), actinomycins (such as actinomycin D), anthracyclines (such as doxorubicin, daunorubicin), benzophenainse, XR 11576/MLN 576, benzopyridoindoles, Mitoxantrone, AQ4, Etoposide, Teniposide, (epipodophyllotoxins), and bisintercalating agents such as triostin A and echinomycin.

Examples of suitable DNA alkylators include sulfur mustard, the nitrogen mustards (such as mechlorethamine), chlorambucil, melphalan, ethyleneimines (such as triethylenemelamine, carboquone, diaziquone), methyl methanesulfonate, busulfan, CC-1065, duocaimycins (such as duocarmycin A, duocarmycin SA), metabolically activated alkylating agents such as nitrosoureas (such as carmustine, lomustine, (2-chloroethyl)nitrosoureas), triazine antitumor drugs such as triazenoimidazole (such as dacarbazine), mitomycin C, leinamycin, and the like.

Examples of suitable DNA strand breakers include doxorubicin and daunorubicin (which are also reversible DNA binders), other anthracyclines, bleomycins, tirapazamine, enediyne antitumor antibiotics such as neocarzinostatin, esperamicins, calicheamicins, dynemicin A, hedarcidin, C-1027, N1999A2, esperamicins, zinostatin, and the like.

Generally, an agent, such as a compound of Formulae (A), (B), (I), (II), (III) and/or (IV) disclosed herein, is administered in an amount effective for treating the disease or disorder (i.e., a therapeutically effective amount). Thus, a therapeutically effective amount can be an amount that is capable of at least partially treating, preventing or reversing a disease or disorder. The dose required to obtain an effective amount may vary depending on the agent, formulation, disease or disorder, and individual to whom the agent is administered.

Determination of effective amounts may also involve in vitro assays in which varying doses of agent are administered to cells (e.g., cancerous cells) in culture and the concentration of agent effective for ameliorating some or all symptoms is determined in order to calculate the concentration required in vivo. Effective amounts may also be based on in vivo animal studies.

An agent can be administered prior to, concurrently with, and subsequent to the appearance of symptoms of a disease or disorder. In some embodiments, an agent is administered to a subject with a family history of the disease or disorder, or who has a phenotype that may indicate a predisposition to a disease or disorder, or who has a genotype which predisposes the subject to the disease or disorder.

In some embodiments, the compositions described herein are provided as pharmaceutical and/or therapeutic compositions. The pharmaceutical and/or therapeutic compositions of the present disclosure can be administered in a number of ways depending upon whether local or systemic treatment is desired and upon the area to be treated. Administration can be topical (including ophthalmic and to mucous membranes including vaginal and rectal delivery), pulmonary (e.g., by inhalation or insufflation of powders or aerosols, including by nebulizer, intratracheal, intranasal, epidermal and transdermal), oral or parenteral. Parenteral administration includes intravenous, intra-arterial, subcutaneous, intraperitoneal or intramuscular injection or infusion; or intracranial, e.g., intrathecal or intraventricular, administration. Compositions and formulations for topical administration can include transdermal patches, ointments, lotions, creams, gels, drops, suppositories, sprays, liquids and powders. Conventional carriers; aqueous, powder, or oily bases; thickeners; and the like can be necessary or desirable. Compositions and formulations for oral administration include powders or granules, suspensions or solutions in water or non-aqueous media, capsules, sachets or tablets. Thickeners, flavoring agents, diluents, emulsifiers, dispersing aids or binders can be desirable. Compositions and formulations for parenteral, intrathecal or intraventricular administration can include sterile aqueous solutions that can also contain buffers, diluents and other suitable additives such as, but not limited to, penetration enhancers, carrier compounds and other pharmaceutically acceptable carriers or excipients. Pharmaceutical and/or therapeutic compositions of the present disclosure include, but are not limited to, solutions, emulsions, and liposome-containing formulations. These compositions can be generated from a variety of components that include, but are not limited to, preformed liquids, self-emulsifying solids and self-emulsifying semisolids.

The pharmaceutical and/or therapeutic formulations, which can conveniently be presented in unit dosage form, can be prepared according to conventional techniques well known in the pharmaceutical/nutriceutical industries. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product. The compositions of the present disclosure can be formulated into any of many possible dosage forms such as, but not limited to, tablets, capsules, liquid syrups, soft gels, suppositories, and enemas. The compositions of the present disclosure can also be formulated as suspensions in aqueous, non-aqueous, oil-based, or mixed media. Suspensions can further contain substances that increase the viscosity of the suspension including, for example, sodium carboxymethylcellulose, sorbitol and/or dextran. The suspension can also contain stabilizers. In one embodiment of the present disclosure, the pharmaceutical compositions can be formulated and used as foams. Pharmaceutical foams include formulations such as, but not limited to, emulsions, microemulsions, creams, jellies and liposomes. While similar in nature these formulations vary in the components and the consistency of the final product.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

Dosing and administration regimes are tailored by the clinician, or others skilled in the pharmacological arts, based upon well-known pharmacological and therapeutic considerations including, but not limited to, the desired level of therapeutic effect, and the practical level of therapeutic effect obtainable. Generally, it is advisable to follow well-known pharmacological principles for administrating chemotherapeutic agents (e.g., it is generally advisable to not change dosages by more than 50% at time and no more than every 3-4 agent half-lives). For compositions that have relatively little or no dose-related toxicity considerations, and where maximum efficacy is desired, doses in excess of the average required dose are not uncommon. This approach to dosing is commonly referred to as the “maximal dose” strategy. In certain embodiments, the compounds are administered to a subject at a dose of about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g, as sensitizing agents), the effective amount may be less than when the agent is used alone. Dosing may be once per day or multiple times per day for one or more consecutive days.

### IV. Methods of Treatment

The present disclosure provides compounds and methods for binding to and/or inhibiting the activity of metarrestin targets. Exemplary metarrestin targets include, but should not be limited to, the translation elongation factor eEF1A2 protein, RNA polymerases (e.g., I, II, and/or III), and/or the perinucleolar compartment (PNC). In some embodiments, the present disclosure provides compounds and methods that modulate the activity of metarrestin targets by binding to and/or inhibiting the target directly. In some embodiments, the compounds provided in the present disclosure modulate the activity of metarrestin targets indirectly, for example, by binding to and/or inhibiting cellular upstream/downstream targets associated with the metarrestin targets. In some embodiments such metarrestin targets are responsible for modulating the PNC activity of the cell. As such, modulating the activity of metarrestin targets directly or indirectly can result in a reductions of PNC activity.

Inhibition of metarrestin targets(s) may be assessed and demonstrated by a wide variety of ways known in the art. Non-limiting examples include measuring (a) a direct decrease in metarrestin target activity (e.g., translation elongation factor eEF1A2 protein activity, RNA polymerase transcription activity, number of PNCs/cell); (b) a directed decrease in the concentration/amount and/or protein level of a metarrestin target; (c) a decrease in cell proliferation and/or cell viability; (d) an increase in cell differentiation; (e) a decrease in cell proliferation spreading across multiple tissue types; (f) a decrease in the levels of downstream targets of metarrestin target activity; and (g) decrease in tumor volume and/or tumor volume growth rate. It should be noted that exemplary cells include normal cells as well as cancerous cells. Kits and commercially available assays can be utilized for determining one or more of the above.

Binding of compounds disclosed herein to the eEF1A protein can be determined using known methods in the arts, such as differential scanning fluorimetry (DSF) or cellular thermal shift assay (CETSA).

PNC inhibitory activity was determined using fluorescent imaging techniques known in the art, such as: a CellTiter-Glo® luminescence assay from Promega to determine cell viability and/or fluorescent imaging studies with an IN Cell Analyzer 1000 automated system in combination with a green fluorescent protein (GFP) tagged PNC localized complex.

The disclosure provides compounds and methods for treating a subject suffering from a disease, comprising administering a compound or salt described herein, for example, a compound, or salt of Formulae (I), (II) and/or (III) disclosed herein, to the subject. In some embodiments, the disease is selected from a disease associated with a metarrestin target (e.g., eEF1A protein) expression (e.g., aberrant expression, overexpression, etc.), and/or concentration (e.g., PNC), and/or activity (e.g., cancer). In certain embodiments, the disease is mediated by the metarrestin target (e.g., eEF1A2 protein) activity, and/or concentration (e.g., PNC) and/or expression (e.g., aberrant expression, overexpression, etc.). In some embodiments, the disease or condition is treatable by inhibition of and/or degradation of the metarrestin target (e.g., translation elongation factor eEF1A2 protein). In some embodiments, the method comprises treating a disease or condition that is treatable by inhibition of the metarrestin target (e.g., eEF1A2 protein, RNA polymerases I, II, III, and/or PNC) by administering to a subject in need thereof a therapeutically effective amount of a compound or a salt thereof of Formulae (A), (B), (I), (II), (III) and/or (IV) or a pharmaceutical composition as disclosed herein.

In some embodiments, the disclosure provides a method for treating cancer in a subject, comprising administering a compound or salt described herein, for example, a compound or salt of Formulae (A), (B), (I), (II), (III) and/or (IV) disclosed herein, to the subject. In some embodiments, the cancer is mediated by a metarrestin target (e.g., aberrant expression, overexpression, concentration etc.) and/or activity. In some embodiments, the cancer is a metastatic cancer.

In certain embodiments, the disclosure provides a method of treating a disease in a subject, wherein the method comprises determining if the subject has a metarrestin target-mediated condition (e.g., cancer) and administering to the subject a therapeutically effective dose of a compound or salt described herein, for example, a compound or salt of Formulae (I), (II), and/or (III) as disclosed herein. In some embodiments, the metarrestin target can be any cellular target that is able to modulate the PNC activity (i.e., PNC reducing activity) of a cancer cell. In some embodiments, the metarrestin target is selected from the group consisting of eEF1A, RNA polymerase I. II. III, and/or PNC.

Determining whether a tumor or cancer expresses (e.g., overexpresses, aberrantly expresses, etc.) eEF1A2 protein can be under taken by assessing the nucleotide sequence encoding eEF1A2 or by assessing the amino acid sequence of eEF1A2. Methods for detecting an eEF1A2 nucleotide sequence are known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. Methods for detecting an eEF1A2 protein are known by those of skill in the art. These methods include, but are not limited to, detection using a binding agent, e.g., an antibody, specific for eEF1A2, protein electrophoresis and Western blotting, and direct peptide sequencing.

Methods for determining whether a tumor or cancer expresses (e.g., overexpresses, aberrantly expresses, etc.) eEF1A2 protein or is mediated by eEF1A2 activity can use a variety of samples. In some embodiments, the sample is taken from a subject having a tumor or cancer. In some embodiments, the sample is taken from a subject having a cancer or tumor. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is processed to a cell lysate. In some embodiments, the sample is processed to DNA or RNA.

In certain embodiments, the disclosure provides a method of inhibiting eEF1A2 activity in a sample, comprising administering the compound or salt described herein to said sample comprising eEF1A2.

The disclosure provides methods for treating a disease by administering a compound or salt of Formulae (A), (B), (I), (II), (III), and/or (IV) or salt thereof, to a subject suffering from the disease, wherein the compound binds to the metarrestin target (e.g., eEF1A2 protein) and/or inhibits the metarrestin target (e.g., eEF1A2 protein, RNA polymerase I, II, III and/or PNC) activity. In some embodiments, the compound covalently binds to the metarrestin target (e.g., eEF1A2 protein). In some embodiments, the compound noncovalently binds to the metarrestin target (e.g., eEF1A2 protein). In some embodiments, the compound degrades the metarrestin target (e.g., eEF1A2 protein).

The disclosure also relates to a method of treating a hyperproliferative disorder in a mammal that comprises administering to the mammal a therapeutically effective amount of a compound or salt of Formulae (A), (B), (I), (II), (III), and/or (IV) or salt thereof with any suitable substituents and functional groups disclosed herein. In some embodiments, the method relates to the treatment of cancer, particularly metastatic cancer, such as adrenocortical carcinoma, AIDS-related lymphoma, AIDS-related malignancies, anal cancer, cerebellar astrocytoma, extrahepatic bile duct cancer, bladder cancer, osteosarcoma/malignant fibrous histiocytoma, brain stem glioma, ependymoma, visual pathway and hypothalamic gliomas, breast cancer, bronchial adenomas/carcinoids, carcinoid tumors, gastrointestinal carcinoid tumors, carcinoma, adrenocortical, islet cell carcinoma, primary central nervous system lymphoma, cerebellar astrocytoma, cervical cancer, chronic lymphocytic leukemia, chronic myelogenous leukemia, clear cell sarcoma of tendon sheaths, colon cancer, colorectal cancer, cutaneous t-cell lymphoma, endometrial cancer, ependymoma, esophageal cancer, Ewing's sarcoma/family of tumors, extracranial germ cell tumors, extragonadal germ cell tumors, extrahepatic bile duct cancer, eye cancers, including intraocular melanoma, and retinoblastoma, gallbladder cancer, gastrointestinal carcinoid tumor, ovarian germ cell tumor, gestational trophoblastic tumor, hairy cell leukemia, head and neck cancer, Hodgkin's disease, hypopharyngeal cancer, hypothalamic and visual pathway glioma, intraocular melanoma, Kaposi's sarcoma, laryngeal cancer, acute lymphoblastic leukemia, acute myeloid leukemia, liver cancer, non-small cell lung cancer, small cell lung cancer, non-Hodgkin's lymphoma, Waldenstrom's macroglobulinemia, malignant mesothelioma, malignant thymoma, medulloblastoma, melanoma, intraocular melanoma, merkel cell carcinoma, metastatic squamous neck cancer with occult primary, multiple endocrine neoplasia syndrome, multiple myeloma/plasma cell neoplasm, mycosis fungoides, myelodysplastic syndrome, chronic myelogenous leukemia, myeloid leukemia, multiple myeloma, myeloproliferative disorders, nasal cavity and paranasal sinus cancer, nasopharyngeal cancer, neuroblastoma, oral cancer, oral cavity and lip cancer, oropharyngeal cancer, osteosarcoma/malignant fibrous histiocytoma of bone, ovarian cancer, ovarian low malignant potential tumor, pancreatic cancer, paranasal sinus and nasal cavity cancer, parathyroid cancer, penile cancer, pheochromocytoma, pituitary tumor, pleuropulmonary blastoma, prostate cancer, rectal cancer, renal cell (kidney) cancer, transitional cell cancer (such as renal pelvis and ureter), retinoblastoma, rhabdomyosarcoma, salivary gland cancer, malignant fibrous histiocytoma of bone, soft tissue sarcoma, sezary syndrome, skin cancer, small intestine cancer, stomach (gastric) cancer, supratentorial primitive neuroectodermal and pineal tumors, cutaneous T-cell lymphoma, testicular cancer, malignant thymoma, thyroid cancer, gestational trophoblastic tumor, urethral cancer, uterine sarcoma, vaginal cancer, vulvar cancer, and Wilms' tumor.

In some embodiments, the cancer can be any cancer in any organ, for example, a cancer is selected from the group consisting of brain carcinoma, glioma, thyroid carcinoma, breast carcinoma, small-cell lung carcinoma, non-small-cell carcinoma, gastric carcinoma, colon carcinoma, gastrointestinal stromal carcinoma, pancreatic carcinoma, bile duct carcinoma, CNS carcinoma, ovarian carcinoma, endometrial carcinoma, prostate carcinoma, renal carcinoma, anaplastic large-cell lymphoma, leukemia, multiple myeloma, mesothelioma, and melanoma, and combinations thereof.

Subjects that can be treated with compounds of Formulae (I), (II) and/or (III) or salt thereof, or pharmaceutically acceptable salt, stereoisomer, diastereomer, or enantiomer of the compounds, according to the methods of this disclosure include, for example, subjects that have been diagnosed as having acute myeloid leukemia, acute myeloid leukemia, cancer in adolescents, adrenocortical carcinoma childhood, AIDS-related cancers, e.g., Lymphoma and Kaposi's Sarcoma, anal cancer, appendix cancer, astrocytomas, atypical teratoid, basal cell carcinoma, bile duct cancer, bladder cancer, bone cancer, brain stem glioma, brain tumor, breast cancer, bronchial tumors, burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myeloproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, hodgkin lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, Viral-Induced cancer, leukemia, hematologic malignancy, solid tumor cancer, prostate cancer, castration-resistant prostate cancer, breast cancer, Ewing's sarcoma, bone sarcoma, primary bone sarcoma, T-cell prolymphocyte leukemia, glioma, glioblastoma, hepatocellular carcinoma, liver cancer, or diabetes. In some embodiments subjects that are treated with the compounds of the disclosure include subjects that have been diagnosed as having a metastatic cancer selected from breast cancer, prostate cancer, lung cancer, kidney cancer, thyroid cancer, colon cancer, pancreatic cancer, bone cancer, and liver cancer.

The disclosure further provides methods of reducing metastasis in a subject diagnosed with a metastatic cancer, the method comprising administering an effective amount of a compound of salt of Formulae (A), (B), (I), (II), (III) and/or (IV) or salt thereof to a subject, wherein metastasis is reduced by atleast about 10%, about 20%, about 30%, about 40%, about 50%, about 60%, about 70%, about 80% or atleast about 90% compared to a subject that is not treated with the compounds disclosed herein.

In some embodiments, the disclosure provides a method for reducing the colony formation of cancer cells in a mammal comprising administering to a mammal in need thereof a compound of Formulae (A), (B), (I), (II), (III) and/or (IV) or a salt thereof. In some embodiments, colony formation is reduced by atleast about 10%, about 20%, about 30%, about 40%, about 50%, about 60%, about 70%, about 80% or at least about 90% compared to a mammal that is not treated with the compounds disclosed herein.

In some embodiments, the disclosure provides a method of reducing the migration of cancer cells in a mammal comprising administering to a mammal in need thereof a compound of Formulae (A), (B), (I), (II), (III) and/or (IV) or a salt thereof. In some embodiments, the migration of cancer cells is reduced by at least about 10%, about 20%, about 30%, about 40%, about 50%, about 60%, about 70%, about 80% or at least about 90% compared to a mammal that is not treated with the compounds disclosed herein.

The disclosure further provides methods of inhibiting eEF1A2 protein activity, by contacting the eEF1A2 protein with an effective amount of a compound or salt of Formulae (I), (II) and/or (III) or salt thereof (e.g., by contacting a cancer cell, tissue, or organ that expresses the eEF1A2 protein). In someembodiments, thedisclosureprovidesmethodsofinhibitingtheeEF1A2 protein activity in a subject including but not limited to rodents and mammals, e.g., humans, by administering to the subject an effective amount of a compound of Formulae (A), (B), (I), (II) (III) and/or (IV) or salt thereof. Thus, the present disclosure is directed to methods of inhibiting the eEF1A2 protein activity in an in vitro and in vivo testing environment, which a skilled artisan would be familiar with. In some embodiments, the percentage of inhibition of the eEF1A2 protein in vitro and/or in vivo is at least 25%, 30%, 40%, 50%, 60%, 70%, 80%, or 90%.

For example, in some embodiments, the disclosure provides methods of inhibiting the eEF1A2 protein activity in a cancer cell by contacting the cancer cell with an amount of a compound as disclosed herein sufficient to inhibit the activity. In some embodiments, the disclosure provides methods of inhibiting the eEF1A2 protein activity in a tissue (e.g., cancerous tissue) by contacting the tissue with an amount of a compound or salt of Formulae (I), (II) and/or (III) or salt thereof, sufficient to inhibit the eEF1A2 protein activity in the tissue. In some embodiments, the disclosure provides methods of inhibiting the eEF1A2 protein activity in an organism (e.g., mammal, human, etc.) by contacting the organism with an amount of a compound or salt of Formulae (A), (B), (I), (II), (III) and/or (IV) or salt thereof, sufficient to inhibit the eEF1A2 protein activity in the organism.

In some embodiments, the methods disclosed herein are directed to compounds that are able to degrade metarrestin targets, e.g., the eEF1A2 protein. For example, methods of degrading 10 metarrestin targets (e.g., the eEF1A2 protein) comprises contacting the metarrestin target (i.e., the eEF1A2 protein) with an effective amount of a compound or salt of Formulae (I), (II) and/or (III) or salt thereof (e.g., by contacting a cancer cell, tissue, or organ that expresses metarrestin targets, e.g., the eEF1A2 protein). In some embodiments, the disclosure provides methods of degrading the metarrestin targets, e.g., eEF1A2 protein, in a subject including, but not limited to, rodents and mammals, e.g., humans, by administering to the subject an effective amount of a compound of Formulae (A), (B), (I), (II), (III) and/or (IV) or salt thereof. In some embodiments, the percentage of degradation of the eEF1A2 protein is at least 25%, 30%, 40%, 50%, 60%, 70%, 80%, or 90%. In some embodiments, the metarrestin targets include, but are not limited to, the eEF1A2 protein, RNA polymerases (e.g., I, II, III), and/or perinuclear compartment (PNC) and/or proteins modulating the PNC.

In some embodiments, methods of inhibiting eEF1A2 protein activity results in a decrease in RNA polymerase activity as eEF1A protein interacts with RNA polymerase (i.e., I, II, and/or III) during transcription, when an effective amount of a compound or salt of Formulae (I), (II) and/or (III) or salt thereof disclosed herein is contacted with a cancer cell, tissue, or organ that expresses the eEF1A2 protein. In some embodiments, the disclosure provides methods of inhibiting RNA polymerase activity, e.g., transcription activity, in a subject including but not limited to rodents and mammals, e.g., humans, by administering to the subject an effective amount of a compound of Formulae (A), (B), (I), (II), (III) and/or (IV) or salt thereof. Thus, the present disclosure is directed to methods of inhibiting the RNA polymerase activity, e.g., RNA polymerase transcription, in an in vitro and in vivo testing environment, which a skilled artisan would be familiar with. In some embodiments, the percentage of inhibition of the RNA polymerase activity in vitro and/or in vivo is at least 25%, 30%, 40%, 50%, 60%, 70%, 80%, or 90%.

In some embodiments, the methods disclosed herein are directed to a decrease in the number of PNCs per cell (e.g., cancer cell), the method comprising contacting a cell, tissue, or organ with an effective amount of a compound of Formulae (A), (B), (I), (II), (III) and/or (IV) or salt thereof. In some embodiments, such cell, tissue, or organ can be cancerous. In some embodiments, the disclosure provides methods of reducing the number of PNCs per cell, in a subject including but not limited to rodents and mammals, e.g., humans, by administering to the subject an effective amount of a compound of Formulae (A), (B), (I), (II), (III) and/or (IV) or salt thereof. Thus, the present disclosure is directed to methods of decreasing the number of PNCs per cell (e.g., cancer cell), in an in vitro and in vivo testing environment, which a skilled artisan would be familiar with. In some embodiments, the percentage of reduction in the number of PNCs per cell (e.g., cancer cell) in vitro and/or in vivo being at least about 10%, about 25%, about 30%, about 40%, about 50%, about 60%, about 70%, about 80%, or at least about 90% compared to an in vitro or in vivo cell (e.g., cancer cell) that is not being treated with a compound of Formulae (A), (B), (I), (II), (III) and/or (IV).

In some embodiments, the methods disclosed herein are directed to a decrease and/or reduction of PNC prevalence (e.g., the percentage of cells (e.g., cancer cell) containing at least 1 PNC), the method comprising contacting a single or plurality of cell(s), tissue, or organ with an effective amount of a compound of Formulae (A), (B), (I), (II), (III) and/or (IV) or salt thereof. In some embodiments such cell(s), tissue, or organs are cancerous. In some embodiments, the disclosure provides methods of decreasing and/or reducing PNC prevalence in a subject including but not limited to rodents and mammals, e.g., humans, by administering to the subject an effective amount of a compound of Formulae (A), (B), (I), (II), (III) and/or (IV) or salt thereof. Thus, the present disclosure is directed to methods of reducing and/or decreasing PNC prevalence in an in vitro and in vivo testing environment, which a skilled artisan would be familiar with. In some embodiments, the percentage of reduction and/or decrease in PNC prevalence in a single cell (e.g, cancer cell) or a plurality of cells in vitro and/or in vivo being at least about 10%, about 25%, about 30%, about 40%, about 50%, about 60%, about 70%, about 80%, or at least about 90% compared to an in vitro or in vivo cell(s) that is (are) not being treated with a compound of Formulae (I), (II) and/or (III).

The disclosure additionally provides a method for modulating the morphology of any PNC's in a cell, comprising contacting the cell (e.g., cancer cell) in vitro or in vivo with an effective amount of a compound Formulae (A), (B), (I), (II), (III) and/or (IV) or salt thereof. In some embodiments, the morphology of a cell (e.g., cancer cell) contacted with a compound Formulae (A), (B), (I), (II), (III) and/or (IV) or salt thereof changes from being spherical in shape to non-spherical in shape, e.g., sickle shaped.

The disclosure additionally provides a method for disrupting a PNC in a cell (e.g., cancer cell), comprising contacting the cell in vitro or in vivo with an effective amount of a compound Formulae (A), (B), (I), (II), (III) and/or (IV) or salt thereof. In some embodiments, the amount of PNCs being disrupted in a cell (e.g., cancer cell) ranges from at least 20% to about 90%, from at least 20% to about 80%, from at least 30% to about 70%, or from about 40% to about 50% compared based on the total amount of PNCs in a cell.

The compositions containing the compounds or salts thereof described herein can be administered for prophylactic and/or therapeutic treatments. In therapeutic applications, the compositions are administered to a patient already suffering from a disease, in an amount sufficient to cure or at least partially arrest the symptoms of the disease. Amounts effective for this use will depend on the severity and course of the disease, previous therapy, the patient's health status, weight, and response to the drugs, and the judgment of the treating clinician.

In prophylactic applications, compositions containing the compounds or salts thereof described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating clinician.

The amount of a given agent that will correspond to such an amount will vary depending upon factors such as the particular compound, disease and its severity, the identity (e.g., weight) of the subject or host in need of treatment, but can nevertheless be determined in a manner recognized in the field according to the particular circumstances surrounding the case, including e.g., the specific agent being administered, the route of administration, the condition being treated, and the subject or host being treated. In general, however, doses employed for adult human treatment will typically be in the range of about 0.02-about 5000 mg per day, in some embodiments, about 1-about 1500 mg per day. The desired dose may conveniently be presented in a single dose or as divided doses administered simultaneously (or over a short period of time) or at appropriate intervals, for example as two, three, four or more sub-doses per day.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in humans. The dosage of such compounds lies preferably within a range of circulating concentrations that include the ED₅₀ with minimal toxicity. The dosage may vary within this range depending upon the dosage form employed and the route of administration utilized.

### EXAMPLES

### Example 1: PNC Inhibitor Screening

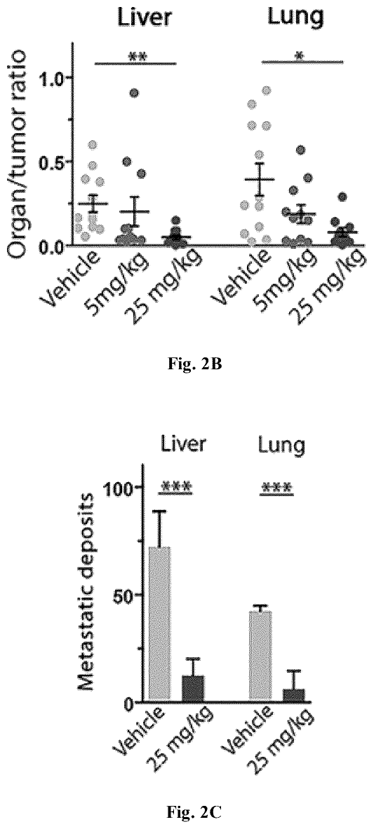

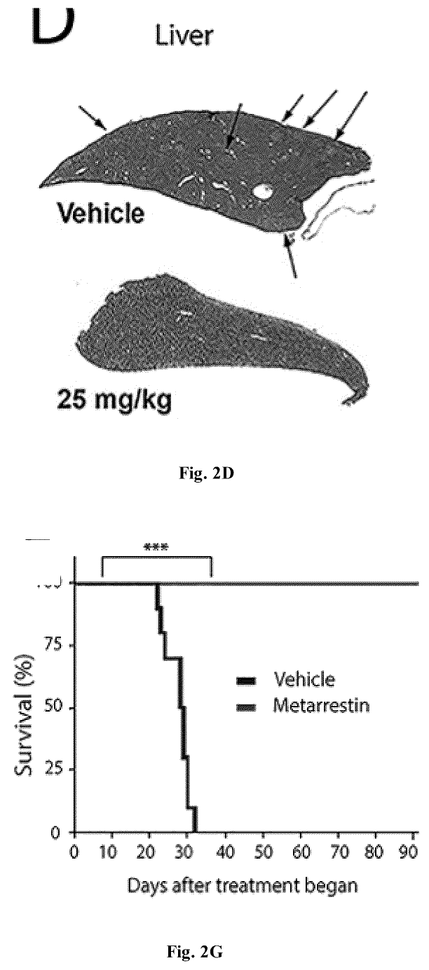

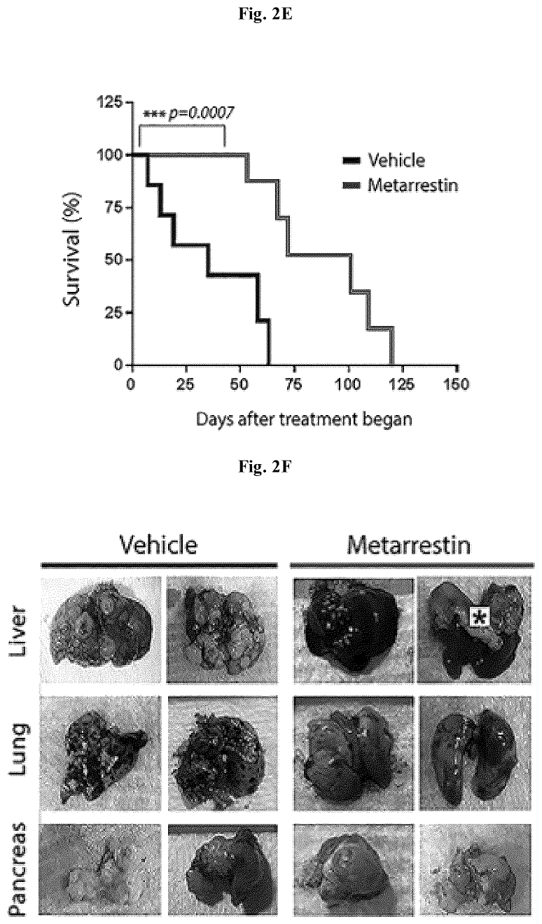

Using a high-content screen to measure reduction of PNC prevalence [27], noncytotoxic PNC inhibitor hit compounds were discovered such as metarrestin, (FIG. 2A) [28]. Metarrestin, a small molecule PNC inhibitor, showed in vitro anti-oncogenic properties including migration and invasion inhibition, and anti-metastatic activity in three in vivo models [5]. In an orthotopic mouse model utilizing 3-D cultured PANC1 cells, daily metarrestin treatment (25 mg/kg IP) for four weeks significantly reduced the metastatic burden in lung and liver (FIGS. 2B, 2C, and 2D) with only modest reduction of primary tumor growth (data not shown). Metarrestin was further evaluated in xenograft models of PC3M (metastatic prostate cancer cell line) and in a breast cancer patient-derived xenograft (PDX, harvested from a lung metastasis site). The results showed significant inhibition of organ metastasis in the PC3M xenograft and metastatic tumor growth inhibition in the PDX, further supporting metarrestin's selective inhibition of metastatic cancer cells [5]. When the survival of the PANC1 tumor-bearing animals was evaluated, two sets of experiments were performed to determine efficacy at two treatment starting points, either after (eight weeks post-inoculation) or prior to (four weeks post-inoculation) macrometastasis had been established. Metarrestin-containing food pellets (formulated to provide 10 mg/kg daily dosing) significantly improved animal survival when treatment started post-macrometastasis formation (FIG. 2F) and completely prevented metastasis-induced death when treatment started prior to macrometastasis formation (FIG. 2E). In the former group, the autopsy analyses showed that treated animals did not die from metastasis as their organs did not show significant tumor replacement compared to those fed regular food pellets (FIG. 2G) [5]. Metarrestin displays excellent oral bioavailability (F-95%), good tissue distribution and a long half-life that allows once-a-day dosing [29]. Daily treatment for up to three months does not induce discernable adverse effects in animals [5].

### Example 2. Synthesis of Representative Metarrestin Analogues IC-1-IC-5

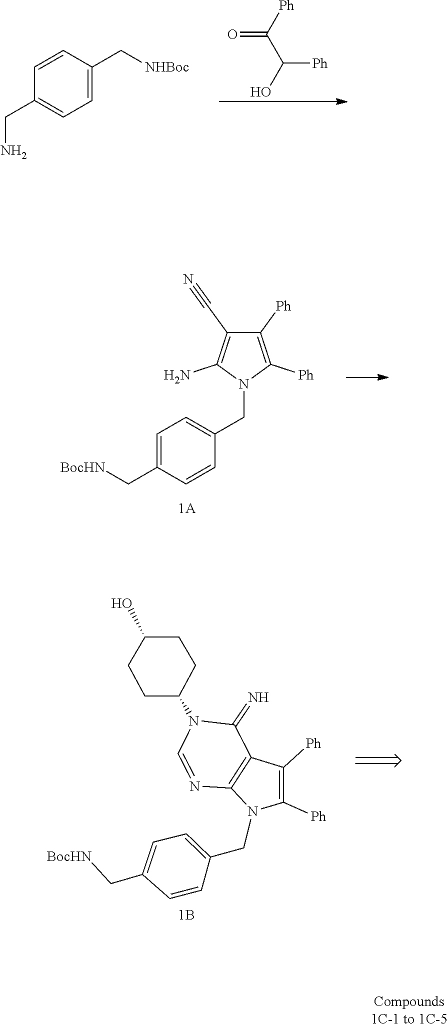 [SMILES: CC(C)(C)OC(=O)NCc1ccc(CN)cc1 ; O=C(c1ccccc1)C(O)c1ccccc1 ; CC(C)(C)OC(=O)NCc1ccc(Cn2c(N)c(C#N)c(-c3ccccc3)c2-c2ccccc2)cc1 ; CC(C)(C)OC(=O)NCc1ccc(Cn2c(-c3ccccc3)c(-c3ccccc3)c3c(=N)n([C@H]4CC[C@@H](O)CC4)cnc32)cc1]

### Preparation of tert-Butyl (4-((2-amino-3-cyano-4,5-diphenyl-1H-pyrrol-1-yl)methyl)benzyl)carbamate (1A)

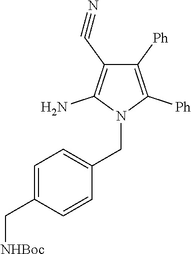 [SMILES: CC(C)(C)OC(=O)NCc1ccc(Cn2c(N)c(C#N)c(-c3ccccc3)c2-c2ccccc2)cc1]

A suspension of the tert-butyl (4-(aminomethyl)benzyl)carbamate (1.1823 g, 1 Eq, 5.0031 mmol) and acyloin 2-hydroxy-1,2-diphenylethan-1-one (1.0619 g, 1 Eq, 5.0031 mmol) in benzene (8 mL) was heated with microwave irradiation (120° C.) for 0.5 hour. Then, malononitrile (330.5 mg, 1 Eq, 5.0031 mmol) and potassium tert-butoxide (112.28 mg, 0.2 Eq, 1.0006 mmol) were 20 added, and heated with microwave irradiation (120° C.) for 1.0 hour. The solvent was removed in vacuo, and purified by silica chromatography (EtOAc/hexane: 0 to 100%) to afford the 2-amino-1H-pyrrole-3-carbonitrile derivative (0.2819 g, 589.0 μmol, 11.8% yield). ¹H NMR (400 MHz, CDCl₃) δ 1.46 (s, 9H), 3.89 (s, 2H), 4.31 (d, J=5.8 Hz, 2H), 4.89 (s, 2H), 7.03 (d, J=8.1 Hz, 2H), 7.53-7.08 (m, 12H).

### Preparation of tert-Butyl (4-((3-((trans)-4-hydroxycyclohexyl)-4-imino-5,6-diphenyl-3,4-dihydro-7H-pyrrolo[2,3-d]pyrimidin-7-yl)methyl)benzyl)carbamate (1B)

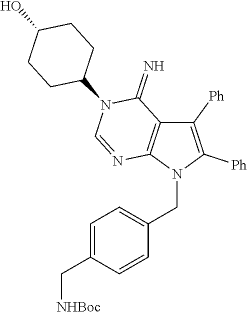

tert-Butyl (4-((2-amino-3-cyano-4,5-diphenyl-1H-pyrrol-1-yl)methyl)benzyl)carbamate (0.3527 g, 1 Eq, 736.9 μmol) in triethoxymethane (2.184 g, 20 Eq, 14.74 mmol) was refluxed for 4 h, and concentrated to dryness. The resulting imido ester was treated with (trans)-4-aminocyclohexan-1-ol (169.8 mg, 2 Eq, 1.474 mmol), and potassium tert-butoxide (41.35 mg 0.5 Eq, 368.5 μmol) in MeOH (20 mL) was heated at 50° C. for 24 h, and then cooled to room temperature and filtered. The solvent was removed in vacuo, and purified by silica chromatography (methanol/DCM: 0 to 30%) to afford the pyrrolopyrimidine product (196.8 mg, 326.0 μmol, 44.2% yield). ¹H NMR (400 MHz, CDCl₃) δ 1.43 (s, 9H), 1.71-1.47 (m, 4H), 2.06-2.08 (m, 4H), 2.63 (s, 2H), 3.64 (dd, J=12.1, 8.4 Hz, 1H), 4.23 (d, J=5.0 Hz, 2H), 4.92 (s, 1H), 5.02 (t, J=11.7 Hz, 1H), 5.24 (s, 2H), 6.43 (s, 1H), 6.90 (d, J=8.1 Hz, 2H), 6.99-7.05 (m, 2H), 7.12 (d, J=8.0 Hz, 2H), 7.26-7.15 (m, 8H), 7.73 (s, 1H). ¹³C NMR (101 MHz, CDCl₃) δ 155.78, 155.30, 142.39, 142.19, 137.93, 136.87, 133.69, 132.69, 130.92, 130.44, 128.17, 128.09, 127.91, 127.43, 126.92, 126.79, 118.11, 103.10, 79.39, 69.56, 51.61, 45.65, 44.16, 34.63, 30.46, 28.30.

### Preparation of 3-(2-((2-(2,6-Dioxopiperidin-3-yl)-1,3-dioxoisoindolin-4-yl)amino)ethoxy)-N-(4-((3-((trans)-4-hydroxycyclohexyl)-4-imino-5,6-diphenyl-3,4-dihydro-7H-pyrrolo[2,3-d]pyrimidin-7-yl)methyl)benzyl)propanamide (1C-1)

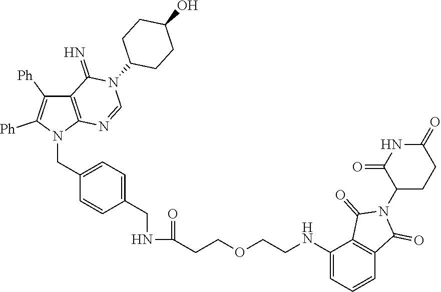

tert-Butyl (4-((3-((trans)-4-hydroxycyclohexyl)-4-imino-5,6-diphenyl-3,4-dihydro-7H-pyrrolo[2,3-d]pyrimidin-7-yl)methyl)benzyl)carbamate (16.3 mg, 1 Eq, 27.0 μmol) was treated with TFA/DCM (1/1, 1 mL), and stirred for 1 hour. The solvent was removed in vacuo, and N-ethyl-N-isopropylpropan-2-amine (13.9 mg, 4 Eq, 108 μmol) and 3-(2-((2-(2,6-dioxopiperidin-3-yl)-1,3-dioxoisoindolin-4-yl)amino)ethoxy)propanoic acid (10.5 mg, 1 Eq, 27.0 μmol) were added. Then, HATU (10.3 mg, 1 Eq, 27.0 μmol) was added to the above stirred reaction solution, and stirred at room temperature for 2 hours. DMF was removed under a stream of N₂ and the reaction mixture was purified by flash chromatography on silica gel (methanol/DCM: 0 to 30%) to give the desired product (7.4 mg, 8.5 μmol, 31% yield). ¹H NMR (400 MHz, CDCl₃) δ 1.70-1.85 (m, 4H), 1.98-2.09 (m, 3H), 2.20-2.22 (m, 2H), 2.50 (t, J=5.7 Hz, 2H), 2.62-2.70 (m, 2H), 2.74-2.82 (m, 1H), 3.38 (q, J=5.3 Hz, 2H), 3.62-3.70 (m, 3H), 3.77 (t, J=5.7 Hz, 2H), 4.37 (d, J=5.9 Hz, 2H), 4.76-4.83 (m, 1H), 5.28-5.35 (m, 3H), 6.45 (t, J=5.5 Hz, 1H), 6.73 (t, J=5.8 Hz, 1H), 6.79-6.84 (m, 3H), 6.99-7.19 (m, 7H), 7.19-7.32 (m, 6H), 7.38-7.44 (m, 1H), 8.08 (d, J=7.9 Hz, 1H), 8.13 (s, 1H), 8.27 (d, J=3.7 Hz, 1H). ¹³C NMR (101 MHz, CDCl₃) δ 22.6, 31.1, 31.4, 33.9, 37.3, 42.1, 42.8, 46.2, 48.9, 52.8, 67.3, 69.2, 69.3, 101.1, 110.3, 111.8, 116.8, 117.3, 118.7, 127.0, 127.5, 127.6, 128.1, 128.5, 129.0, 129.0, 130.3, 130.8, 131.7, 132.4, 135.3, 135.5, 136.1, 138.3, 139.5, 141.4, 144.6, 146.7, 147.1, 152.6, 167.5, 168.6, 169.5, 171.2, 171.4.

### Preparation of 6-((2-(2,6-Dioxopiperidin-3-yl)-1,3-dioxoisoindolin-4-yl)amino)-N-(4-((3-((trans)-4-hydroxycyclohexyl)-4-imino-5,6-diphenyl-3,4-dihydro-7H-pyrrolo[2,3-d]pyrimidin-7-yl)methyl)benzyl)hexanamide (1C-2)

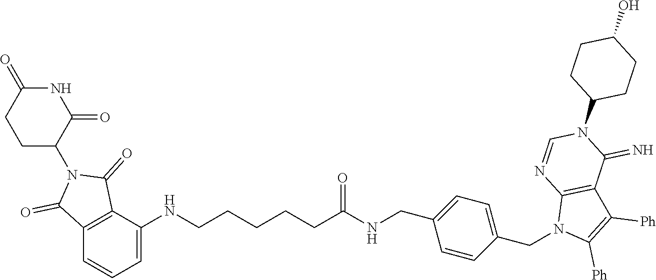

tert-Butyl (4-((3-((trans)-4-hydroxycyclohexyl)-4-imino-5,6-diphenyl-3,4-dihydro-7H-pyrrolo[2,3-d]pyrimidin-7-yl)methyl)benzyl)carbamate (17.6 mg, 1 Eq, 29.2 μmol) was treated with TFA/DCM (1/1, 1 mL), and stirred for 1 hour. The solvent was removed in vacuo, and N-ethyl-N-isopropylpropan-2-amine (15.1 mg, 4 Eq, 117 μmol) and 6-((2-(2,6-dioxopiperidin-3-yl)-1,3-dioxoisoindolin-4-yl)amino)hexanoic acid (11.3 mg, 1 Eq, 29.2 μmol) were added. Then, HATU (11.1 mg, 1 Eq, 29.2 μmol) was added to the above stirred reaction solution, and stirred at room temperature for 2 hours. DMF was removed under a stream of N₂, and the reaction mixture was purified by flash chromatography on silica gel (methanol/DCM: 0 to 30%) and on C18 media (methanol/water) to give the desired product (8.9 mg, 10 μmol, 35% yield). ¹H NMR (400 MHz, CDCl₃) δ 1.44 (dt, J=15.3, 7.7 Hz, 34H), 1.70 (dq, J=14.2, 7.5 Hz, 179H), 2.04-2.14 (m, 124H), 2.21 (t, J=7.4 Hz, 43H), 2.67-2.87 (m, 69H), 3.25 (q, J=6.7 Hz, 48H), 3.44 (d, J=18.0 Hz, 11H), 3.67 (s, 1H), 4.31-4.44 (m, 43H), 4.84-4.89 (m, 23H), 5.18 (s, 1H), 5.28 (s, 2H), 5.94 (s, 1H), 6.21 (t, J=5.6 Hz, 1H), 6.84-6.95 (m, 4H), 7.03-7.08 (m, 3H), 7.13 (d, J=8.1 Hz, 2H), 7.19-7.28 (m, 6H), 7.44-7.49 (m, 1H), 7.87 (s, 1H). ¹³C NMR (101 MHz, CDCl₃) δ−0.3, 22.7, 23.1, 25.2, 26.5, 28.9, 29.3, 29.4, 29.6, 29.6, 30.7, 31.4, 31.9, 33.4, 34.5, 36.4, 42.4, 43.1, 45.9, 48.9, 69.7, 76.7, 77.0, 77.3, 109.9, 111.5, 116.6, 127.1, 127.5, 127.9, 127.9, 128.3, 128.6, 130.5, 130.9, 132.4, 136.1, 136.8, 137.6, 142.2, 146.9, 167.6, 168.5, 169.5, 171.2, 172.6.

### Preparation of 3-(2-(2-(2-((2-(2,6-Dioxopiperidin-3-yl)-1,3-dioxoisoindolin-4-yl)amino)ethoxy)ethoxy)ethoxy)-N-(4-((3-((trans)-4-hydroxycyclohexyl)-4-imino-5,6-diphenyl-3,4-dihydro-7H-pyrrolo[2,3-d]pyrimidin-7-yl)methyl)benzyl)propanamide (1C-3)

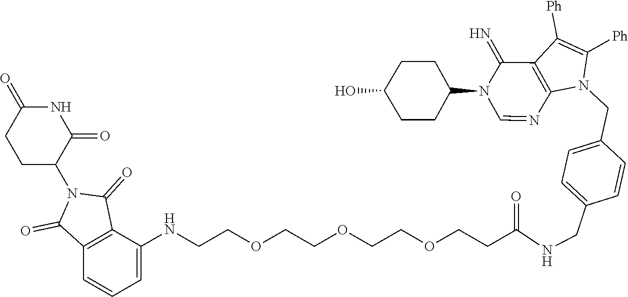

tert-Butyl (4-((3-((trans)-4-hydroxycyclohexyl)-4-imino-5,6-diphenyl-3,4-dihydro-7H-pyrrolo[2,3-d]pyrimidin-7-yl)methyl)benzyl)carbamate (14.4 mg, 1 Eq, 23.9 μmol) was treated with TFA/DCM (1/1, 1 mL), and stirred for 1 hour. The solvent was removed in vacuo, and N-ethyl-N-isopropylpropan-2-amine (12.3 mg, 4 Eq, 95.5 μmol) and 3-(2-(2-(2-((2-(2,6-dioxopiperidin-3-yl)-1,3-dioxoisoindolin-4-yl)amino)ethoxy)ethoxy)ethoxy)propanoic acid (11.4 mg, 1 Eq, 23.9 μmol) were added. Then, HATU (9.08 mg, 1 Eq, 23.9 μmol) was added to the above stirred reaction solution, and stirred at room temperature for 2 hours. DMF was removed under a stream of N₂, and the reaction mixture was purified by flash chromatography on silica gel (methanol/DCM: 0 to 35% yield) to give the desired product (8.3 mg, 8.6 μmol, 36%). ¹H NMR (400 MHz, CDCl₃) δ 1.39-1.45 (m, 4H), 1.69-1.91 (m, 4H), 2.06-2.12 (m, 3H), 2.26 (d, J=9.7 Hz, 2H), 2.49 (t, J=5.8 Hz, 2H), 2.59-2.92 (m, 3H), 3.10 (q, J=7.4 Hz, 1H), 3.31-3.49 (m, 2H), 3.49-3.87 (m, 11H), 4.37 (d, J=5.7 Hz, 2H), 4.87 (dd, J=11.4, 5.3 Hz, 1H), 5.32-5.49 (m, 3H), 6.45 (t, J=5.3 Hz, 1H), 6.76-6.93 (m, 3H), 6.98-7.37 (m, 12H), 7.46 (t, J=8.0 Hz, 1H), 8.03-8.21 (m, 2H), 8.32 (s, 1H). ¹³C NMR (101 MHz, CDCl₃) δ 22.8, 31.2, 31.4, 33.9, 36.8, 41.4, 42.3, 42.8, 46.3, 48.9, 52.8, 67.1, 69.4, 70.1, 70.4, 70.4, 70.7, 101.0, 110.3, 111.7, 116.7, 117.3, 118.8, 127.2, 127.5, 127.7, 128.2, 128.5, 129.0, 129.1, 130.3, 130.8, 132.5, 135.3, 135.4, 136.0, 138.4, 139.5, 141.1, 144.7, 146.7, 147.2, 152.5, 167.5, 168.5, 169.3, 171.3, 171.6.

### Preparation of 4-((2-(2,6-Dioxopiperidin-3-yl)-1,3-dioxoisoindolin-4-yl)amino)-N-(4-((3-((trans)-4-hydroxycyclohexyl)-4-imino-5,6-diphenyl-3,4-dihydro-7H-pyrrolo[2,3-d]pyrimidin-7-yl)methyl)benzyl)butanamide (1C-4)

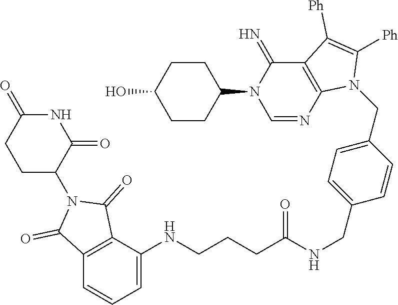

tert-Butyl (4-((3-((trans)-4-hydroxycyclohexyl)-4-imino-5,6-diphenyl-3,4-dihydro-7H-pyrrolo[2,3-d]pyrimidin-7-yl)methyl)benzyl)carbamate (17.0 mg, 1 Eq, 28.1 μmol) was treated with TFA/DCM (1/1, 1 mL), and stirred for 1 hour. The solvent was removed in vacuo, and N-ethyl-N-isopropylpropan-2-amine (14.5 mg, 4 Eq, 112 μmol) and 4-((2-(2,6-dioxopiperidin-3-yl)-1,3-dioxoisoindolin-4-yl)amino)butanoic acid (10.1 mg, 1 Eq, 28.1 μmol) were added. Then, HATU (10.7 mg, 1 Eq, 28.1 μmol) was added to the above stirred reaction solution, and stirred at room temperature for 2 hours. DMF was removed under a stream of N₂, and the reaction mixture was purified by flash chromatography on silica gel (methanol/DCM: 0 to 35%) to give the desired product (9.7 mg, 11 μmol, 41% yield). ¹H NMR (400 MHz, CDCl₃) δ 1.34-1.45 (m, 4H), 1.64-1.88 (m, 4H), 1.90-2.13 (m, 5H), 2.19-2.22 (m, 2H), 2.31 (t, J=7.0 Hz, 2H), 2.60-2.90 (m, 3H), 3.09 (q, J=7.2 Hz, 2H), 3.31 (q, J=6.3 Hz, 2H), 3.47 (s, 1H), 3.58 (br s, 1H), 3.70 (dt, J=13.1, 6.7 Hz, 2H), 4.36 (d, J=5.5 Hz, 2H), 4.88 (dd, J=12.0, 5.5 Hz, 1H), 5.29-5.34 (m, 3H), 6.25 (t, J=5.9 Hz, 2H), 6.58 (br s, 1H), 6.84-6.88 (m, 3H), 6.95-7.16 (m, 8H), 7.20-7.36 (m, 6H), 7.41 (t, J=8.0 Hz, 1H), 8.07-8.19 (m, 2H), 8.32 (d, J=3.9 Hz, 1H). ¹³C NMR (101 MHz, CDCl₃) δ 22.7, 24.9, 31.1, 31.4, 33.1, 33.9, 41.4, 41.8, 43.0, 46.3, 48.9, 50.7, 52.8, 69.3, 101.1, 110.0, 111.5, 116.8, 117.3, 118.8, 127.2, 127.5, 128.0, 128.2, 128.5, 129.0, 129.1, 130.3, 130.8, 132.4, 135.3, 135.6, 136.1, 138.2, 139.5, 141.2, 146.8, 147.2, 167.5, 168.7, 169.5, 171.4, 172.0.

### Preparation of 3-(2-(2-((2-(2,6-Dioxopiperidin-3-yl)-1,3-dioxoisoindolin-4-yl)amino)ethoxy)ethoxy)-N-(4-((3-((trans)-4-hydroxycyclohexyl)-4-imino-5,6-diphenyl-3,4-dihydro-7H-pyrrolo[2,3-d]pyrimidin-7-yl)methyl)benzyl)propanamide (1C-5)

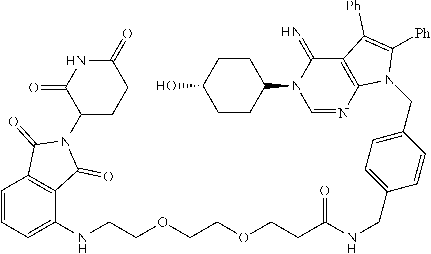

tert-Butyl (4-((3-((trans)-4-hydroxycyclohexyl)-4-imino-5,6-diphenyl-3,4-dihydro-7H-pyrrolo[2,3-d]pyrimidin-7-yl)methyl)benzyl)carbamate (16.6 mg, 1 Eq, 27.5 μmol) was treated with TFA/DCM (1/1, 1 mL), and stirred for 1 hour. The solvent was removed in vacuo, and N-ethyl-N-isopropylpropan-2-amine (14.2 mg, 4 Eq, 110 μmol) and 3-(2-(2-((2-(2,6-dioxopiperidin-3-yl)-1,3-dioxoisoindolin-4-yl)amino)ethoxy)ethoxy)propanoic acid (11.9 mg, 1 Eq, 27.5 μmol) were added. Then, HATU (10.4 mg, 1 Eq, 27.5 μmol) was added to the above stirred reaction solution, and stirred at room temperature for 2 hours. DMF was removed under a stream of N₂, and the reaction mixture was purified by flash chromatography on silica gel (methanol/DCM. 0 to 35%) to give the desired product (10.1 mg, 11.0 μmol, 40.0% yield). ¹H NMR (400 MHz, CDCl₃) δ 1.03-1.50 (m, 4H), 1.75 (q, J=10.9, 10.4 Hz, 4H), 1.99-2.28 (m, 4H), 2.51 (t, J=5.8 Hz, 2H), 2.58-2.85 (m, 2H), 3.34 (q, J=5.2 Hz, 2H), 3.46 (s, 1H), 3.52-3.71 (m, 6H), 3.76 (t, J=5.8 Hz, 2H), 4.37 (d, J=5.8 Hz, 2H), 4.78 (dd, J=12.1, 5.3 Hz, 1H), 5.23-5.37 (m, 2H), 6.44 (t, J=5.3 Hz, 1H), 6.81-6.90 (m, 4H), 6.99-7.32 (m, 12H), 7.42-7.47 (m, 1H), 8.03 (s, 1H), 8.10 (d, J=8.3 Hz, 1H), 8.29 (d, J=4.0 Hz, 1H). ¹³C NMR (101 MHz, CDCl₃) δ 22.7, 31.0, 31.4, 34.1, 37.0, 42.2, 42.8, 46.1, 48.9, 50.7, 67.3, 69.1, 69.5, 70.0, 70.3, 101.6, 110.3, 111.7, 116.7, 117.6, 118.7, 127.0, 127.5, 127.6, 127.9, 128.4, 128.8, 128.9, 130.4, 130.9, 132.5, 135.3, 135.9, 136.1, 138.2, 139.5, 141.6, 146.7, 147.0, 167.5, 168.5, 169.4, 171.2, 171.7.

### Example 3. Synthesis of Representative Metarrestin Analogues 2C-1 to 2C-5

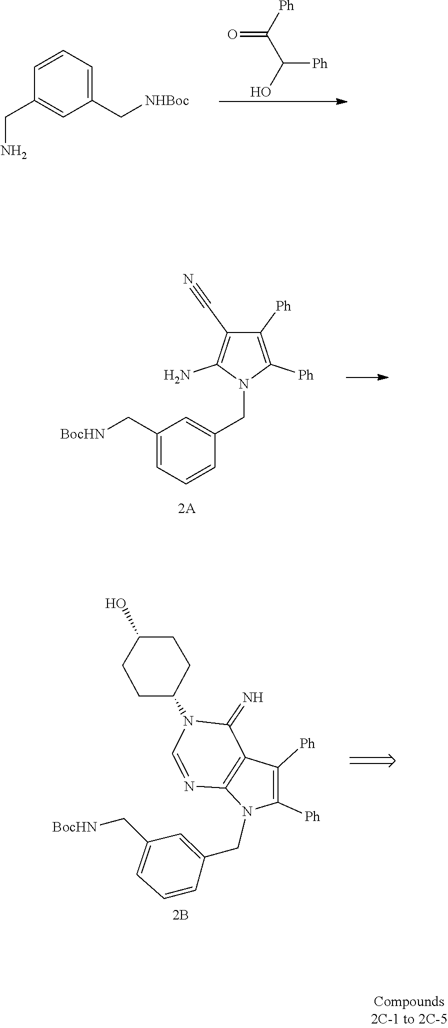 [SMILES: CC(C)(C)OC(=O)NCc1cccc(CN)c1 ; O=C(c1ccccc1)C(O)c1ccccc1 ; CC(C)(C)OC(=O)NCc1cccc(Cn2c(N)c(C#N)c(-c3ccccc3)c2-c2ccccc2)c1 ; CC(C)(C)OC(=O)NCc1cccc(Cn2c(-c3ccccc3)c(-c3ccccc3)c3c(=N)n([C@H]4CC[C@@H](O)CC4)cnc32)c1]

### Preparation of tert-Butyl (3-((2-amino-3-cyano-4,5-diphenyl-1H-pyrrol-1-yl)methyl)benzyl)carbamate (2A)

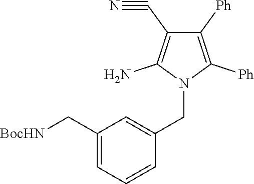 [SMILES: CC(C)(C)OC(=O)NCc1cccc(Cn2c(N)c(C#N)c(-c3ccccc3)c2-c2ccccc2)c1]

A suspension of the tert-butyl (3-(aminomethyl)benzyl)carbamate (1.2048 g, 1 Eq, 5.0982 mmol) and acyloin 2-hydroxy-1,2-diphenylethan-1-one (1.0821 g, 1 Eq, 5.0982 mmol) in benzene (8 mL) was heated with microwave irradiation (120° C.) for 0.5 hour. Then, malononitrile (336.8 mg, 1 Eq, 5.0982 mmol) and potassium tert-butoxide (114.41 mg, 0.2 Eq, 1.0196 mmol) were added, and heated with microwave irradiation (120° C.) for 1.0 hour. The solvent was removed in vacuo, and purified by silica chromatography (EtOAc/hexane: 0 to 100%) to afford the 2-amino-1H-pyrrole-3-carbonitrile derivative (0.2933 g, 612.8 μmol, 12.0% yield). ¹H NMR (400 MHz, CDCl₃) δ 1.46 (s, 9H), 3.99 (s, 2H), 4.30 (d, J=5.3 Hz, 2H), 4.91 (s, 2H), 6.96 (d, J=7.6 Hz, 1H), 7.01 (s, 1H), 7.38-7.13 (m, 13H). ¹³C NMR (101 MHz, CDCl₃) δ 28.3, 44.2, 46.9, 75.7, 79.7, 117.5, 121.0, 124.7, 124.8, 125.6, 126.4, 126.8, 128.1, 128.1, 128.6, 128.7, 129.5, 130.8, 131.0, 133.1, 136.5, 140.2, 146.1, 176.0.

### Preparation of tert-Butyl (3-((3-((trans)-4-hydroxycyclohexyl)-4-imino-5,6-diphenyl-3,4-dihydro-7H-pyrrolo[2,3-d]pyrimidin-7-yl)methyl)benzyl)carbamate (2B)

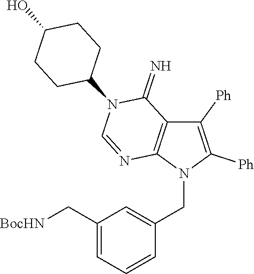

tert-Butyl (3-((2-amino-3-cyano-4,5-diphenyl-1H-pyrrol-1-yl)methyl)benzyl)carbamate (0.2763 g, 1 Eq, 577.3 μmol) in triethoxymethane (1.711 g, 20 Eq, 11.55 mmol) was refluxed for 4 h, and concentrated to dryness. The resulting imido ester was treated with (trans)-4-aminocyclohexan-1-ol (133.0 mg, 2 Eq, 1.155 mmol), and potassium tert-butoxide (32.39 mg 0.5 Eq, 288.7 μmol) in MeOH (20 mL) was heated at 50° C. for 24 h, and then cooled to room temperature and filtered. The solvent was removed in vacuo, and purified by silica chromatography (methanol/DCM: 0 to 30%) to afford the pyrrolopyrimidine product (131.1 mg, 217.1 μmol, 37.6% yield). ¹H NMR (400 MHz, CDCl₃) δ 7.85 (s, 1H), 7.31-7.08 (m, 9H), 7.07-6.96 (m, 2H), 6.89-6.75 (m, 2H), 5.27 (s, 2H), 5.17 (s, 1H), 4.77 (s, 1H), 4.18 (d, J=5.4 Hz, 2H), 3.66 (d, J=4.0 Hz, 1H), 2.12 (br s, 4H), 1.62-1.73 (m, 4H), 1.43 (s, 9H). ¹³C NMR (101 MHz, CDCl₃) δ 155.70, 154.40, 142.98, 142.00, 139.06, 137.69, 134.06, 133.00, 130.96, 130.42, 130.10, 128.81, 128.48, 128.28, 128.22, 127.25, 126.50, 126.03, 125.93, 117.90, 102.41, 79.45, 69.55, 53.09, 46.02, 44.38, 34.40, 30.71, 28.35.

### Preparation of 6-((2-(2,6-Dioxopiperidin-3-yl)-1,3-dioxoisoindolin-4-yl)amino)-N-(3-((3-((trans)-4-hydroxycyclohexyl)-4-imino-5,6-diphenyl-3,4-dihydro-7H-pyrrolo[2,3-d]pyrimidin-7-yl)methyl)benzyl)hexanamide (2C-1)

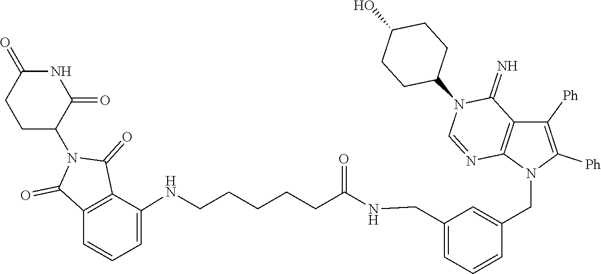

tert-Butyl (3-((3-((trans)-4-hydroxycyclohexyl)-4-imino-5,6-diphenyl-3,4-dihydro-7H-pyrrolo[2,3-d]pyrimidin-7-yl)methyl)benzyl)carbamate (19.2 mg, 1 Eq, 31.8 μmol) was treated with TFA/DCM (1/1, 1 mL), and stirred for 1 hour. The solvent was removed in vacuo, and N-ethyl-N-isopropylpropan-2-amine (16.4 mg, 4 Eq, 127 μmol) and 6-((2-(2,6-dioxopiperidin-3-yl)-1,3-dioxoisoindolin-4-yl)amino)hexanoic acid (12.3 mg, 1 Eq, 31.8 μmol) were added. Then, HATU (12.1 mg, 1 Eq, 31.8 μmol) was added to the above stirred reaction solution, and stirred at room temperature for 2 hours. DMF was removed under a stream of N₂, and the reaction mixture was purified by flash chromatography on silica gel (methanol/DCM: 0 to 25%) to give the desired product (13.1 mg, 15.0 μmol, 47.3%). ¹H NMR (400 MHz, CDCl₃) δ 8.87 (s, 1H), 8.28 (s, 1H), 7.43 (dd, J=8.4, 7.2 Hz, 1H), 7.34-7.22 (m, 4H), 7.18 (dd, J=6.5, 2.9 Hz, 2H), 7.11 (d, J=4.8 Hz, 2H), 7.02 (t, J=7.6 Hz, 2H), 6.81-6.85 (m, 2H), 6.68 (t, J=4.0 Hz, 1H), 6.57 (br s, 1H), 6.17 (s, 1H), 5.33 (s, 2H), 4.99-4.79 (m, 2H), 4.26 (d, J=5.8 Hz, 2H), 3.65 (br s, 3H), 3.41 (s, 2H), 3.21 (br s, 2H), 2.66-2.81 (m, 3H), 2.16-2.10 (m, 3H), 2.06-2.10 (m, 2H), 1.58-1.86 (m, 6H), 1.35-1.46 (m, 2H). ¹³C NMR (101 MHz, CDCl₃) δ 172.88, 171.38, 169.41, 168.63, 167.54, 151.41, 146.84, 145.47, 140.68, 139.39, 138.57, 136.18, 136.06, 132.33, 130.97, 130.73, 130.21, 129.34, 129.28, 128.84, 128.57, 128.39, 127.12, 126.55, 125.84, 121.07, 118.16, 116.85, 116.65, 115.24, 112.32, 111.23, 109.72, 100.32, 68.67, 57.51, 50.36, 48.78, 46.61, 42.92, 42.30, 36.08, 33.43, 31.31, 30.79, 28.80, 26.42, 25.18, 22.65.

### Preparation of 3-(2-((2-(2,6-Dioxopiperidin-3-yl)-1,3-dioxoisoindolin-4-yl)amino)ethoxy)-N-(3-((3-((trans)-4-hydroxycyclohexyl)-4-imino-5,6-diphenyl-3,4-dihydro-7H-pyrrolo[2,3-d]pyrimidin-7-yl)methyl)benzyl)propanamide (2C-2)

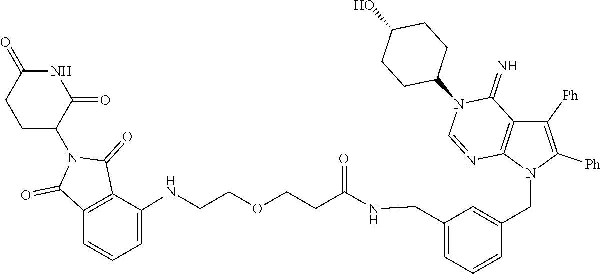

tert-Butyl (3-((3-((trans)-4-hydroxycyclohexyl)-4-imino-5,6-diphenyl-3,4-dihydro-7H-pyrrolo[2,3-d]pyrimidin-7-yl)methyl)benzyl)carbamate (17.5 mg, 1 Eq, 29.0 μmol) was treated with TFA/DCM (1/1, 1 mL), and stirred for 1 hour. The solvent was removed in vacuo, and N-ethyl-N-isopropylpropan-2-amine (15.0 mg, 4 Eq, 116 μmol) and 3-(2-((2-(2,6-dioxopiperidin-3-yl)-1,3-dioxoisoindolin-4-yl)amino)ethoxy)propanoic acid (11.3 mg, 1 Eq, 29.0 μmol) were added. Then, HATU (11.0 mg, 1 Eq, 29.0 μmol) was added to the above stirred reaction solution, and stirred at room temperature for 2 hours. DMF was removed under a stream of N₂, and the reaction mixture was purified by flash chromatography on silica gel (methanol/DCM: 0 to 25%) to give the desired product (12.7 mg, 14.5 μmol, 50.0% yield). ¹H NMR (400 MHz, CDCl₃) δ 1.35-1.46 (m, 4H), 1.70-1.87 (m, 4H), 1.98-2.31 (m, 5H), 2.49 (t, J=5.7 Hz, 2H), 2.57-2.84 (m, 3H), 3.41 (t, J=5.2 Hz, 2H), 3.68 (t, J=5.2 Hz, 2H), 3.74-3.82 (m, 2H), 4.12 (br s, 4H), 4.24-4.42 (m, 2H), 4.81 (dd, J=11.9, 5.2 Hz, 1H), 5.03 (br s, 1H), 6.47 (br s, 1H), 6.54-6.67 (m, 2H), 6.84-6.93 (m, 2H), 7.01-7.23 (m, 6H), 7.23-7.38 (m, 6H), 7.45 (dd, J=8.5, 7.2 Hz, 1H), 8.33 (s, 1H), 8.73 (s, 1H). ¹³C NMR (101 MHz, CDCl₃) δ 22.67, 30.92, 31.39, 33.59, 37.39, 42.13, 42.95, 46.65, 48.89, 57.61, 67.26, 69.01, 69.14, 100.48, 110.31, 111.88, 115.19, 116.87, 117.02, 118.10, 125.64, 126.38, 126.89, 128.40, 128.66, 128.76, 128.87, 129.42, 130.26, 130.80, 130.92, 132.39, 136.19, 136.32, 138.65, 139.36, 140.96, 145.38, 146.77, 151.55, 167.42, 168.51, 169.51, 171.18.

### Preparation of 3-(2-(2-((2-(2,6-Dioxopiperidin-3-yl)-1,3-dioxoisoindolin-4-yl)amino)ethoxy)ethoxy)-N-(3-((3-((trans)-4-hydroxycyclohexyl)-4-imino-5,6-diphenyl-3,4-dihydro-7H-pyrrolo[2,3-d]pyrimidin-7-yl)methyl)benzyl)propanamide (2C-3)

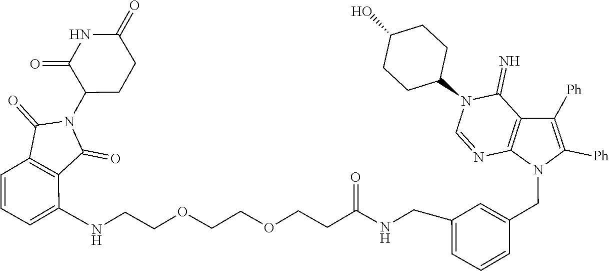

tert-Butyl (3-((3-((trans)-4-hydroxycyclohexyl)-4-imino-5,6-diphenyl-3,4-dihydro-7H-pyrrolo[2,3-d]pyrimidin-7-yl)methyl)benzyl)carbamate (16.6 mg, 1 Eq, 27.5 μmol) was treated with TFA/DCM (1/1, 1 mL), and stirred for 1 hour. The solvent was removed in vacuo, and N-ethyl-N-isopropylpropan-2-amine (14.2 mg, 4 Eq, 110 μmol) and 3-(2-(2-((2-(2,6-dioxopiperidin-3-yl)-1,3-dioxoisoindolin-4-yl)amino)ethoxy)ethoxy)propanoic acid (11.9 mg, 1 Eq, 27.5 μmol) were added. Then, HATU (10.4 mg, 1 Eq, 27.5 μmol) was added to the above stirred reaction solution, and stirred at room temperature for 2 hours. DMF was removed under a stream of N₂, and the reaction mixture was purified by flash chromatography on silica gel (methanol/DCM. 0 to 35%) to give the desired product (8.5 mg, 9.2 μmol, 34% yield). ¹H NMR (400 MHz, CDCl₃) δ 1.35-1.46 (m, 4H), 1.63-1.81 (m, 4H), 1.96-2.25 (m, 5H), 2.49 (t, J=5.9 Hz, 2H), 2.55-2.84 (m, 3H), 3.06 (s, 1H), 3.34 (q, J=5.3 Hz, 2H), 3.47-3.80 (m, 8H), 4.32 (qd, J=15.2, 5.9 Hz, 2H), 4.80 (dd, J=12.1, 5.3 Hz, 1H), 6.44 (t, J=5.5 Hz, 1H), 6.68-6.91 (m, 4H), 6.97-7.40 (m, 13H), 7.46 (dd, J=8.5, 7.2 Hz, 1H), 8.02 (br s, 1H), 8.10 (dd, J=8.4, 1.4 Hz, 1H), 8.29-8.41 (m, 1H). ¹³C NMR (101 MHz, CDCl₃) δ 22.74, 30.89, 31.37, 34.23, 37.01, 42.18, 42.94, 46.26, 48.85, 67.29, 69.09, 69.56, 70.02, 70.34, 110.27, 111.71, 116.70, 117.64, 118.67, 125.64, 126.05, 126.52, 127.46, 127.75, 128.39, 128.68, 128.82, 129.51, 130.39, 130.91, 132.48, 135.34, 136.08, 137.37, 139.13, 139.51, 141.81, 146.68, 147.01, 167.51, 168.58, 169.39, 171.19, 171.52. 22

### Preparation of 4-((2-(2,6-Dioxopiperidin-3-yl)-1,3-dioxoisoindolin-4-yl)amino)-N-(3-((3-((trans)-4-hydroxycyclohexyl)-4-imino-5,6-diphenyl-3,4-dihydro-7H-pyrrolo[2,3-d]pyrimidin-7-yl)methyl)benzyl)butanamide (2C-4)

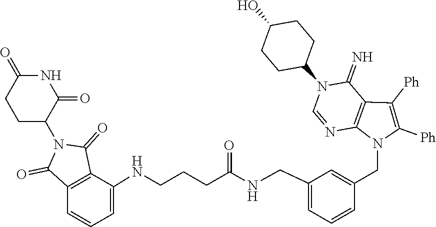

tert-Butyl (3-((3-((trans)-4-hydroxycyclohexyl)-4-imino-5,6-diphenyl-3,4-dihydro-7H-pyrrolo[2,3-d]pyrimidin-7-yl)methyl)benzyl)carbamate (18.7 mg, 1 Eq, 30.9 μmol) was treated with TFA/DCM (1/1, 1 mL), and stirred for 1 hour. The solvent was removed in vacuo, and N-ethyl-N-isopropylpropan-2-amine (16.0 mg, 4 Eq, 124 μmol) and 4-((2-(2,6-dioxopiperidin-3-yl)-1,3-dioxoisoindolin-4-yl)amino)butanoic acid (11.1 mg, 1 Eq, 30.9 μmol) were added. Then, HATU (11.7 mg, 1 Eq, 30.9 μmol) was added to the above stirred reaction solution, and stirred at room temperature for 2 hours. DMF was removed under a stream of N₂, and the reaction mixture was purified by flash chromatography on silica gel (methanol/DCM: 0 to 35%) to give the desired product (9.8 mg, 12 μmol, 38% yield). ¹H NMR (400 MHz, CDCl₃) δ 1.38-1.44 (m, 4H), 1.65-1.79 (m, 4H), 1.89-2.21 (m, 6H), 2.30 (t, J=7.1 Hz, 2H), 2.59-2.86 (m, 3H), 3.07 (q, J=7.5 Hz, 2H), 3.30 (q, J=6.5 Hz, 2H), 3.60-3.68 (m, 2H), 4.31 (d, J=5.7 Hz, 2H), 4.88 (dd, J=11.9, 5.4 Hz, 1H), 5.14 (br s, 1H), 5.30 (s, 2H), 6.26 (t, J=5.9 Hz, 1H), 6.49 (br s, 1H), 6.73 (d, J=6.8 Hz, 1H), 6.82-6.94 (m, 2H), 6.97-7.33 (m, 13H), 7.42 (dd, J=8.5, 7.1 Hz, 1H), 8.06 (d, J=8.5 Hz, 1H), 8.10 (s, 1H), 8.31 (d, J=4.2 Hz, 1H). ¹³C NMR (101 MHz, CDCl₃) δ 22.74, 24.88, 30.85, 31.41, 33.07, 34.01, 41.49, 41.86, 43.28, 46.35, 48.90, 69.27, 101.36, 110.00, 111.58, 116.83, 117.44, 118.75, 125.91, 126.51, 127.17, 127.44, 128.00, 128.47, 128.92, 128.96, 129.23, 130.37, 130.90, 131.92, 132.40, 135.30, 136.18, 137.14, 138.99, 139.46, 141.54, 146.80, 147.05, 167.55, 168.71, 169.50, 171.37, 171.93.

### Preparation of 3-(2-(2-(2-((2-(2,6-Dioxopiperidin-3-yl)-1,3-dioxoisoindolin-4-yl)amino)ethoxy)ethoxy)ethoxy)-N-(3-((3-((trans)-4-hydroxycyclohexyl)-4-imino-5,6-diphenyl-3,4-dihydro-7H-pyrrolo[2,3-d]pyrimidin-7-yl)methyl)benzyl)propanamide (2C-5)

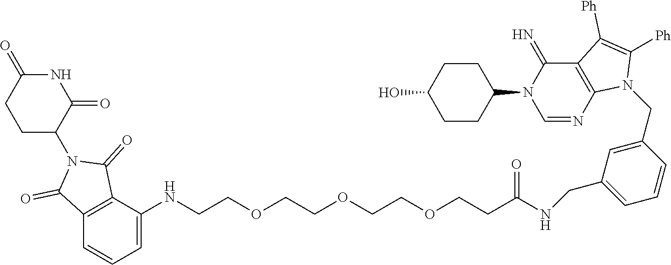

tert-Butyl (3-((3-((trans)-4-hydroxycyclohexyl)-4-imino-5,6-diphenyl-3,4-dihydro-7H-pyrrolo[2,3-d]pyrimidin-7-yl)methyl)benzyl)carbamate (14.8 mg, 1 Eq, 24.5 μmol) was treated with TFA/DCM (1/1, 1 mL), and stirred for 1 hour. The solvent was removed in vacuo, and N-ethyl-N-isopropylpropan-2-amine (12.7 mg, 4 Eq, 98.0 μmol) and 3-(2-(2-(2-((2-(2,6-dioxopiperidin-3-yl)-1,3-dioxoisoindolin-4-yl)amino)ethoxy)ethoxy)ethoxy)propanoic acid (11.7 mg, 1 Eq, 24.5 μmol) were added. Then, HATU (9.32 mg, 1 Eq, 24.5 μmol) was added to the above stirred reaction solution, and stirred at room temperature for 2 hours. DMF was removed under a stream of N₂, and the reaction mixture was purified by flash chromatography on silica gel (methanol/DCM: 0 to 355%) to give the desired product (8.8 mg, 9.1 μmol, 37% yield). ¹H NMR (400 MHz, CDCl₃) δ 1.40-1.56 (m, 2H), 1.79-1.89 (m, 2H), 1.99-2.19 (m, 5H), 2.31 (d, J=10.7 Hz, 2H), 2.48 (t, J=5.7 Hz, 2H), 2.71-2.88 (m, 3H), 3.42 (t, J=5.4 Hz, 2H), 3.57-3.60 (m, 7H), 3.65 (t, J=5.5 Hz, 2H), 3.73 (t, J=6.1 Hz, 2H), 4.33 (d, J=6.0 Hz, 2H), 4.85-4.96 (m, 1H), 5.36 (s, 2H), 5.54 (s, 1H), 6.46 (d, J=4.6 Hz, 1H), 6.72 (d, J=6.5 Hz, 1H), 6.80 (s, 1H), 6.85-6.91 (m, 2H), 6.99-7.20 (m, 7H), 7.27-7.38 (m, 6H), 7.47 (d, J=7.3 Hz, 1H), 8.12 (d, J=8.3 Hz, 1H), 8.28 (s, 1H), 8.76 (s, 1H). ¹³C NMR (101 MHz, CDCl₃) δ 22.82, 31.36, 31.41, 33.80, 36.88, 42.33, 42.92, 46.67, 48.88, 57.73, 67.12, 69.36, 69.52, 70.16, 70.37, 70.40, 70.78, 100.43, 110.33, 111.70, 116.71, 117.13, 119.25, 125.86, 126.44, 126.90, 127.83, 128.56, 128.63, 128.95, 129.39, 130.27, 130.83, 130.92, 132.51, 136.06, 136.43, 138.41, 139.36, 140.85, 145.41, 146.72, 148.30, 151.54, 167.54, 168.53, 169.27, 171.21, 171.58.

### Example 4. Synthesis of Representative Metarrestin Analogues 3C-1 to 3C-5

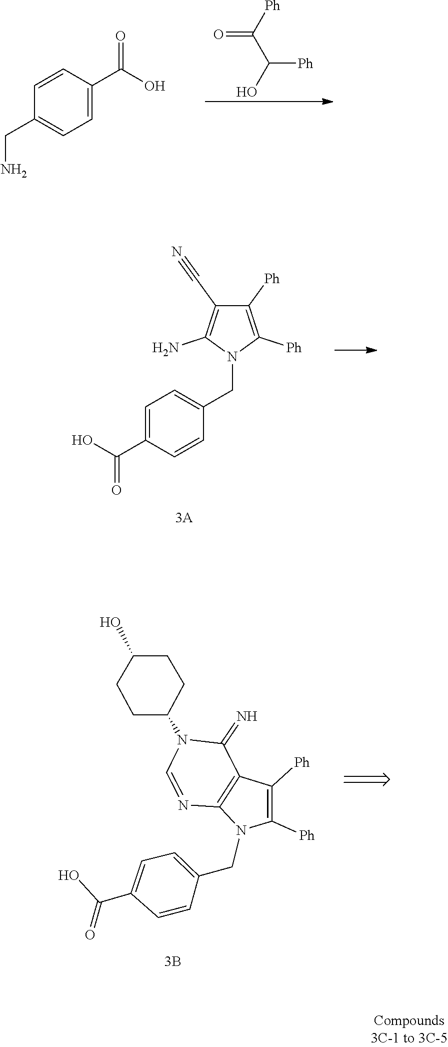 [SMILES: NCc1ccc(C(=O)O)cc1 ; O=C(c1ccccc1)C(O)c1ccccc1 ; N#Cc1c(-c2ccccc2)c(-c2ccccc2)n(Cc2ccc(C(=O)O)cc2)c1N ; N=c1c2c(-c3ccccc3)c(-c3ccccc3)n(Cc3ccc(C(=O)O)cc3)c2ncn1[C@H]1CC[C@@H](O)CC1]

### Preparation of 4-((2-Amino-3-cyano-4,5-diphenyl-1H-pyrrol-1-yl)methyl)benzoic acid (3A)

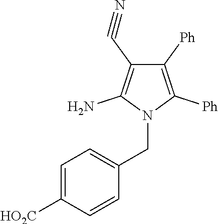 [SMILES: N#Cc1c(-c2ccccc2)c(-c2ccccc2)n(Cc2ccc(C(=O)O)cc2)c1N]

A suspension of the 4-(aminomethyl)benzoic acid (765.24 mg, 1 Eq, 5.0624 mmol) and acyloin 2-hydroxy-1,2-diphenylethan-1-one (1.0745 g, 1 Eq, 5.0624 mmol) in benzene (8 mL) was heated under microwave irradiation (120° C.) for 0.5 hour. Then, benzene was removed in vacuo, toluene (50 mL), malononitrile (334.4 mg, 1 Eq, 5.0624 mmol) and potassium tert-butoxide (568.05 mg, 1 Eq, 5.0624 mmol) were added, and the reaction was refluxed for 24 hours. The solvent was removed in vacuo, and purified by C18 chromatography (methanol/basic water: 10% to 100%) to afford the 2-amino-1H-pyrrole-3-carbonitrile derivative (0.1936 g, 492.1 μmol, 9.7% yield). ¹H NMR (400 MHz, CD₃OD) δ 7.86 (d, J=8.2 Hz, 2H), 7.27-7.11 (m, 8H), 7.06 (dd, J=7.9, 1.5 Hz, 2H), 6.95 (d, J=8.2 Hz, 2H), 5.01 (s, 2H). ¹³C NMR (101 MHz, CD₃OD) δ 174.07, 150.06, 141.19, 135.15, 132.74, 132.58, 130.78, 130.07, 129.61, 129.13, 127.39, 126.80, 126.22, 122.51, 119.42, 111.52, 93.30, 73.41, 47.36.

### Preparation of 4-((3-((trans)-4-Hydroxycyclohexyl)-4-imino-5,6-diphenyl-3,4-dihydro-7H-pyrrolo[2,3-d]pyrimidin-7-yl)methyl)benzoic acid (3B)

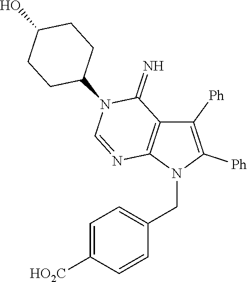

4-((2-Amino-3-cyano-4,5-diphenyl-1H-pyrrol-1-yl)methyl)benzoic acid (0.3592 g, 1 Eq, 912.9 μmol) in triethoxymethane (2.706 g, 20 Eq, 18.26 mmol) was refluxed for 4 h, and concentrated to dryness. The resulting imido ester was treated with (trans)-4-aminocyclohexan-1-ol (315.5 mg, 3 Eq, 2.739 mmol), and potassium tert-butoxide (204.9 mg, 2 Eq, 1.826 mmol) in MeOH (20 mL) was heated at 50° C. for 24 h, and then cooled to room temperature and filtered. The solvent was removed in vacuo, and purified by C18 chromatography (methanol/basic water: 10% to 100%) to afford the pyrrolopyrimidine product (153.9 mg, 296.7 μmol, 32.5% yield). ¹H NMR (400 MHz, CD₃OD) δ 8.20 (s, 1H), 7.87 (d, J=8.1 Hz, 2H), 7.38-7.10 (m, 8H), 7.08 (t, J=6.4 Hz, 2H), 6.97 (d, J=8.1 Hz, 2H), 5.11 (s, 2H), 3.72 (br s, 1H), 3.62-3.45 (m, 1H), 1.93 (dd, J=27.6, 10.2 Hz, 4H), 1.37-1.16 (m, 4H). ¹³C NMR (101 MHz, CD₃OD) δ 170.06, 153.34, 152.20, 144.92, 134.94, 132.69, 132.45, 130.91, 130.28, 129.64, 129.27, 129.17, 128.38, 127.52, 123.70, 120.44, 111.48, 76.80, 70.46, 50.31, 47.47, 34.64, 30.92.

### Preparation of N-(5-((2-(2,6-Dioxopiperidin-3-yl)-1,3-dioxoisoindolin-4-yl)amino)pentyl)-4-((3-((trans)-4-hydroxycyclohexyl)-4-imino-5,6-diphenyl-3,4-dihydro-7H-pyrrolo[2,3-d]pyrimidin-7-yl)methyl)benzamide (3C-1)

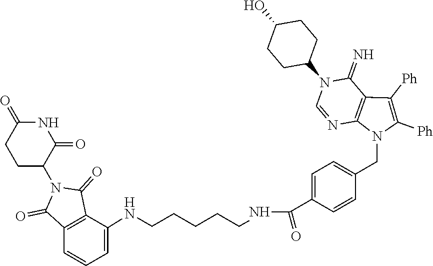

4-((3-((trans)-4-Hydroxycyclohexyl)-4-imino-5,6-diphenyl-3,4-dihydro-7H-pyrrolo[2,3-d]pyrimidin-7-yl)methyl)benzoic acid (15.2 mg, 1 Eq, 29.4 μmol) in DMF (2 mL) was treated with N-ethyl-N-isopropylpropan-2-amine (15.2 mg, 4 Eq, 118 μmol) and 4-((5-(chloro-15-azaneyl)pentyl)amino)-2-(2,6-dioxopiperidin-3-yl)isoindoline-1,3-dione (11.6 mg, 1 Eq, 29.4 μmol). Then, HATU (11.2 mg, 1 Eq, 29.4 μmol) was added to the above stirred reaction solution, and stirred at room temperature for 2 hours. DMF was removed under a stream of N₂, and the reaction mixture was purified by flash chromatography on silica gel (methanol/DCM: 0 to 25%) to give the desired product (10.3 mg, 12.0 μmol, 40.8% yield). ¹H NMR (400 MHz, CD₃OD) δ 8.66 (s, 1H), 7.61 (d, J=8.2 Hz, 2H), 7.47 (dd, J=8.4, 7.3 Hz, 1H), 7.42-7.21 (m, 8H), 7.14 (d, J=7.2 Hz, 2H), 7.03-6.89 (m, 4H), 5.53 (s, 2H), 5.01 (dd, J=12.6, 5.5 Hz, 1H), 4.58-4.41 (m, 1H), 3.82-3.70 (m, 1H), 3.37-3.30 (m, 4H), 2.64-2.78 (m, 2H), 2.04-2.17 (m, 8H), 1.58-1.72 (m, 6H), 1.52-1.42 (m, 2H), 1.41-1.35 (m, 2H). ¹³C NMR (101 MHz, CD₃OD) δ 174.65, 171.65, 170.80, 169.51, 169.33, 153.03, 148.32, 147.38, 143.49, 141.51, 139.86, 137.29, 135.29, 133.90, 133.15, 132.22, 131.86, 130.54, 130.32, 130.21, 129.75, 129.66, 128.65, 128.27, 118.36, 118.09, 111.78, 110.98, 101.87, 69.60, 58.99, 55.91, 50.24, 47.30, 43.29, 40.82, 35.05, 32.31, 31.20, 30.17, 30.08, 25.30, 23.88.

### Preparation of N-(2-(2-((2-(2,6-Dioxopiperidin-3-yl)-1,3-dioxoisoindolin-4-yl)amino)ethoxy)ethyl)-4-((3-((trans)-4-hydroxycyclohexyl)-4-imino-5,6-diphenyl-3,4-dihydro-7H-pyrrolo[2,3-d]pyrimidin-7-yl)methyl)benzamide (3C-2)

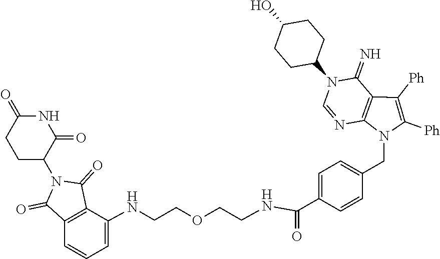

4-((3-((trans)-4-Hydroxycyclohexyl)-4-imino-5,6-diphenyl-3,4-dihydro-7H-pyrrolo[2,3-d]pyrimidin-7-yl)methyl)benzoic acid (13.5 mg, 1 Eq, 26.0 μmol) in DMF (2 mL) was treated with N-ethyl-N-isopropylpropan-2-amine (13.4 mg, 4 Eq, 104 μmol) and 4-((2-(2-(chloro-l5-azaneyl)ethoxy)ethyl)amino)-2-(2,6-dioxopiperidin-3-yl)isoindoline-1,3-dione (10.3 mg, 1 Eq, 26.0 μmol). Then, HATU (9.87 mg, 1 Eq, 26.0 μmol) was added to the above stirred reaction solution, and stirred at room temperature for 2 hours. DMF was removed under a stream of N₂, and the reaction mixture was purified by flash chromatography on silica gel (methanol/DCM. 0 to 35%) to give the desired product (7.9 mg, 9.2 μmol, 35% yield). ¹H NMR (400 MHz, CDCl₃) δ 1.33-1.59 (m, 4H), 1.78-2.27 (m, 9H), 2.56-2.79 (m, 2H), 3.40-3.45 (m, 2H), 3.54-3.85 (m, 6H), 4.85 (dd, J=12.4, 5.4 Hz, 1H), 5.28-5.53 (m, 3H), 6.54 (t, J=5.4 Hz, 1H), 6.88-6.92 (m, 3H), 7.00-7.01 (m, 3H), 7.10-7.37 (m, 9H), 7.41 (dd, J=8.5, 7.1 Hz, 1H), 7.69 (d, J=8.4 Hz, 2H), 8.38 (s, 1H), 9.44 (s, 1H). ¹³C NMR (101 MHz, CDCl₃) δ 22.62, 31.01, 31.06, 31.43, 33.69, 39.72, 41.89, 42.22, 46.40, 48.89, 50.53, 53.58, 69.08, 69.15, 69.78, 100.62, 110.23, 111.73, 116.98, 117.12, 126.90, 127.64, 128.40, 128.51, 128.62, 129.29, 130.26, 130.70, 131.14, 132.26, 133.87, 136.11, 139.47, 141.91, 145.05, 146.82, 151.57, 167.00, 167.46, 168.79, 169.48, 171.66.

### Preparation of N-(2-(2-(2-((2-(2,6-Dioxopiperidin-3-yl)-1,3-dioxoisoindolin-4-yl)amino)ethoxy)ethoxy)ethyl)-4-((3-((trans)-4-hydroxycyclohexyl)-4-imino-5,6-diphenyl-3,4-dihydro-7H-pyrrolo[2,3-d]pyrimidin-7-yl)methyl)benzamide (3C-3)

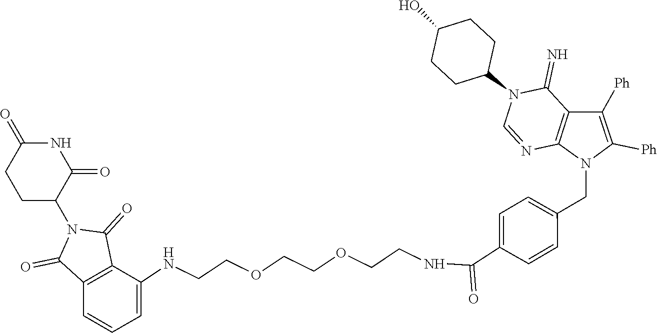

4-((3-((trans)-4-Hydroxycyclohexyl)-4-imino-5,6-diphenyl-3,4-dihydro-7H-pyrrolo[2,3-d]pyrimidin-7-yl)methyl)benzoic acid (13.9 mg, 1 Eq, 26.8 μmol) in DMF (2 mL) was treated with N-ethyl-N-isopropylpropan-2-amine (13.8 mg, 4 Eq, 107 μmol) and 4-((2-(2-(2-(chloro-l5-azaneyl)ethoxy)ethoxy)ethyl)amino)-2-(2,6-dioxopiperidin-3-yl)isoindoline-1,3-dione (11.8 mg, 1 Eq, 26.8 μmol). Then, HATU (10.2 mg, 1 Eq, 26.8 μmol) was added to the above stirred reaction solution, and stirred at room temperature for 2 hours. DMF was removed under a stream of N₂, and the reaction mixture was purified by flash chromatography on silica gel (methanol/DCM. 0 to 25%) to give the desired product (14.9 mg, 16.5 μmol, 61.5% yield). ¹H NMR (400 MHz, CDCl₃) δ 1.38-1.57 (m, 2H), 1.86 (d, J=11.4 Hz, 4H), 1.95-2.29 (m, 6H), 2.56-2.78 (m, 2H), 3.35-3.50 (m, 3H), 3.56-3.77 (m, 8H), 4.73 (dd, J=12.0, 5.4 Hz, 1H), 5.36 (s, 2H), 5.55 (t, J=12.4 Hz, 1H), 6.52 (t, J=5.3 Hz, 1H), 6.83-6.90 (m, 3H), 6.99-7.05 (m, 3H), 7.09-7.38 (m, 8H), 7.39-7.48 (m, 1H), 7.70 (d, J=8.2 Hz, 2H), 8.32 (s, 1H), 9.00 (s, 1H). ¹³C NMR (101 MHz, CDCl₃) δ 22.6, 31.2, 31.3, 33.6, 39.8, 42.1, 46.4, 48.8, 50.6, 53.6, 57.7, 69.0, 69.2, 69.8, 70.0, 70.5, 100.4, 110.2, 111.6, 116.7, 117.1, 126.9, 127.6, 128.2, 128.7, 128.7, 129.4, 130.2, 130.6, 130.9, 132.4, 134.2, 136.1, 139.3, 141.4, 145.4, 146.6, 151.3, 166.8, 167.5, 168.6, 169.4, 171.3.

### Preparation of N-(2-((2-(2,6-Dioxopiperidin-3-yl)-1,3-dioxoisoindolin-4-yl)amino)ethyl)-4-((3-((trans)-4-hydroxycyclohexyl)-4-imino-5,6-diphenyl-3,4-dihydro-7H-pyrrolo[2,3-d]pyrimidin-7-yl)methyl)benzamide (3C-4)

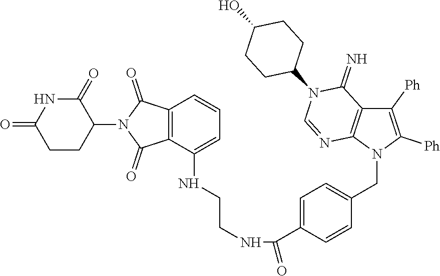

4-((3-((trans)-4-Hydroxycyclohexyl)-4-imino-5,6-diphenyl-3,4-dihydro-7H-pyrrolo[2,3-d]pyrimidin-7-yl)methyl)benzoic acid (16.0 mg, 1 Eq, 30.9 μmol) in DMF (2 mL) was treated with N-ethyl-N-isopropylpropan-2-amine (16.0 mg, 4 Eq, 124 μmol) and 4-((2-(chloro-l5-azaneyl)ethyl)amino)-2-(2,6-dioxopiperidin-3-yl)isoindoline-1,3-dione (10.9 mg, 1 Eq, 30.9 μmol). Then, HATU (11.7 mg, 1 Eq, 30.9 μmol) was added to the above stirred reaction solution, and stirred at room temperature for 2 hours. DMF was removed under a stream of N₂, and the reaction mixture was purified by flash chromatography on silica gel (methanol/DCM: 0 to 25%) to give the desired product (14.7 mg, 18.0 μmol, 58.2% yield). ¹H NMR (400 MHz, CD₃OD) δ 1.33-1.40 (m, 2H), 1.53-1.71 (m, 2H), 1.96-2.28 (m, 6H), 2.60-2.73 (m, 2H), 2.77-2.87 (m, 1H), 3.56 (s, 4H), 3.71-3.81 (m, 1H), 4.45-4.53 (m, 1H), 5.02 (dd, J=12.5, 5.5 Hz, 1H), 5.52 (s, 2H), 6.90-6.99 (m, 3H), 7.12-7.14 (m, 3H), 7.24-7.50 (m, 8H), 7.61 (d, J=8.3 Hz, 2H), 8.67 (s, 1H). ¹³C NMR (101 MHz, CD₃OD) δ 23.8, 31.1, 32.2, 35.0, 40.5, 42.6, 47.2, 50.1, 55.8, 59.0, 69.5, 101.7, 111.4, 112.0, 118.2, 118.3, 128.2, 128.7, 129.6, 129.7, 130.1, 130.3, 130.5, 131.8, 132.1, 133.0, 133.9, 134.9, 137.1, 139.8, 141.6, 143.4, 147.3, 148.2, 152.9, 169.2, 170.0, 170.5, 171.6, 174.6.

### Preparation of (2S,4S)-1-((S)-2-Cyclohexyl-2-((S)-2-(methylamino)propanamido)acetyl)-4-(4-((3-((trans)-4-hydroxycyclohexyl)-4-imino-5,6-diphenyl-3,4-dihydro-7H-pyrrolo[2,3-d]pyrimidin-7-yl)methyl)benzamido)-N-((R)-1,2,3,4-tetrahydronaphthalen-1-yl)pyrrolidine-2-carboxamide (3C-5)

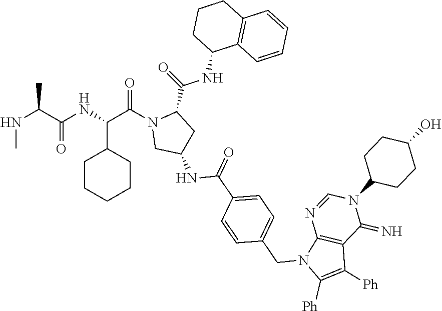

4-((3-((trans)-4-Hydroxy cyclohexyl)-4-imino-5,6-diphenyl-3,4-dihydro-7H-pyrrolo[2,3-d]pyrimidin-7-yl)methyl)benzoic acid (9.78 mg, 1 Eq, 18.9 μmol) in DMF (2 mL) was treated with N-ethyl-N-isopropylpropan-2-amine (9.75 mg, 4 Eq, 75.5 μmol) and tert-butyl ((S)-1-(((S)-2-((2S,4S)-4-(chloro-15-azaneyl)-2-(((R)-1,2,3,4-tetrahydronaphthalen-1-yl)carbamoyl)pyrrolidin-1-yl)-1-cyclohexyl-2-oxoethyl)amino)-1-oxopropan-2-yl)(methyl)carbamate (11.7 mg, 1 Eq, 18.9 μmol). Then, HATU (7.17 mg, 1 Eq, 18.9 μmol) was added to the above stirred reaction solution, and stirred at room temperature for 2 hours. DMF was removed under a stream of N₂, and the reaction mixture was treated with TFA/DCM (1/1, 2 mL) for 1 hour at room temperature. The solvent was removed in vacuo, and purified by flash chromatography on silica gel (methanol/DCM: 0 to 25%) to give the desired product (6.1 mg, 6.2 μmol, 33% yield). ¹H NMR (400 MHz, CD₃OD) δ 0.96-1.20 (m, 6H), 1.25-1.41 (m, 2H), 1.47 (d, J=6.9 Hz, 3H), 1.51-2.25 (m, 14H), 2.49-2.61 (m, 1H), 2.65 (s, 3H), 2.73-2.86 (m, 2H), 3.35 (s, 3H), 3.67-3.86 (m, 2H), 3.89 (q, J=7.0 Hz, 1H), 4.05 (dd, J=10.7, 4.8 Hz, 1H), 4.40-4.52 (m, 3H), 4.71 (s, 1H), 5.08 (t, J=6.6 Hz, 1H), 5.56 (s, 2H), 6.97-7.03 (m, 2H), 7.03-7.17 (m, 4H), 7.23-7.43 (m, 8H), 7.70-7.79 (m, 2H), 8.61 (d, J=9.1 Hz, 1H), 8.69 (s, 1H), 9.21 (d, J=8.0 Hz, 1H). ¹³C NMR (101 MHz, CD₃OD) δ 16.23, 21.64, 26.98, 27.13, 27.18, 29.97, 30.20, 30.27, 31.07, 31.17, 31.81, 34.88, 35.26, 41.42, 47.21, 49.85, 50.92, 55.45, 57.48, 58.18, 59.19, 60.83, 69.48, 101.63, 118.25, 127.03, 128.15, 128.40, 128.63, 129.65, 129.67, 129.77, 129.91, 130.03, 130.26, 130.51, 131.75, 132.10, 132.91, 134.41, 137.52, 138.51, 140.07, 141.95, 143.29, 147.54, 152.67, 168.36, 170.15, 172.27, 173.85.

### Preparation of (trans)-4-(7-benzyl-4-imino-5,6-diphenyl-4,7-dihydro-3H-pyrrolo[2,3-d]pyrimidin-3-yl)cyclohexyl 4-((2-(2,6-dioxopiperidin-3-yl)-1,3-dioxoisoindolin-4-yl)amino)butanoate

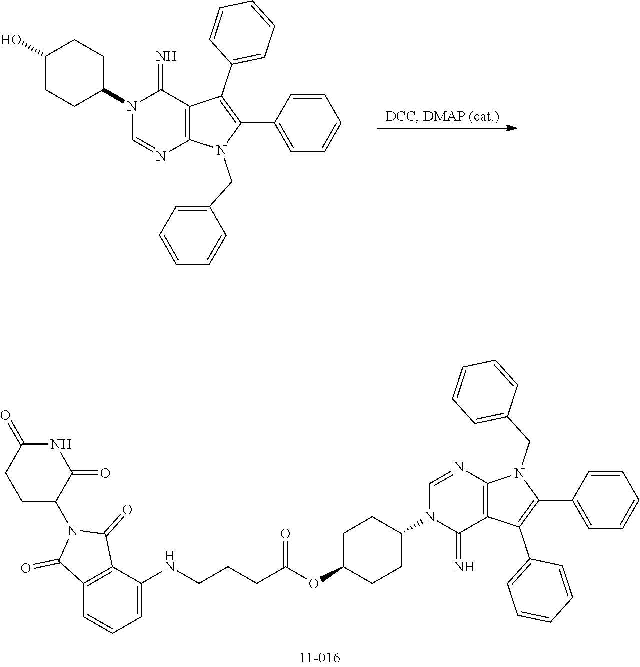 [SMILES: N=c1c2c(-c3ccccc3)c(-c3ccccc3)n(Cc3ccccc3)c2ncn1[C@H]1CC[C@H](O)CC1 ; N=c1c2c(-c3ccccc3)c(-c3ccccc3)n(Cc3ccccc3)c2ncn1[C@H]1CC[C@H](OC(=O)CCCNc2cccc3c2C(=O)N(C2CCC(=O)NC2=O)C3=O)CC1]

Dicyclohexylcarbodiimide (7.52 mg, 1.1 Eq, 36.4 mol) was added to a suspension of 4-((2-(2,6-dioxopiperidin-3-yl)-1,3-dioxoisoindolin-4-yl)amino)butanoic acid (11.9 mg, 1 Eq, 33.1 mol), (trans)-4-(7-(3-bromobenzyl)-4-imino-5,6-diphenyl-4,7-dihydro-3H-pyrrolo[2,3-d]pyrimidin-3-yl)cyclohexan-1-ol (18.3 mg, 1 Eq, 33.1 mol) and N,N-dimethylpyridin-4-amine (809 g, 0.2 Eq, 6.62 mol) in CH₂Cl₂ (3 mL) at room temperature. The mixture was stirred continuously for 12 hours, and the solvents removed in vacuo. The mixture was purified by reverse phase (CH₃CN/water) followed by normal phase (methanol/DCM) column chromatography to give (trans)-4-(7-benzyl-4-imino-5,6-diphenyl-4,7-dihydro-3H-pyrrolo[2,3-d]pyrimidin-3-yl)cyclohexyl 4-((2-(2,6-dioxopiperidin-3-yl)-1,3-dioxoisoindolin-4-yl)amino)butanoate as yellowish solid (4.9 mg, 6.0 mol, 18% yield). ¹H NMR (500 MHz, CDCl₃) δ 1.80-1.92 (m, 3H), 1.96-2.06 (m, 3H), 2.10-2.17 (m, 3H), 2.27 (s, 2H), 2.44 (t, J=7.0 Hz, 2H), 2.70-2.81 (m, 1H), 2.82-2.93 (m, 1H), 3.38 (qd, J=6.9, 2.3 Hz, 2H), 4.75 (t, J=15.5 Hz, 1H), 4.94 (dd, J=12.3, 5.3 Hz, 1H), 5.39 (s, 2H), 5.59 (s, 2H), 6.23 (s, 1H), 6.33 (t, J=5.9 Hz, 1H), 6.88-6.99 (m, 2H), 7.04-7.09 (m, 1H), 7.11 (d, J=7.1 Hz, 1H), 7.18-7.39 (m, 10H), 7.49-7.59 (m, 2H), 8.19 (s, 4H). ¹³C NMR (126 MHz, CDCl₃) δ 22.7, 24.4, 29.7, 30.7, 31.4, 31.9, 40.9, 42.0, 46.7, 48.9, 71.8, 76.7, 77.0, 77.3, 110.1, 111.6, 116.7, 127.0, 127.9, 128.5, 128.7, 129.2, 130.3, 130.8, 132.5, 136.3, 146.7, 167.6, 168.4, 169.3, 171.0, 172.7.

### Preparation of (trans)-4-(7-benzyl-4-imino-5,6-diphenyl-4,7-dihydro-3H-pyrrolo[2,3-d]pyrimidin-3-yl)cyclohexyl 3-(2-(2-(2-((2-(2,6-dioxopiperidin-3-yl)-1,3-dioxoisoindolin-4-yl)amino)ethoxy)ethoxy)ethoxy)propanoate

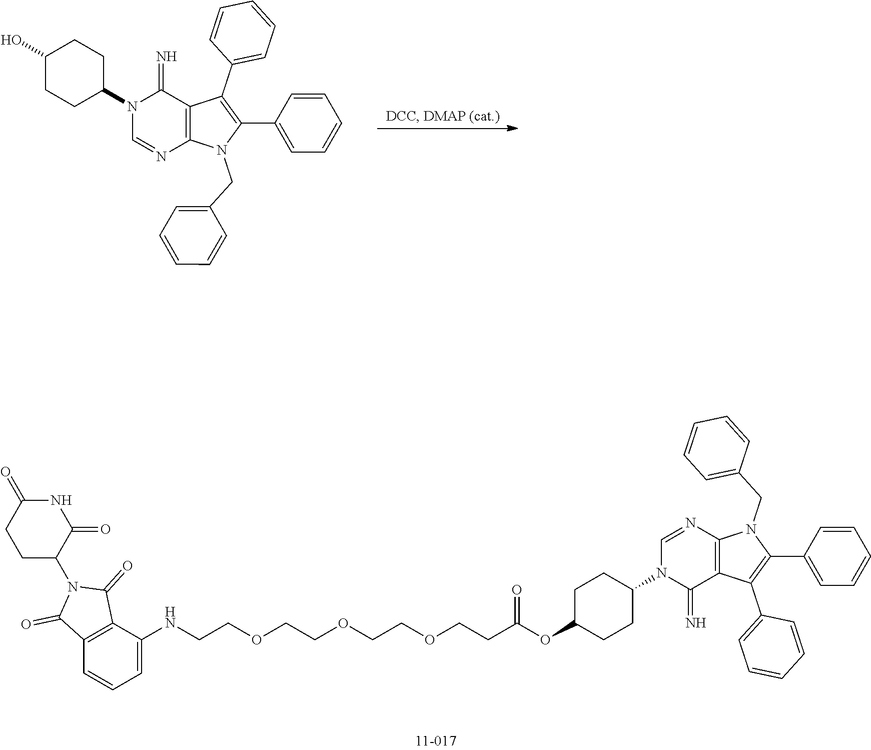

Dicyclohexylcarbodiimide (5.75 mg, 1.1 Eq, 27.9 mol) was added to a suspension of 3-(2-(2-(2-((2-(2,6-dioxopiperidin-3-yl)-1,3-dioxoisoindolin-4-yl)amino)ethoxy)ethoxy)ethoxy)propanoic acid (12.1 mg, 1 Eq, 25.3 mol), (trans)-4-(7-benzyl-4-imino-5,6-diphenyl-4,7-dihydro-3H-pyrrolo[2,3-d]pyrimidin-3-yl)cyclohexan-1-ol (12.0 mg, 1 Eq, 25.3 mol) and N,N-dimethylpyridin-4-amine (619 g, 0.2 Eq, 5.07 mol) in CH₂Cl₂ (3 mL) at room temperature. The mixture was stirred continuously for 12 hours, and the solvents removed in vacuo. The mixture was purified by reverse phase (CH₃CN/water) followed by normal phase (methanol/DCM) column chromotography to give (trans)-4-(7-benzyl-4-imino-5,6-diphenyl-4,7-dihydro-3H-pyrrolo[2,3-d]pyrimidin-3-yl)cyclohexyl 3-(2-(2-(2-((2-(2,6-dioxopiperidin-3-yl)-1,3-dioxoisoindolin-4-yl)amino)ethoxy)ethoxy)ethoxy)propanoate (7.85 mg, 8.40 mol, 33.2% yield) as yellowish solid.

¹H NMR (500 MHz, CDCl₃) δ 1.78-1.86 (m, 2H), 1.98-2.18 (m, 6H), 2.26 (d, J=9.9 Hz, 2H), 2.56 (t, J=6.4 Hz, 2H), 2.70-2.79 (m, 2H), 2.83-2.89 (m, 1H), 3.46 (q, J=5.5 Hz, 2H), 3.58-3.68 (m, 8H), 3.70-3.75 (m, 4H), 4.76-4.83 (m, 1H), 4.91 (dd, J=12.2, 5.4 Hz, 1H), 5.36 (s, 2H), 5.50 (s, 1H), 6.49 (t, J=5.6 Hz, 1H), 6.86-6.95 (m, 3H), 7.01-7.10 (m, 3H), 7.15-7.36 (m, 11H), 7.47 (dd, J=8.4, 7.3 Hz, 1H), 8.14 (s, 2H). ¹³C NMR (126 MHz, CDCl₃) δ 22.7, 29.9, 30.8, 31.4, 35.3, 42.4, 46.6, 48.8, 66.5, 69.4, 70.4, 70.5, 70.6, 70.7, 71.5, 76.7, 77.0, 77.3, 110.2, 111.5, 116.8, 117.3, 127.0, 127.8, 128.5, 128.6, 129.2, 130.3, 130.8, 132.4, 136.0, 143.5, 146.8, 167.6, 168.4, 169.2, 171.2, 171.3.

### REFERENCES

1. https://www.cancer.org/latest-news/facts-and-figures-2019.html.
2. Gundem, G., et al., The evolutionary history of lethal metastatic prostate cancer. Nature, 2015. 520 (7547): p. 353-7.
3. Weber, G. F., Why does cancer therapy lack effective anti-metastasis drugs?Cancer Lett, 2013. 328 (2): p. 207-11.
4. Liao, B. C., C. C. Lin, and J. C. Yang, Second and third-generation epidermal growth factor receptor tyrosine kinase inhibitors in advanced nonsmall cell lung cancer. Curr Opin Oncol, 2015. 27(2): p. 94-101.
5. Frankowski, K. J., et al., Metarrestin, a perinucleolar compartment inhibitor, effectively suppresses metastasis. Sci Transl Med, 2018. 10(441).
6. Ueki, N., et al., Synthesis and Preclinical Evaluation of a Highly Improved Anticancer Prodrug Activated by Histone Deacetylases and Cathepsin L. Theranostics, 2016.6(6): 15 p. 808-16.
7. Ueki, N., et al., Selective cancer targeting with prodrugs activated by histone deacetylases and a tumour-associated protease. Nat Commun, 2013. 4: p. 2735.
8. Haberland, M., et al., Genetic dissection of histone deacetylase requirement in tumor cells. Proc Natl Acad Sci USA, 2009. 106(19): p. 7751-5.
9. Denhardt, D. T., et al., Cysteine proteinase cathepsin L expression correlates closely with the metastatic potential of H-ras-transformed murine fibroblasts. Oncogene, 1987. 2(1): p. 55-9.
10. Uhlen, M., et al., Proteomics. Tissue-based map of the human proteome. Science, 2015. 347(6220): p. 1260419.
11. Sasikumar, A. N., W. B. Perez, and T. G. Kinzy, The many roles of the eukaryotic elongation factor 1 complex. Wiley Interdiscip Rev RNA, 2012. 3(4): p. 543-55.
12. For metastatic cancer, see http://mbcn.org/incidence-and-incidence-rates/.
13. For breast cancer, see https://cancerstatisticscenter.cancer.org/?ga=2.122887307.1413890692.1539447737-1771967173.1539447737 #!/cancer-site/Breast.
14. Ma, J. and A. Jemal, The rise and fall of cancer mortality in the USA: why does pancreatic cancer not follow the trend?Future Oncol, 2013. 9(7): p. 917-9.
15. Rudloff, U., et al., Randomized clinical trials in pancreatic adenocarcinoma. Surg Oncol Clin N Am, 2010. 19(1): p. 115-50.
16. Siegel, R., D. Naishadham, and A. Jemal, Cancer statistics, 2013. CA Cancer J Clin, 2013. 63(1): p. 11-30.
17. Kamath, R. V., et al., Perinucleolar compartment prevalence has an independent prognostic value for breast cancer. Cancer Research, 2005. 65(1): p. 246-53.
18. Slusarczyk, A. and S. Huang, The perinucleolar compartment (PNC): detection by immunohistochemistry. Methods Mol Biol, 2008. 463: p. 161-7.
19. Norton, J. T., et al., Perinucleolar compartment prevalence is a phenotypic pancancer marker of malignancy. Cancer, 2008. 113(4): p. 861-9.
20. Wang, C., et al., RNA polymerase III transcripts and the PTB protein are essential for the integrity of the perinucleolar compartment. Mol Biol Cell, 2003. 14(6): p. 2425-35.
21. Matera, A. G., et al., A perinucleolar compartment contains several RNA polymerase III transcripts as well as the polypyrimidine tract-binding protein, hnRNPI. J Cell Biol, 1995. 129(5): p. 1181-93.
22. Lee, B., et al., Association of RNase mitochondrial RNA processing enzyme with ribonuclease P in higher ordered structures in the nucleolus: a possible coordinate role in ribosome biogenesis. Proc Natl Acad Sci USA, 1996. 93(21): p. 11471-6.
23. Yap, K., et al., A short tandem repeat-enriched RNA assembles a nuclear compartment to control alternative splicing and promote cell survival. Mol Cell, 2018.
24. Huang, S., et al., The perinucleolar compartment and transcription. Journal of Cell Biology, 1998. 143(1): p. 35-47.
25. Norton, J. T., et al., The perinucleolar compartment is directly associated with DNA. Journal of Biological Chemistry, 2009. 284(7): p. 4090-101.
26. Slusarczyk, A., et al., Structure and function of the perinucleolar compartment in cancer cells. Cold Spring Harb Symp Quant Biol, 2011.
27. Norton, J. T., et al., Automated high-content screening for compounds that disassemble the perinucleolar compartment. J Biomol Screen, 2009. 14(9): p. 1045-53.
28. Frankowski, K., et al., Discovery and optimization of pyrrolopyrimidine derivatives as selective disruptors of the perinucleolar compartment, a marker of tumor progression toward metastasis. J. Med. Chem., 2022. in press.
29. Vilimas, T., et al., Pharmacokinetic evaluation of the PNC disassembler metarrestin in wild-type and Pdxl-Cre; LSL-Kras(G12D/+); Tp53(R172H/+) (KPC) mice, a genetically engineered model of pancreatic cancer. Cancer Chemother Pharmacol, 2018.
30. Toure, M. and C. M. Crews, Small-Molecule PROTACS: New Approaches to Protein Degradation. Angew Chem Int Ed Engl, 2016. 55(6): p. 1966-73.
31. Deshaies, R. J., Protein degradation: Prime time for PROTACs. Nat Chem Biol, 2015. 11(9): p. 634-5.
32. Békés, M., D. R. Langley, and C. M. Crews, PROTAC targeted protein degraders: the past is prologue. Nature Reviews Drug Discovery, 2022. 21(3): p. 181-200.
33. Zoppi, V., et al., Iterative Design and Optimization of Initially Inactive Proteolysis Targeting Chimeras (PROTACs) Identify VZ185 as a Potent, Fast, and Selective von Hippel-Lindau (VHL) Based Dual Degrader Probe of BRD9 and BRD7. Journal of Medicinal Chemistry, 2019. 62(2): p. 699-726.
34. Frankowski, K., et al., Discovery and development of small molecules that reduce PNC prevalence, in Probe Reports from the NIH Molecular Libraries Program. 2010: Bethesda (MD).
35. Zang, W., et al., miR-663 attenuates tumor growth and invasiveness by targeting eEF1A2 in pancreatic cancer. Mol Cancer, 2015. 14: p. 37.
36. Xu, C., D. M. Hu, and Q. Zhu, eEF1A2 promotes cell migration, invasion and metastasis in pancreatic cancer by upregulating MMP-9 expression through Akt activation. Clin Exp Metastasis, 2013. 30(7): p. 933-44.
37. Worst, T. S., et al., The EEF1A2 gene expression as risk predictor in localized prostate cancer. BMC Urology, 2017. 17(1): p. 86-86.
38. Van Goietsenoven, G., et al., Targeting of eEF1A with Amaryllidaceae isocarbostyrils as a strategy to combat melanomas. FASEB J, 2010. 24(11): p. 4575-84.
39. Tomlinson, V. A., et al., Expression of eEF1A2 is associated with clear cell histology in ovarian carcinomas: overexpression of the gene is not dependent on modifications at the EEF1A2 locus. Br J Cancer, 2007. 96(10): p. 1613-20.
40. Tomlinson, V. A., et al., Translation elongation factor eEF1A2 is a potential oncoprotein that is overexpressed in two-thirds of breast tumours. BMC Cancer, 2005. 5: p. 113.
41. Sun, Y., et al., Up-regulation of eEF1A2 promotes proliferation and inhibits apoptosis in prostate cancer. Biochemical and Biophysical Research Communications, 2014. 450(1): p. 1-6.
42. Scaggiante, B., et al., Dissecting the expression of EEF1A1/2 genes in human prostate cancer cells: the potential of EEF1A2 as a hallmarkfor prostate transformation and progression. Br J Cancer, 2012. 106(1): p. 166-73.
43. Lee, J. M., The role of protein elongation factor eEF1A2 in ovarian cancer. Reprod Biol Endocrinol, 2003. 1: p. 69.
44. Anand, N., et al., Protein elongation factor EEF1A2 is a putative oncogene in ovarian cancer. Nat Genet, 2002. 31(3): p. 301-5.
45. Abbas, W., A. Kumar, and G. Herbein, The eEF1A Proteins: At the Crossroads of Oncogenesis, Apoptosis, and Viral Infections. Front Oncol, 2015. 5: p. 75.
46. Jeganathan, S. and J. M. Lee, Binding of elongation factor eEF1A2 to phosphatidylinositol 4-kinase beta stimulates lipid kinase activity and phosphatidylinositol 4-phosphate generation. J Biol Chem, 2007. 282(1): p. 372-80.
47. Pinke, D. E. and J. M. Lee, The lipid kinase PI4KIIIbeta and the eEF1A2 oncogene co-operate to disrupt three-dimensional in vitro acinar morphogenesis. Exp Cell Res, 2011. 317(17): p. 2503-11.
48. Losada, A., et al., Binding of eEF1A2 to the RNA-dependent protein kinase PKR modulates its activity and promotes tumour cell survival. Br J Cancer, 2018. 119(11): p. 1410-1420.
49. Amiri, A., et al., eEF1A2 activates Akt and stimulates Akt-dependent actin remodeling, invasion and migration. Oncogene, 2007. 26(21): p. 3027-40.
50. Jia, L., et al., EEF1A2 interacts with HSP90ABI to promote lung adenocarcinoma metastasis via enhancing TGF-beta SMAD signalling. Br J Cancer, 2021. 124(7): p. 1301-1311.
51. Khacho, M., et al., eEF1A is a novel component of the mammalian nuclear protein export machinery. Mol Biol Cell, 2008. 19(12): p. 5296-308.
52. Li, L., et al., Expanded polyalanine tracts function as nuclear export signals and promote protein mislocalization via eEF1A1 factor. J Biol Chem, 2017. 292(14): p. 5784-5800.
53. https://www.proteinatlas.org.
54. Scott, D. E., et al., Systematic Investigation of the Permeability of Androgen Receptor PROTACs. ACS Medicinal Chemistry Letters, 2020. 11(8): p. 1539-1547.
55. Bricelj, A., et al., Influence of Linker Attachment Points on the Stability and Neosubstrate Degradation of Cereblon Ligands. ACS Medicinal Chemistry Letters, 2021. 12(11): p. 1733-1738.
56. Qiu, W. and G. H. Su, Development of orthotopic pancreatic tumor mouse models. Methods Mol Biol, 2013. 980: p. 215-23.
57. Samant, R. S., et al., Analysis of mechanisms underlying BRMS1 suppression of metastasis. Clin Exp Metastasis, 2000. 18(8): p. 683-93.
58. Suemizu, H., et al., Identification of a key molecular regulator of liver metastasis in human pancreatic carcinoma using a novel quantitative model of metastasis in NOD SCID/gammacnull (NOG) mice. Int J Oncol, 2007. 31(4): p. 741-51.
59. Lyons, S. K., Advances in imaging mouse tumour models in vivo. J Pathol, 2005. 205(2): p. 194-205.

1. A compound of Formula (I): 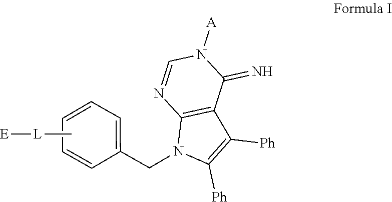
or an enantiomer, diastereomer, an enantiomeric mixture, a diastereomeric mixture or a pharmaceutically acceptable salt thereof; wherein:
A is selected from 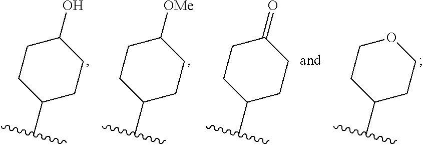
L is selected from 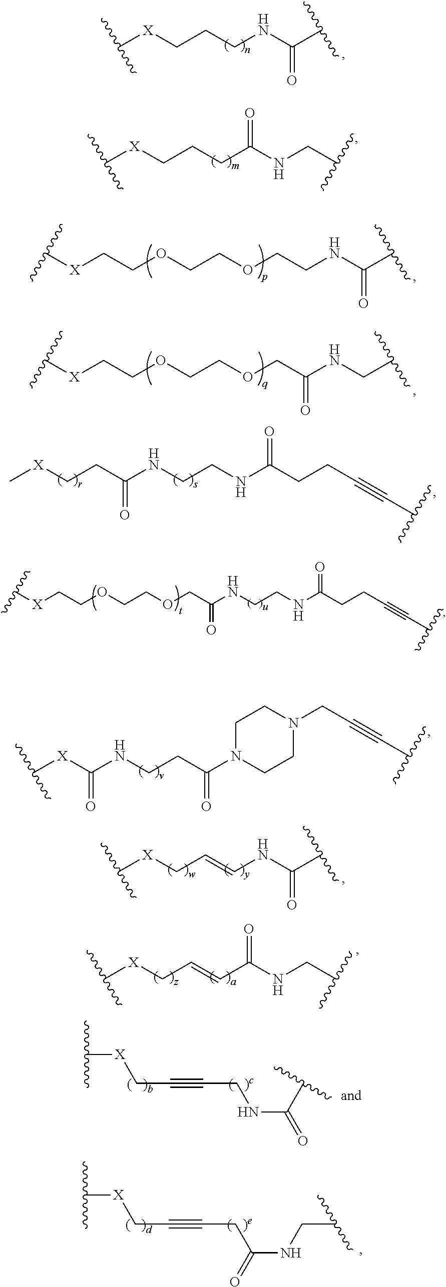
wherein a, b, c, d, e, n, m, p, q, r, s, t, u, v, w, y and z are an integer independently selected from 0, 1, 2, 3, 4, 5, 6, 7, 8, 9, and 10, and X is selected from —CH₂—, —NH—, and —O—; and
E is selected from

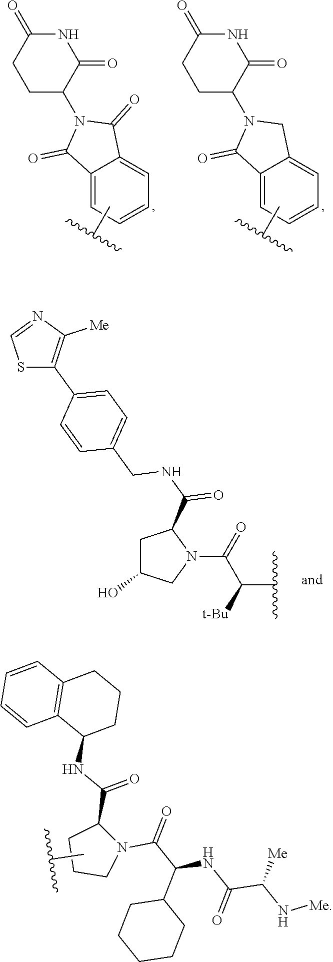

2. The compound of claim 1, wherein X is selected from —NH—, and —O—; and/or a, b, c, d, e, n, m, p, q, r, s, t, u, v, w, y and z are an integer independently selected from 1, 2, 3, 4, 5, 6, 7 and 8.

3. (canceled)

4. (canceled)

5. The compound of claim 1, wherein the compound is a compound of Formula II 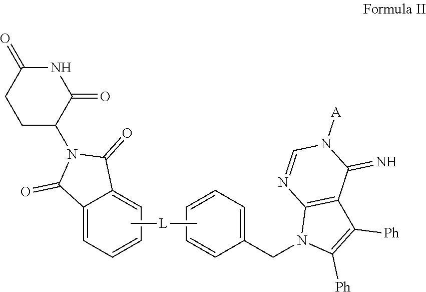
or an enantiomer, diastereomer, an enantiomeric mixture, a diastereomeric mixture or a pharmaceutically acceptable salt thereof; wherein:
A is selected from 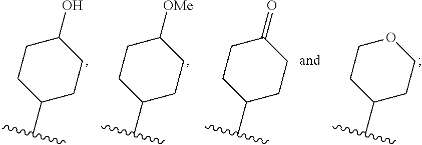
and
L is selected from 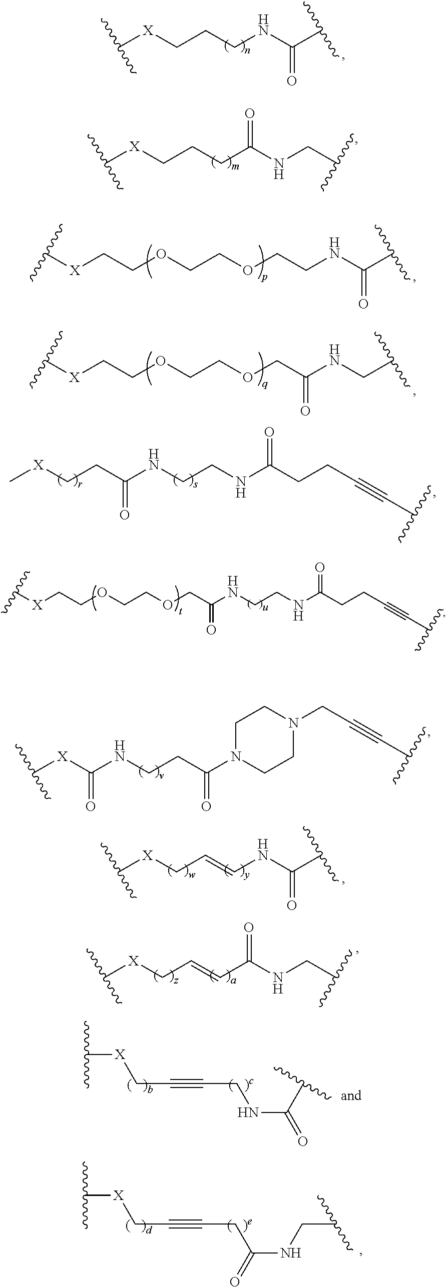
wherein a, b, c, d, e, n, m, p, q, r, s, t, u, y, w, y and z are an integer independently selected from 1, 2, 3, 4, 5, 6, 7, and 8, and X is selected from —NH—, and —O—.

6. The compound of claim 5, wherein A is 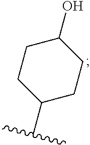 [SMILES: *C1CCC(O)CC1]
and/or
p, q, t, and v are an integer independently selected from 1, 2, 3, and 4.

7. (canceled)

8. (canceled)

9. The compound of claim 6, wherein the compound is a compound of Formula III 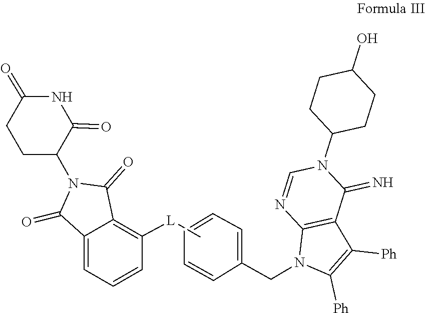
or an enantiomer, diastereomer, an enantiomeric mixture, a diastereomeric mixture or a pharmaceutically acceptable salt thereof; wherein L is selected from 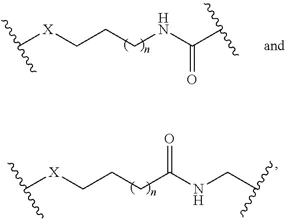
wherein n and m are an integer independently selected from 1, 2, 3, 4, 5, 6, 7, and 8; and X is selected from —NH—, and —O—.

10. The compound of claim 9, wherein n and m are an integer independently selected from 5, 6, 7, and 8.

11. The compound of claim 10, wherein X is —NH—.

12. (canceled)

13. (canceled)

14. (canceled)

15. (canceled)

16. The compound of claim 11, wherein the compound is

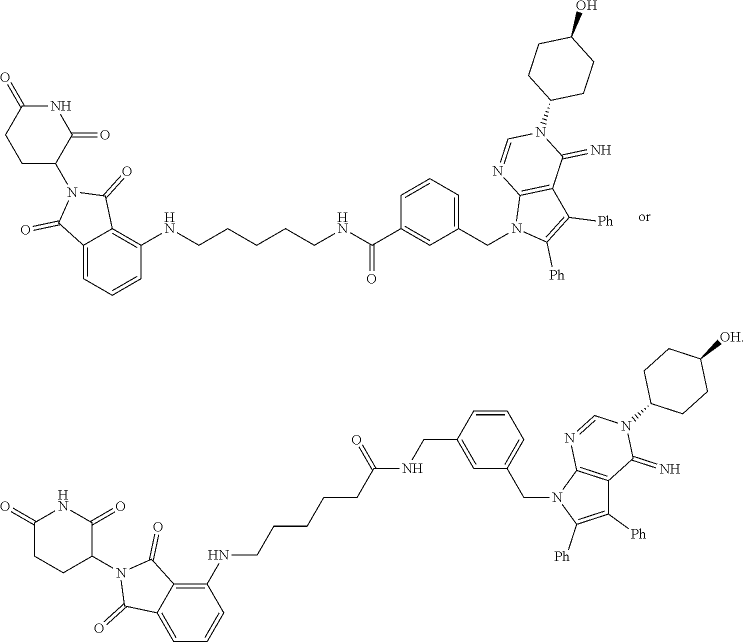 [SMILES: N=c1c2c(-c3ccccc3)c(-c3ccccc3)n(Cc3cccc(C(=O)NCCCCCNc4cccc5c4C(=O)N(C4CCC(=O)NC4=O)C5=O)c3)c2ncn1[C@H]1CC[C@H](O)CC1 ; N=c1c2c(-c3ccccc3)c(-c3ccccc3)n(Cc3cccc(CNC(=O)CCCCCNc4cccc5c4C(=O)N(C4CCC(=O)NC4=O)C5=O)c3)c2ncn1[C@H]1CC[C@H](O)CC1]

17. (canceled)

18. A compound of formula (IV): 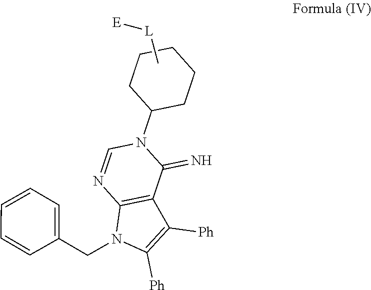
or an enantiomer, diastereomer, an enantiomeric mixture, a diastereomeric mixture or a pharmaceutically acceptable salt thereof; wherein:
L is selected from 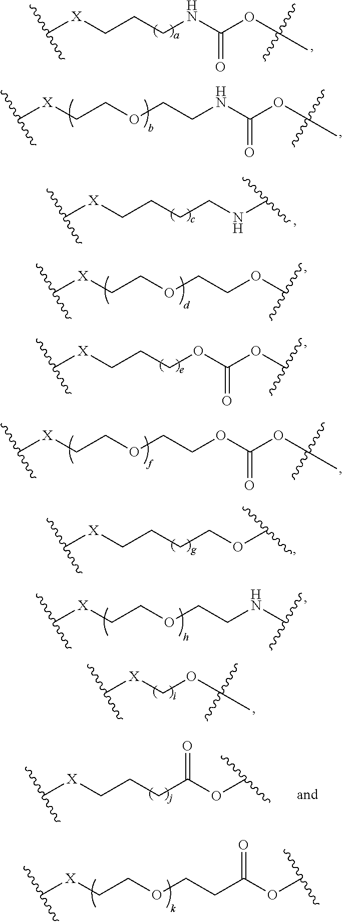
wherein a, b, c, d, e, f, g, h, i, j, and k are an integer independently selected from 0, 1, 2, 3, 4, 5, 6, 7, and 8, and X is selected from a bond —CH₂—, —NH—, and —O—, and
E is selected from

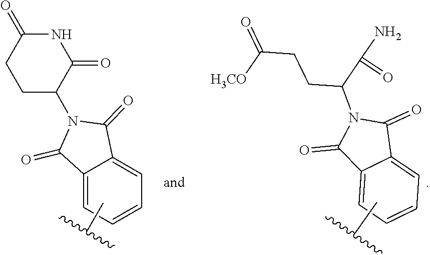

19. The compound of claim 18, wherein L is selected from 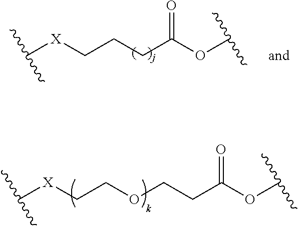
j and k, are an integer independently selected from 0, 1, 2, 3, 4, and 5.

20. The compound of claim 19, wherein X is —NH—.

21. The compound of claim 20, wherein j is 1 or 3; and k is 3.

22. The compound of claim 21, wherein E is

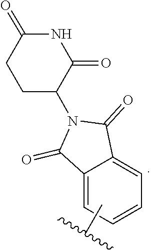

23. (canceled)

24. (canceled)

25. The compound of claim 21, wherein j is 1 and E is

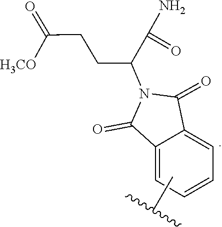

26. (canceled)

27. A method of reducing the eEF1A2 protein level in a cancer cell or tissue, the method comprising contacting the cancer cell or tissue with a compound of claim 1.

28. The method of claim 27, wherein the cancer tissue is selected from breast, prostate cancer, lung, kidney, thyroid, colon, pancreas and liver.

29. A method for treating a disease or condition that is treatable by inhibition of metarrestin targets, the method comprising administering to a subject in need thereof a therapeutically effective amount of a compound of claim 1.

30. The method of claim 29, wherein the metarrestin targets alter perinucleolar compartment (PNC) prevalence; and/or are selected from translation elongation factor eEF1A2 protein and/or RNA polymerase.

31. (canceled)

32. The method of claim 30, wherein the disease is cancer.

33. The method of claim 32, wherein the disease is a metastatic cancer selected from breast cancer, prostate cancer, lung cancer, kidney cancer, thyroid cancer, colon cancer, pancreatic cancer, bone cancer, and liver cancer.
